(19) United States
(12) Patent Application Publication
(10) Pub. No.: US 2025/0017946 A1
(43) Pub. Date: Jan. 16, 2025

(54) COMBINATION OF A BILE ACID AND A PHENYL BUTYRATE COMPOUND FOR THE TREATMENT OF NEURODEGENERATIVE DISEASES

(71) Applicant: Amylyx Pharmaceuticals, Inc., Cambridge, MA (US)

(72) Inventors: Joshua Cohen, Canton, MA (US); Justin Klee, Cambridge, MA (US)

(21) Appl. No.: 18/707,761

(22) PCT Filed: Nov. 7, 2022
(86) PCT No.: PCT/US2022/049163

§ 371 Date: May 6, 2024

Related U.S. Application Data
(60) Provisional application No. 63/404,516, filed on Sep. 7, 2022.
(60) Provisional application No. 63/277,007, filed on Nov. 8, 2021.

(51) Int. Cl. A61K 31/575; A61K 31/13; A61K 31/192; A61K 31/27; A61K 31/473; A61K 31/55; A61P 25/28

(52) U.S. Cl. CPC A61K 31/575; A61K 31/13; A61K 31/192; A61K 31/27; A61K 31/473; A61K 31/55; A61P 25/28

(57) ABSTRACT

Provided herein are methods and compositions for treating a neurodegenerative disease (e.g., AD or PSP). The methods can include administering to the subject a bile acid or a pharmaceutically acceptable salt thereof and a phenylbutyrate compound.

### CROSS-REFERENCE TO RELATED APPLICATIONS

This application claims the benefit of priority to U.S. Application Nos. 63/277,007 and 63/404,516, filed on Nov. 8, 2021 and Sep. 7, 2022, respectively.

### TECHNICAL FIELD

The present disclosure generally relates to compositions and methods for treating various disorders.

### BACKGROUND

Neurodegenerative diseases of the central nervous system (CNS) cause progressive loss of neuronal structure and function and are devastating diseases for affected patients and their families. Among these neurodegenerative diseases are, for example, Multiple Sclerosis (MS), various types of tauopathies (e.g., Alzheimer's disease), Parkinson's disease, Alzheimer's disease, Huntington's disease, amyotrophic lateral sclerosis (ALS) and stroke. Due to the complexity of the CNS, many of these diseases are only poorly understood to date.

Alzheimer's disease is characterized by the loss of neurons and synapses in the cerebral cortex and atrophy in the temporal and parietal lobes. Abnormal aggregates of amyloid plaques and neurofibrillary tangles are the primary histopathological findings of AD and are the target of many clinical trials. However, recent studies suggest that amyloid reduction may be less able to halt pathology after AD has progressed beyond the stage of mild cognitive impairment (MCI). At this stage, neuronal death and inflammatory pathways may contribute to disease progression to a greater degree than amyloid or tau. This suggests that there may be patient sub-groups that may not respond to amyloid-targeted therapies, yet may benefit from therapies targeting cell death and inflammation.

### SUMMARY

In some aspects, the present disclosure provides methods of treating at least one symptom of progressive supranuclear palsy (PSP), the method comprising administering to the subject a pharmaceutically effective amount of a combination of TURSO and sodium phenylbutyrate.

In other aspects, also provided herein, are methods of treating at least one symptom of Alzheimer's disease (AD) in a human subject, the method comprising administering to the human subject a pharmaceutically effective amount of a combination of TURSO and sodium phenylbutyrate, wherein the human subject: (a) carries one or more copies of the APOEε4 allele; (b) has a cerebral spinal fluid (CSF) level of total tau of about 300 μg/mL or higher, or (c) has a CSF level of phospho-tau of about 70 μg/mL or higher. In some embodiments, prior to administration, provided herein is a step of determining whether the human subject has at least one of the characteristics of (a)-(c). In some instances, the human subject has a cerebral spinal fluid (CSF) level of total tau of about 300 μg/mL or higher. In some embodiments, the human subject has a CSF level of phospho-tau of about 70 μg/mL or higher.

In another aspect, provided herein are methods of slowing Alzheimer's disease (AD) progression in a human subject having one or more symptoms of AD, the method comprising administering to the subject a pharmaceutically effective amount of a combination of TURSO and sodium phenylbutyrate.

In another aspect, provided herein are methods increasing survival time of a human subject having one or more symptoms of Alzheimer's disease, the method comprising administering to the subject a pharmaceutically effective amount of a combination of TURSO and sodium phenylbutyrate.

In another aspect, provided herein are methods of decreasing the level of total CSF tau, decreasing the level of CSF phospho-tau, increasing CSF Aβ₁₋₄₂/Aβ₁₋₄₀, or increasing the level of CSF 8-OHDG in a human subject having one or more symptoms of Alzheimer's disease, the method comprising administering to the subject a pharmaceutically effective amount of a combination of TURSO and sodium phenylbutyrate. In some embodiments, the phospho-tau species is phospho-tau 181.

In another aspect, provided herein are methods of treating and/or preventing a tauopathy in a human subject, the method comprising administering to the human subject a pharmaceutically effective amount of a combination of TURSO and sodium phenylbutyrate. In some embodiments, the subject has a baseline CSF total tau level of about 300 μg/mL or higher. In some embodiments, the tauopathy is progressive supranuclear palsy (PSP), frontotemporal lobar degeneration (FTLD-TAU), corticobasal degeneration, Pick's disease, argyrophilic grain disease, post-encephalitic parkinsonism, chronic traumatic encephalopathy, primary age-related tauopathy, stroke, traumatic brain injury, or Alzheimer's disease. In some embodiments, the tauopathy is progressive supranuclear palsy.

In other aspects, also provided herein are methods of treating and/or preventing an amyloidosis related condition in a human subject, the method comprising administering to the human subject a pharmaceutically effective amount of a combination of TURSO and sodium phenylbutyrate.

In another aspect, provided herein are methods comprising administering to a human subject at risk for developing Alzheimer's disease a pharmaceutically effective amount of a combination of TURSO and sodium phenylbutyrate. In some embodiments, the subject is determined to be at risk for developing Alzheimer's disease by evaluating a level of a biomarker in a biological sample obtained from the subject. In some embodiments, the biomarker is total tau or phospho-tau. In some embodiments, the biological sample is CSF. In some embodiments, the subject carries one or more copies of the APOEε4 allele. In some embodiments, the subject carries one or more mutations in at least one gene selected from the group consisting of: APP, PSEN1, and PSEN2.

In another aspect, provided herein are methods decreasing the CSF levels of FABP3, neurogranin, YKL-40, or IL-15 in a human subject having one or more symptoms of Alzheimer's disease, the method comprising administering to the subject a pharmaceutically effective amount of a combination of TURSO and sodium phenylbutyrate.

In another aspect, provided herein are methods of treating at least one symptom of a neurodegenerative disease characterized by elevated total tau levels or phospho-tau levels, the method comprising administering to the subject a pharmaceutically effective amount of a combination of TURSO and sodium phenylbutyrate.

In another aspect, provided herein are methods of treating at least one symptom of a neurodegenerative disease characterized by elevated YKL-40 levels, the method comprising administering to the subject a pharmaceutically effective amount of a combination of TURSO and sodium phenylbutyrate. In some embodiments, the neurodegenerative disease is Alzheimer's disease. In some embodiments, the neurodegenerative disease is PSP. In some embodiments, the neurodegenerative disease is cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration (FTLD-TAU), corticobasal degeneration, Pick's disease, argyrophilic grain disease, post-encephalitic parkinsonism, chronic traumatic encephalopathy, primary age-related tauopathy, stroke, traumatic brain injury, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, subacute sclerosing panencephalitis, Tangle only dementia, multi-infarct dementia, or ischemic stroke.

In another aspect, provided herein are methods of decreasing the level of CSF YKL-40, decreasing the level of Ptpn1, or increasing the CSF ratio of 33 kDa tau to 55 kDa tau in a human subject having one or more symptoms of PSP, the method comprising administering to the subject a pharmaceutically effective amount of a combination of TURSO and sodium phenylbutyrate.

In some embodiments of any of the methods described herein, the TURSO and the sodium phenylbutyrate are administered once a day or twice a day. In some embodiments of any of the methods described herein, TURSO is administered to the subject at a dose of about 5 mg/kg to about 100 mg/kg. In some embodiments of any of the methods described herein, sodium phenylbutyrate is administered to the subject at a dose of about 10 mg/kg to about 400 mg/kg. In some embodiments of any of the methods described herein, the TURSO is administered at an amount of about 0.5 to about 5 grams per day. In some embodiments of any of the methods described herein, the sodium phenylbutyrate is administered at an amount of about 0.5 grams to about 10 grams per day. In some embodiments of any of the methods described herein, the methods comprise administering to the subject 1 gram of TURSO and 3 grams of sodium phenylbutyrate once a day or twice a day. In some embodiments of any of the methods described herein, the methods comprise administering to the subject 1 gram of TURSO once a day and 3 grams of sodium phenylbutyrate once a day for about 14 days or more, followed by administering to the subject about 1 gram of TURSO twice a day and 3 grams of sodium phenylbutyrate twice a day. In some embodiments of any of the methods described herein, the TURSO and the sodium phenylbutyrate are administered orally. In some embodiments of any of the methods described herein, the TURSO and the sodium phenylbutyrate are formulated as a single powder formulation.

In some embodiments of any of the methods described herein, the methods further comprise administering one or more additional therapeutic agents to the subject. In some embodiments, the therapeutic agent is tacrine, rivastigmine, galantamine, donepezil, or memantine.

In some embodiments of any of the methods described herein, the methods further comprise administering to the human subject a plurality of food items comprising solid foods or liquid foods. In some embodiments of any of the methods described herein, the human subject is about 18 years or older.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### BRIEF DESCRIPTION OF THE DRAWINGS

FIG. 1 is a bar graph showing levels of hippocampal Aβ42 when treated with a vehicle or AMX0035.

FIG. 2 is a schematic of the overall study workflow.

FIG. 3 is a chart summarizing levels of various biomarker in AD.

FIGS. 4A and 4B are schematic representations of the enrolled participants.

FIG. 5 is a chart of baseline characteristics.

FIG. 6 is a chart of baseline characteristics for various cognitive tests.

FIG. 7 is chart showing the baseline levels of biomarkers in the CSF.

FIG. 8 is a schematic showing ApoEε4 status.

FIG. 9 is graphical representation showing a summary of those that were excluded, withdrawn, or discontinued early.

FIG. 10 shows the summary of adverse events in the intent-to-treat population.

FIG. 11 is a chart showing the calculated GST for the Intent-to-Treat LZCF Population.

FIGS. 12A and 12B shows data of the secondary efficacy endpoints for the intent-to-treat (LZCF) population.

FIG. 13 shows tau and phospho-tau levels in the intent-to-treat (LZCF) population.

FIG. 14 shows data for Aβ₁₋₄₀, Aβ₁₋₄₂, and Aβ₁₋₄₂/Aβ₁₋₄₀ levels in the intent-to-treat (LZCF) population.

FIG. 15 is a bar graph showing levels of CSF 8-OHdG in the intent-to-treat (LZCF) population.

### DETAILED DESCRIPTION

Provided herein are compositions and methods for treating neurodegenerative diseases (e.g., Multiple Sclerosis (MS), various types of tauopathies (e.g., Alzheimer's disease), Parkinson's disease, Huntington's disease, amyotrophic lateral sclerosis (ALS), and stroke, etc.). Age-associated neurodegenerative diseases are characterized by specific key protein inclusions, accompanied with neuronal loss and gliosis.

A major class of neurodegenerative diseases, collectively known as tauopathies, are characterized by intra-cellular inclusions composed of abnormally-modified microtubule-binding protein, tau, at autopsy. A tauopathy is a disorder characterized by an abnormal level of tau in a cell, a tissue, or a fluid in an individual. In some cases, a tauopathy is characterized by the presence in a cell, a tissue, or a fluid of elevated (higher than normal) levels of tau or tau polypeptides and/or pathological forms of tau. For example, in some cases, a tauopathy is characterized by the presence in brain tissue and/or cerebrospinal fluid of elevated levels of tau or tau polypeptides and/or pathological forms of tau. A “higher than normal” level of tau in a cell, a tissue, or a fluid indicates that the level of tau in the tissue or fluid is higher than a normal, control level, e.g., higher than a normal, control level for an individual or population of individuals of the same age group. In other cases, a tauopathy is characterized by the presence in a cell, a tissue, or a fluid of lower than normal levels of tau. A “lower than normal” level of tau in a tissue or a fluid indicates that the level of tau in the cell, tissue, or fluid is lower than a normal, control level, e.g., lower than a normal, control level for an individual or population of individuals of the same age group. In some cases, an individual having a tauopathy exhibits one or more additional symptoms of a tauopathy (e.g., cognitive decline).

Examples of tauopathies include, but are not limited to, progressive supranuclear palsy (PSP), cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration (FTLD-TAU), corticobasal degeneration, Pick's disease, argyrophilic grain disease, post-encephalitic parkinsonism, chronic traumatic encephalopathy, primary age-related tauopathy, stroke, traumatic brain injury, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, subacute sclerosing panencephalitis, Tangle only dementia, multi-infarct dementia, ischemic stroke, and Alzheimer's disease (e.g., Irwin D J. Tauopathies as clinicopathological entities. Parkinsonism Relat Disord. 2016:22 Suppl 1 (0 1): S29-S33. doi: 10.1016/j.parkreldis.2015.09.020). Alzheimer's Disease (“AD”) results in a progressive decline of cognitive functions, including loss of declarative and procedural memory, decreased learning ability, reduced attention span, and severe impairment in thinking ability, judgment, and decision making Alzheimer's disease is characterized by the loss of neurons and synapses in the cerebral cortex and atrophy in the temporal and parietal lobes. Abnormal aggregates of amyloid plaques and neurofibrillary tangles are two of the primary histopathological findings of AD and have been the target of many recent clinical trials. However, recent studies suggest that amyloid reduction is less able to halt pathology after AD progresses beyond the stage of mild cognitive impairment (MCI). By this point, parallel neuronal death and inflammatory pathways may contribute to disease progression greater than either amyloid or tau. This suggests that there is a significant patient group that may not respond to amyloid-targeted therapies alone, yet may benefit from therapies targeting cell death and inflammation.

The terms “Alzheimer's Disease” and “AD” are used interchangeably herein, and include all of the classifications of Alzheimer's Disease known in the art. The present disclosure provides methods of treating at least one symptom of AD, methods of reducing AD disease progression; and methods of reducing the deterioration of one or more bodily functions affected by AD, maintaining one or more bodily functions affected by AD, or improving one or more bodily functions affected by AD. Also provided are methods of ameliorating one or more biomarkers that is affected in a person with AD (for example, lowering the levels of total tau and phospho-tau, which are both elevated in AD). The methods include administering a bile acid or a pharmaceutically acceptable salt thereof, and a phenylbutyrate compound.

The term “progressive supranuclear palsy” or “PSP” refers to a neurologic disorder of unknown origin that gradually destroys cells in many areas of the brain and the accumulation of abnormal aggregates of the microtubule-associated protein tau, resulting in insoluble paired helical filaments, including the gradual deterioration of neurons and glial cells in the midbrain and frontal cortex that display insoluble helical filaments of tau proteins.

PSP starts with a pre-symptomatic phase during which there is an increase in neuropathological abnormalities. Next, patients develop isolated symptoms that are suggestive of PSP (soPSP). In any of the methods described herein, PSP can be classic PSP-Richardson's syndrome (PSP-RS), PSP-Parkinsonism (PSP-P), PSP-corticobasal syndrome (PSP-CBS), PSP-progressive non-fluent aphasia (PSP-PNFA), or PSP-pure akinesia with gait freezing (PSP-PAGF) (Ling et al., J. Mov. Discord. 9 (1): 3-13, 2016). In some embodiments of any of the methods described herein, a subject can be previously diagnosed or identified as having PSP (e.g., PSP-RS, PSP-P, PSP-PNFA, or PSP-PAGF). In some embodiments of any of the methods described herein, a subject can previously be identified as having an increased risk of developing PSP (e.g., a subject having a genetically-related family member (e.g., a parent, grandparent, aunt, uncle, or sibling) that has been identified or diagnosed as having PSP) In some embodiments of any of the methods described herein, a subject can previously be identified or diagnosed as having pre-symptomatic PSP or suggestive-of-PSP.

After onset, symptoms of PSP become rapidly and progressively worse. Subjects diagnosed with PSP may become severely disabled within five years and die within six years.

Additionally, in some cases, provided herein are compositions and methods for treating amyloidosis related conditions. Amyloid fibrils are protein polymers comprising identical monomer units (homopolymers). Functional amyloids play a beneficial role in a variety of physiologic processes (eg, long-term memory formation, gradual release of stored peptide hormones). Amyloidosis is a clinical disorder caused by extracellular and/or intracellular deposition of insoluble abnormal amyloid fibrils that alter the normal function of tissues. Only 10% of amyloidosis deposits consist of components such as glycosaminoglycans (GAGs), apolipoprotein-E (apoE), and serum amyloid P-component (SAP), while nearly 90% of the deposits consist of amyloid fibrils that are formed by the aggregation of misfolded proteins. These proteins either arise from proteins expressed by cells at the deposition site (localized), or they precipitate systemically after production at a local site (systemic). In humans, about 23 different unrelated proteins are known to form amyloid fibrils in vivo.

Many mechanisms of protein function contribute to amyloidogenesis, including “nonphysiologic proteolysis, defective or absent physiologic proteolysis, mutations involving changes in thermodynamic or kinetic properties, and pathways that are yet to be defined.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art.

### Diagnosis and Subject Selection

### Alzheimer's Disease

In one aspect, the present disclosure provides methods of treating at least one symptom of AD in a human subject. Also provided herein are methods of slowing AD disease progression (e.g., reducing the AD disease progression rate); methods of treating dementia or mild cognitive impairment (MCI) (e.g. dementia or MIC due to AD); and methods of reducing the progressive decline of cognitive functions, including loss of declarative and procedural memory, decreased learning ability, reduced attention span, and severe impairment in thinking ability, judgment, and decision making. Also provided are methods of increasing survival time of a human subject having one or more symptoms of AD. Also provided are methods of ameliorating one or more biomarkers that is affected in a human subject with AD (for example, lowering the levels of total tau and phospho-tau, both may be elevated in AD). Any of the methods described herein can include administering to the subject a bile acid or a pharmaceutically acceptable salt thereof (e.g., any of the bile acid or a pharmaceutically acceptable salt thereof described herein or known in the art) and a phenylbutyrate compound (e.g., any of the phenylbutyrate compound described herein or known in the art).

Any of the human subjects in the methods described herein may exhibit one or more symptoms associated with AD, or have been diagnosed with AD. In some embodiments, the subjects may be suspected as having AD, and/or at risk for developing AD.

Some embodiments of any of the methods described herein can further include determining that a human subject has or is at risk for developing AD, diagnosing a human subject as having or at risk for developing AD, or selecting a human subject having or at risk for developing AD.

In some embodiments of any of the methods described herein, the human subject has shown one or more symptoms of AD for about 24 months or less (e.g., about 23, 22, 21, 20, 19, 18, 17, 16, 15, 14, 13, 12, 11, 10, 9, 8, 7, 6, 5, 4, 3, 2, 1 month, or 1 week or less). In some embodiments, the subject has shown one or more symptoms of AD for about 36 months or less (e.g., about 35, 34, 33, 32, 31, 30, 29, 28, 27, 26, or 25 months or less).

In some instances, the human subject has been diagnosed with AD. For example, the subject may have been diagnosed with AD for about 24 months or less (e.g., about 23, 22, 21, 20, 19, 18, 17, 16, 15, 14, 13, 12, 11, 10, 9, 8, 7, 6, 5, 4, 3, 2, or 1 month or less). For example, the subject may have been diagnosed with AD for 1 week or less, or on the same day that the presently disclosed treatments are administered. The subject may have been diagnosed with AD for longer than about 24 months (e.g., longer than about 28, 32, 36, 40, 44, 48, 52, 56, 60, 64, 68, 72, 76, or 80 months). Methods of diagnosing AD are known in the art. For example, the subject can be diagnosed based on clinical history, family history, physical or neurological examinations. The subject can be confirmed or identified, e.g. by a healthcare professional, as having AD. Multiple parties may be included in the process of diagnosis. For example, where samples are obtained from a subject as part of a diagnosis, a first party can obtain a sample from a subject and a second party can test the sample. In some embodiments of any of the human subjects described herein, the subject is diagnosed, selected, or referred by a medical practitioner (e.g., a general practitioner).

Generally, diagnosis of AD is well known in the art (see, e.g., Alzheimer's Disease Diagnostic Guidelines issued by the NIH's National Institute on Aging, https://www.nia.nih.gov/health/alzheimers-disease-diagnostic-guidelines, which is incorporated herein by reference). In some embodiments, the subject has a diagnosis of “Probable Alzheimer's Disease” or “Mild Cognitive Impairment” with a primarily amnestic presentation (deficit in learning and recall of recently learned information). In some instances, the diagnosis of, “Probable Alzheimer's Disease” or “Mild Cognitive Impairment,” is based upon a score received from a cognitive test (e.g., ADAS-Cog, MoCA, DSRS, MADCOMS, FAQ, or NPI-Q) accompanied by the presence of one or more biomarker (amyloid PET (i.e., presence of amyloid plaques, CSF AD biomarkers (as described in detail below and in Example 1), FDG-PET, or vMRI) supporting that the syndrome is likely due to AD pathology.

### ADAS-Cog

The ADAS-Cog is validated and widely used as a primary cognitive outcome measure in AD pharmacotherapy studies. This is a psychometric instrument that evaluates memory (immediate and delayed word recall, word recognition), attention (number cancellation), reasoning (following commands), language (naming, comprehension), orientation, ideational praxis (placing letter in envelope) and constructional praxis (copying geometric designs), and executive functioning (maze completion). Scoring is in the range of 0 to 90 with a higher score indicating greater impairment.

### MoCA

Montreal Cognitive Assessment (MoCA) is a commonly utilized questionnaire in clinical trials and research settings to measure levels of cognitive impairment. The MoCA measures five areas of cognitive function: orientation, visuospatial, attention and calculation, recall, and language. The MoCA takes approximately 10 minutes to complete. In some instances, an AD patient has a baseline MoCA score between 8-30, e.g., greater than 8.

### DSRS

The DSRS is a brief 12-item questionnaire administered to an informant that assesses a subjects' functional abilities⁴⁴ and offers a global characterization of everyday activities that may be impacted by neurodegenerative disease. The DSRS is designed in a multi-choice format with strong concurrent validity and parallel content to material covered on the Clinical Dementia Rating Scale (CDR), a commonly employed dementia staging instrument (Moelter, S. T., Glenn, M. A., Xie, S. X., Chittams, J., Clark, C. M., Watson, M., & Arnold, S. E. (2015). The Dementia Severity Rating Scale predicts clinical dementia rating sum of boxes scores. Alzheimer Dis Assoc Disord, 29 (2), 158-160. doi: 10.1097/WAD.0000000000000031). The DSRS is a highly reliable scale with an intra-class correlation of >90% for interrater reliability and Cronbach's alpha >0.70 for internal consistency (Rikkert, M. G., Tona, K. D., Janssen, L., Burns, A., Lobo, A., Robert, P., . . . Waldemar, G. (2011). Validity, reliability, and feasibility of clinical staging scales in dementia: a systematic review. Am J Alzheimers Dis Other Demen, 26 (5), 357-365. doi: 10.1177/1533317511418954), and has been shown to accurately discriminate between cognitive healthy individuals and dementia subjects of varying severity (Clark, C. M., & Ewbank, D. C. (1996). Performance of the dementia severity rating scale: a caregiver questionnaire for rating severity in Alzheimer disease. Alzheimer Dis Assoc Disord, 10(1), 31-39; Mitchell, J. C., Dick, M. B., Wood, A. E., Tapp, A. M., & Ziegler, R. (2015). The utility of the Dementia Severity Rating Scale in differentiating mild cognitive impairment and Alzheimer disease from controls. Alzheimer Dis Assoc Disord, 29 (3), 222-228. doi: 10.1097/WAD.0000000000000057). Further, the DSRS allows for a broad range of scores (total score 0-54) making it suitable to quantify a wide range of functional impairment without being hampered by floor effects seen in more advanced disease, while also making it sensitive to detecting incremental change in functional ability over time (Xie, S. X., Ewbank, D. C., Chittams, J., Karlawish, J. H., Arnold, S. E., & Clark, C. M. (2009). Rate of decline in Alzheimer disease measured by a Dementia Severity Rating Scale. Alzheimer Dis Assoc Disord, 23 (3), 268-274. doi: 10.1097/WAD.0b013e318194a324). The DSRS takes about 5 minutes to administer, requires minimal rater training, and can be administered over the phone to study subjects if required.

### MADCOMS

ADAS-Cog 14 is not specifically targeted to the mild/moderate stage of AD. A mild/moderate AD composite scale (MADCOMS) was previously optimized for the two distinct groups, mild AD (baseline MMSE 20-26) and moderate AD (baseline MMSE 14-19). The weighted composite was derived using PLS regression from ADAS-Cog, MMSE, and CDR individual items (S.Hendrix ADPD 2021).

$$Moderate MADCOM = Comprehension * 0.36390157 + Word Finding * 0.10931155 + Ideational Praxis * 0.42535667 + Naming Objects * 0.65626894 + Word Recognition * 0.05159097 + Word Recall * 1.0698506 + Spoken Language * 0.3019936 + Home and Hobbies * 0.66529282 + Memory * 0.12277257 - Orientation to Place * 0.23001218 - Spell Backward * 0.07980965 - Language and Praxis * 0.18954955$$

$$Mild MADCOM = Word Finding * 0.39065568 + Word Recall * 1.14084544 + Spoken Language * 1.0989559 + Personal Care * 0.60865765 + Community Affairs * 0.15706995 + Judgment * 1.40920029 - Orientation to Time * 0.27596627$$

### FAQ

The FAQ is a brief informant-administered rating scale used to determine a subjects' level of functional independence when performing a range of instrumental activities of daily living (IADLs), with repeat assessments useful for monitoring performance in these areas over time⁴⁷ (see below for the scale). The FAQ total score (ranging from 0-30) reflects the sum of ordinal ratings (0=fully independent, 1=has difficulty but does by self, 2=requires assistance, and 3=dependent) across ten items assessing a variety of functional activities (i.e., preparing a balanced meal, financial management skills, and shopping), with higher scores indicating increasing levels of dependence. For activities not normally undertaken by a person, a score of 1 was assigned if the informant believed the subject would be unable to complete the task if required, or a score of 0 was assigned if the informant believed the subject could successfully carry out the task if needed. Overall, the FAQ is a sensitive marker of functional impairment among individuals with varying dementia severity (see, e.g., Castilla-Rilo, J., Lopez-Arrieta, J., Bermejo-Pareja, F., Ruiz, M., Sanchez-Sanchez, F., & Trincado, R. (2007). Instrumental activities of daily living in the screening of dementia in population studies: a systematic review and meta-analysis. Int J Geriatr Psychiatry, 22 (9), 829-836. doi: 10.1002/gps.1747), and has been shown to differentiate mild cognitive impairment from early Alzheimer's Disease with 80% sensitivity and 87% specificity (see, e.g., Teng, E., Becker, B. W., Woo, E., Knopman, D. S., Cummings, J. L., & Lu, P. H. (2010). Utility of the functional activities questionnaire for distinguishing mild cognitive impairment from very mild Alzheimer disease. Alzheimer Dis Assoc Disord, 24 (4), 348-353. doi: 10.1097/WAD.0b013e3181e2fc84). The FAQ demonstrates high reliability (exceeding 0.90), takes about 5 minutes to complete, and requires limited rater training to administer (see, e.g., (Pfeffer, R. I., Kurosaki, T. T., Harrah, C. H., Jr., Chance, J. M., & Filos, S. (1982). Measurement of functional activities in older adults in the community. J Gerontol, 37 (3), 323-329).

### NPI-Q

The Neuropsychiatric Inventory (NPI) measures dementia-related behavioral symptoms and was used to assess changes in psychological status. There are several versions of the NPI including the NPI-Questionnaire (NPI-Q), NPI-Clinician (NPI-C) and the NPI-Nursing Home (NPI-NH). All examine 12 sub-domains of behavioral functioning including: hallucinations, delusions, agitation, dysphoria, anxiety, euphoria, apathy, disinhibition, irritability, aberrant motor activity, eating abnormalities, and night-time behavioral alternations.

### A/T/N System

In some cases, the “A/T/N′ system” is used to classify patients with AD based on biomarkers. In this system, 7 major AD biomarkers are divided into 3 binary categories based on the nature of the pathophysiology that each measures. “A” refers to the value of a β-amyloid biomarker (amyloid PET or CSF Aβ42); “T,” the value of a tau biomarker (CSF phospho tau, or tau PET); and “N,” biomarkers of neurodegeneration or neuronal injury (¹⁸F fluorodeoxyglucose-PET, structural MRI, or CSF total tau). Each biomarker category is rated as positive or negative. An individual score might appear as A+/T+/N−, or A+/T−/N−. See, e.g., Jack C R Jr, Bennett D A, Blennow K, Carrillo M C, Feldman H H, Frisoni G B, Hampel H, Jagust W J, Johnson K A, Knopman D S, Petersen R C, Scheltens P, Sperling R A, Dubois B. A/T/N: An unbiased descriptive classification scheme for Alzheimer disease biomarkers. Neurology. 2016 Aug. 2; 87 (5): 539-47 and Kalmady et al. 2014).

### Neuroimaging

Neuroimaging examinations may also be utilized to diagnose a subject who is at risk or is suffering from AD. Methods for measuring hippocampal volume, grey matter, average cortical thickness, number of white matter lesions, white matter lesion volume, ventricular volume include: medial temporal lobe atrophy as assessed with magnetic resonance imaging (MRI) and reduced glucose metabolism in temporoparietal regions on functional neuroimaging with ¹⁸F-fluorodeoxyglucose-positron emission tomography (FDG-PET), single photon emission computed tomography (SPECT), diffusion tensor imaging (DIT), Amyloid imaging, computed tomography (CT). See, e.g., Ferreira L K, Busatto G F. Neuroimaging in Alzheimer's disease: current role in clinical practice and potential future applications. Clinics (Sao Paulo). 2011; 66 Suppl 1 (Suppl 1): 19-24. doi: 10.1590/s1807-59322011001300003

### CSF AD Biomarkers

In some embodiments, a subject may be chosen based on the levels of certain CSF biomarkers. The concentration of one or more biomarkers such as, total tau (t-tau), phospho-tau 181 (p-tau 181), neurofilament-light (NfL), Ubiquitin carboxyl-terminal hydrolase L1 (UCHL1)/PGP9.5, Glial fibrillary acidic protein (GFAP), 8-hydoxy-2′-deoxyguanosine (8-OHdG), Soluble insulin receptor (sIR) may be elevated in the CSF of AD patients. Aβ₁₋₄₂, Aβ₁₋₄₂/Aβ₁₋₁₀, and leptin may be reduced in the CSF of AD patients. 24-hydroxycholesterol (24-OHC) may be elevated in early AD, and reduced in advanced AD

Any of the above mentioned biomarkers can be detected e.g., in the cerebrospinal fluid, plasma and/or serum using known methods in the art, (See e.g., Blennow K, Mattsson N, Schöll M, Hansson O, Zetterberg H. Amyloid biomarkers in Alzheimer's disease. Trends Pharmacol Sci 2015; 36:297-309. https://doi.org/https://doi.org/10.1016/j tips.2015.03.002; Mattsson N, Insel P S, Palmqvist S, Portelius E, Zetterberg H, Weiner M, et al. Cerebrospinal fluid tau, neurogranin, and neurofilament light in Alzheimer's disease. EMBO Mol Med 2016; 8:1184-96. https://doi.org/https://doi.org/10.15252/emmm.201606540; Gaetani L, Blennow K, Calabresi P, Di Filippo M, Parnetti L, Zetterberg H. Neurofilament light chain as a biomarker in neurological disorders J Neurol Neurosurg &amp; Amp; Psychiatry 2019; 90:870 LP-881. https://doi.org/10.1136/jnnp-2018-320106; Constantinescu R, Krýsl D, Bergquist F, Andrén K, Malmeström C, Asztely F, et al. Cerebrospinal fluid markers of neuronal and glial cell damage to monitor disease activity and predict long-term outcome in patients with autoimmune encephalitis. Eur J Neurol 2016:23:796-806. https://doi.org/https://doi.org/10.1111/ene.12942; Hall S, Ohrfelt A, Constantinescu R, Andreasson U, Surova Y, Bostrom F, et al. Accuracy of a Panel of 5 Cerebrospinal Fluid Biomarkers in the Differential Diagnosis of Patients With Dementia and/or Parkinsonian Disorders. Arch Neurol 2012; 69:1445-52. https://doi.org/10.1001/archneurol.2012.1654; Petersen A, Gerges N Z. Neurogranin regulates CaM dynamics at dendritic spines. Sci Rep 2015; 5:11135. https://doi.org/10.1038/srep11135; Portelius E, Zetterberg H, Skillbäck T, Törnqvist U, Andreasson U, Trojanowski J Q, et al. Cerebrospinal fluid neurogranin: relation to cognition and neurodegeneration in Alzheimer's disease. Brain 2015; 138:3373-85. https://doi.org/10.1093/brain/awv267; Liu W, Lin H, He X, Chen L, Dai Y, Jia W, et al. Neurogranin as a cognitive biomarker in cerebrospinal fluid and blood exosomes for Alzheimer's disease and mild cognitive impairment. Transl Psychiatry 2020; 10:125. https://doi.org/10.1038/s41398-020-0801-2; Isobe C, Abe T, Terayama Y. Levels of reduced and oxidized coenzyme Q-10 and 8-hydroxy-2′-deoxyguanosine in the CSF of patients with Alzheimer's disease demonstrate that mitochondrial oxidative damage and/or oxidative DNA damage contributes to the neurodegenerative process. J Neurol 2010; 257:399-404. https://doi.org/10.1007/s00415-009-5333-x; Gerena Y, Menéndez-Delmestre R, Skolasky R L, Hechavarria R M, Pérez S, Hilera C, et al. Soluble insulin receptor as a source of insulin resistance and cognitive impairment in HIV-seropositive women. J Neurovirol 2015; 21:113-9. https://doi.org/10.1007/s13365-014-0310-2; Gerena Y, Menendez-Delmestre R, Delgado-Nieves A, Vélez J, Méndez-Alvarez J, Sierra-Pagan J E, et al. Release of Soluble Insulin Receptor From Neurons by Cerebrospinal Fluid From Patients With Neurocognitive Dysfunction and HIV Infection. Front Neurol 2019; 10:285. https://doi.org/10.3389/fneur.2019.00285; Hughes T M, Rosano C, Evans R W, Kuller L H. Brain cholesterol metabolism, oxysterols, and dementia. J Alzheimers Dis 2013; 33:891-911. https://doi.org/10.3233/JAD-2012-121585; Papassotiropoulos A, Lütjohann D, Bagli M, Locatelli S, Jessen F, Rao M L, et al. Plasma 24S-hydroxycholesterol: a peripheral indicator of neuronal degeneration and potential state marker for Alzheimer's disease. Neuroreport 2000; 11:1959-62. https://doi.org/10.1097/00001756-200006260-00030; Cuadrado E, Rosell A, Penalba A, Slevin M, Alvarez-Sabin J, Ortega-Aznar A, et al. Vascular MMP-9/TIMP-2 and neuronal MMP-10 up-regulation in human brain after stroke: a combined laser microdissection and protein array study. J Proteome Res 2009; 8:3191-7. https://doi.org/10.1021/pr801012x; Duits F H, Hernandez-Guillamon M, Montaner J, Goos J D C, Montañola A, Wattjes M P, et al. Matrix Metalloproteinases in Alzheimer's Disease and Concurrent Cerebral Microbleeds. J Alzheimers Dis 2015; 48:711-20. https://doi.org/10.3233/JAD-143186.). Commercialized detection assays can also be used.

In some instances, the ratio of a subject in any of the methods described herein may be identified by obtaining and measuring a ratio of Aβ₁₋₄₂/Aβ₁₋₄₀ (as measured in the CSF). The Aβ₁₋₄₂/Aβ₁₋₄₀ ratio is a key marker in Alzheimer's and decreases in the CSF in patients with Alzheimer's Disease. Studies have shown that the CSF Aβ₁₋₄₂/Aβ₁₋₄₀ is a more reliable indicator of AD than other typical AD biomarkers. It has also been suggested that the ratio can be used to differentiate between types of dementia. See, e.g., Hansson, O., Lehmann, S, Otto, M. et al. Advantages and disadvantages of the use of the CSF Amyloid β (Aβ) 42/40 ratio in the diagnosis of Alzheimer's Disease. Alz Res Therapy 11, 34 (2019). https://doi.org/10.1186/s13195-019-0485-0 and James D. Doecke, Virginia Pérez-Grijalba, Noelia Fandos, Christopher Fowler, Victor L. Villemagne, Colin L. Masters, Pedro Pesini, Manuel Sarasa, for the AIBL Research Group, “Total Aβ42/Aβ40 ratio in plasma predicts amyloid-PET status, independent of clinical AD diagnosis,” Neurology April 2020, 94 (15) e1580-e1591; DOI: 10.1212/WNL.0000000000009240, incorporated herein by reference

Subjects in the methods described herein may have a CSF total tau level of about 100 pg/mL or higher. In some embodiments, the subjects have a CSF total tau level of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher).

Subjects in the methods described herein may have a CSF phospho-tau (e.g. phospho-tau 181, phospho-tau 199, and/or phosphor-tau 231) level of about 30 pg/mL or higher. In some embodiments, the subjects have a CSF phospho-Tau (e.g. phosphor-tau 181) level about 70 pg/mL or higher (e.g., about 75, 100, 125, 150, 175, or 200 pg/mL or higher).

Subjects in the methods described herein may have a CSF Aβ₁₋₄₂ level of about 1000 pg/mL or lower (e.g., about 500 pg/mL or lower). In some embodiments, the subjects have a CSF Aβ₁₋₄₂ level of about 500 pg/mL or lower (e.g., about 450, 400, 350, 300, 250, 200, 150, 100, 50, or 25 pg/mL, or lower).

Subjects in the methods described herein may have a ratio of CSF total tau to CSF Aβ₁₋₄₂ (i.e., total tau/Aβ₁₋₄₂) of about 0.5 or higher. In some embodiments, the ratio may be 0.9 or higher (e.g., 1.0, 1.1, 12, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0, or higher).

A subject may also be identified as having AD, or at risk for developing AD, based on genetic analysis. Researchers have not found a specific gene that directly causes late-onset Alzheimer's disease. However, having a genetic variant of the apolipoprotein E (APOE) gene on chromosome 19 may increase a person's risk. The APOE gene is involved in making a protein that helps carry cholesterol and other types of fat in the bloodstream. APOEε4 increases risk for Alzheimer's disease and is also associated with an earlier age of disease onset. Having one or two APOEε4 alleles increases the risk of developing Alzheimer's. About 25 percent of people carry one copy of APOEε4, and 2 to 3 percent carry two copies. APOEε4 is called a risk-factor gene because it increases a person's risk of developing the disease. APOEε2 is relatively rare (leads to an amino acid change of arginine to cysteine at position 158) and may provide some protection against the disease. APOEε3, the most common allele, is believed to play a neutral role in the disease-neither decreasing nor increasing risk. See, for instance, Chu L W. Hong Kong Med J. 2012; 18:228-237; Belloy M E, Napolioni V, Greicius M D. A Quarter Century of APOE and Alzheimer's Disease: Progress to Date and the Path Forward. Neuron. 2019 Mar. 6; 101 (5): 820-838; Reiman, E. M., Arboleda-Velasquez, J. F., Quiroz, Y. T. et al. Exceptionally low likelihood of Alzheimer's dementia in APOEε2 homozygotes from a 5,000-person neuropathological study. Nat Commun 11, 667 (2020); Shinohara M, Kanekiyo T, Tachibana M, Kurti A, Shinohara M, Fu Y, Zhao J, Han X, Sullivan P M, Rebeck G W, Fryer J D, Heckman M G, Bu G. APOEε2 is associated with longevity independent of Alzheimer's disease. Elife. 2020 Oct. 19; 9: e62199, which are incorporated herein by reference.

Early-onset Alzheimer's disease is rare, representing less than 10 percent of all people with Alzheimer's. It typically occurs between a person's 30s and mid-60s. Some cases are caused by an inherited change in one of three genes. The three single-gene mutations associated with early-onset Alzheimer's disease are: (1) Amyloid precursor protein (APP) on chromosome 21; (2) Presenilin 1 (PSEN1) on chromosome 14; or (3) Presenilin 2 (PSEN2) on chromosome 1. Mutations in these genes result in the production of abnormal proteins that are associated with the disease. Each of these mutations plays a role in the breakdown of APP. This breakdown is part of a process that generates harmful forms of amyloid plaques, a hallmark of Alzheimer's disease. Genetic variants associated with AD can affect the AD progression rate in a subject, the pharmacokinetics of the administered compounds in a subject, and/or the efficacy of the administered compounds for a subject

Baseline characteristics of AD patients are known in the art (see e.g., Mintun M A, Donanemab in Early Alzheimer's Disease, N Engl J Med. 2021 May 6; 384 (18): 1691-1704; Belloy M E, Napolioni V, Greicius M D. A Quarter Century of APOE and Alzheimer's Disease: Progress to Date and the Path Forward. Neuron. 2019 Mar. 6; 101 (5): 820-838; Reiman, E. M., Arboleda-Velasquez, J. F., Quiroz, Y. T. et al. Exceptionally low likelihood of Alzheimer's dementia in APOEε2 homozygotes from a 5,000-person neuropathological study. Nat Commun 11, 667 (2020); Shinohara M, Kanekiyo T, Tachibana M, Kurti A, Shinohara M, Fu Y, Zhao J, Han X, Sullivan P M, Rebeck G W, Fryer J D, Heckman M G, Bu G. APOEε2 is associated with longevity independent of Alzheimer's disease Elife. 2020 Oct. 19; 9.e62199, Trombetta B A, Carlyle B C, Koenig A M, Shaw L M, Trojanowski J Q, Wolk D A, Locascio J J, Arnold S E. The technical reliability and biotemporal stability of cerebrospinal fluid biomarkers for profiling multiple pathophysiologies in Alzheimer's disease. PLOS One. 2018; and Vasunilashom S M, Ngo L H, Dillon S T, Fong T G, Carlyle B C, Kivisakk P, Trombetta B A, Vlassakov K V, Kunze L J, Arnold S E, Xie Z, Inouye S K, Libermann T A, Marcantonio E R; RISE Study Group. Plasma and cerebrospinal fluid inflammation and the blood-brain barrier in older surgical patients: the Role of Inflammation after Surgery for Elders (RISE) study J Neuroinflammation. 2021 Apr. 30; 18 (1): 103 all of which are incorporated herein by reference).

### Inflammation

Neuroinflammation is also involved in the complex cascade leading to AD pathology and symptoms. Considerable pathological and clinical evidence documents immunological changes associated with AD, including increased pro-inflammatory cytokine concentrations in the blood and cerebrospinal fluid. Whether these changes may be a cause or consequence of AD remains to be fully understood, but inflammation within the brain, including increased reactivity of the resident microglia towards amyloid deposits, has been implicated in the pathogenesis and progression of AD. A subject may also be identified as having AD, or at risk for developing AD, based on the presence of neuroinflammation (see, e.g., Chu L W Hong Kong Med J. 2012; 18:228-237. 2. Newcombe E A, et al. Neuroinflammation. 2018; 15 (1): 276. doi: 10.1186/s12974-018-1313-3)

### Misfolded Proteins

AD is considered a protein misfolding disease due to the accumulation of abnormally folded amyloid beta (AB) protein in the brain. Amyloid beta is a short peptide that is an abnormal proteolytic byproduct of the transmembrane protein amyloid-beta precursor protein (APP), whose function is unclear but thought to be involved in neuronal development. The presenilins are components of proteolytic complex involved in APP processing and degradation.

Amyloid beta monomers are soluble and contain short regions of beta sheet and polyproline II helix secondary structures in solution, though they are largely alpha helical in membranes; however, at sufficiently high concentration, they undergo a dramatic conformational change to form a beta sheet-rich tertiary structure that aggregates to form amyloid fibrils. These fibrils and oligomeric forms of Aβ deposit outside neurons in formations known as senile plaques. There are different types of plaques, including the diffuse, compact, cored or neuritic plaque types, as well as Aβ deposits in the walls of small blood vessel walls in the brain called cerebral amyloid angiopathy. See, e.g., Chu L W. Hong Kong Med J. 2012; 18:228-237. 2. Newcombe E A, et al. Neuroinflammation. 2018; 15 (1): 276. doi: 10.1186/s12974-018-1313-3). Accordingly, misfolded proteins might also be useful in identifying subjects who may have AD, or at risk for developing AD.

### Mitochondrial Metabolic Dysfunction and Oxidative Stress

Mitochondrial dysfunction is widespread in neurodegenerative disease. In Alzheimer's disease, the mitochondrial membrane potential of cells is markedly reduced, glucose metabolism by the mitochondria is impaired, and the permeability of the mitochondria is increased. Mitochondria have been observed to mediate multiple apoptotic pathways resulting in neuronal death in Alzheimer's disease. See, e.g., Swerdlow R H. J Alzheimers Dis. 2018; 62 (3): 1403-1416 and Chu L W. Hong Kong Med J. 2012; 18:228-237; Tonnies E and Trushina E. J Alzheimers Dis. 2017; 57 (4): 1105-1121).

PINK 1 and Parkin are both mitochondrial quality control proteins. Mutations or lack of these proteins is strongly linked to Parkinson's disease. MPTP, a molecule used to induce permanent symptoms of Parkinson's, acts through the disruption of complex I of the mitochondria, causing mitochondrial dysfunction, alteration of the redox state of the cell, and apoptosis.

It has been directly shown in cell culture that the mutant Huntingtin gene and its resultant protein, thought to be the primary mediator of Huntington's disease, results in a loss of membrane potential and decreased expression of critical oxidative phosphorylation genes in the mitochondria. Huntington's disease pathology has also been linked to a decrease in the number of mitochondria present in the central nervous system.

Mitochondrial dyslocalization, energy metabolism impairment, and apoptotic pathways are thought to mediate Amyotrophic lateral sclerosis. Mitochondria from affected tissues have also been shown to overproduce reactive oxygen metabolites and leak them to the cytosol.

In many neurodegenerative diseases, mitochondria overproduce free radicals, cause a reduction in energy metabolism, have increased permeability, have decreased membrane potential, have decreased antioxidants, leak metal ions into the cell, alter the redox state of the cell, and lead the cell down pro-apoptotic pathways. A need therefore exists for agents that can alter and reduce mitochondrial dysfunction mechanisms. In some instances, signs of mitochondrial/metabolic dysfunction and oxidative stress may be useful in in diagnosing and selecting subjects who may have AD or are at risk for developing AD.

### Other Factors Useful for Diagnosis Subject Selection for AD

Vascular disease (e.g., Sweeny M D, et al. Alzheimers Dement. 2019; 15 (1): 158-167), synaptic activity or neurotransmitter activity (see e.g., Tonnies E and Trushina E. J Alzheimers Dis. 2017; 57 (4): 1105-1121) may also be useful in diagnosing and selecting subjects who may have AD or are at risk for developing AD.

Having a mutation in any of the AD-associated genes described herein, carrying one or more copies of APOEε4 allele, or presenting with any of the biomarkers described herein may suggest that a subject is at risk for developing AD. Such subjects can be treated with the methods provided herein for preventative and prophylaxis purposes.

### Progressive Supranucelar Palsy

In one aspect, the present disclosure provides methods of treating at least one symptom of PSP in a human subject. Also provided herein are methods of slowing PSP disease progression (e.g., reducing the PSP disease progression rate); and methods of reducing the progressive decline of cognitive functions, including loss of declarative and procedural memory, decreased learning ability, reduced attention span, and severe impairment in thinking ability, judgment, and decision making. Also provided are methods of increasing survival time of a human subject having one or more symptoms of PSP. Also provided are methods of ameliorating one or more biomarkers that is affected in a human subject with PSP (for example, lowering the levels of total tau and phospho-tau, both may be elevated in PSP or lowering the levels of YKL-40, which may also be elevated in PSP). Any of the methods described herein can include administering to the subject a bile acid or a pharmaceutically acceptable salt thereof (e.g., any of the bile acid or a pharmaceutically acceptable salt thereof described herein or known in the art) and a phenylbutyrate compound (e.g., any of the phenylbutyrate compound described herein or known in the art).

Any of the human subjects in the methods described herein may exhibit one or more symptoms associated with PSP, or have been diagnosed with PSP. In some embodiments, the subjects may be suspected as having PSP, and/or at risk for developing PSP.

Some embodiments of any of the methods described herein can further include determining that a human subject has or is at risk for developing PSP, diagnosing a human subject as having or at risk for developing PSP, or selecting a human subject having or at risk for developing PSP.

In some embodiments of any of the methods described herein, the human subject has shown one or more symptoms of PSP for about 24 months or less (e.g., about 23, 22, 21, 20, 19, 18, 17, 16, 15, 14, 13, 12, 11, 10, 9, 8, 7, 6, 5, 4, 3, 2, 1 month, or 1 week or less). In some embodiments, the subject has shown one or more symptoms of PSP for about 36 months or less (e.g., about 35, 34, 33, 32, 31, 30, 29, 28, 27, 26, or 25 months or less).

In some instances, the human subject has been diagnosed with PSP. For example, the subject may have been diagnosed with PSP for about 24 months or less (e.g., about 23, 22, 21, 20, 19, 18, 17, 16, 15, 14, 13, 12, 11, 10, 9, 8, 7, 6, 5, 4, 3, 2, or 1 month or less). For example, the subject may have been diagnosed with PSP for 1 week or less, or on the same day that the presently disclosed treatments are administered. The subject may have been diagnosed with PSP for longer than about 24 months (e.g., longer than about 28, 32, 36, 40, 44, 48, 52, 56, 60, 64, 68, 72, 76, or 80 months). Methods of diagnosing PSP are known in the art. For example, the subject can be diagnosed based on clinical history, family history, physical or neurological examinations. The subject can be confirmed or identified, e.g. by a healthcare professional, as having PSP. Multiple parties may be included in the process of diagnosis. For example, where samples are obtained from a subject as part of a diagnosis, a first party can obtain a sample from a subject and a second party can test the sample. In some embodiments of any of the human subjects described herein, the subject is diagnosed, selected, or referred by a medical practitioner (e.g., a general practitioner).

Generally, diagnosis of PSP is known in the art. Symptoms of PSP usually first appear at the age of 60 and worsen until death. People with PSP commonly die from pneumonia, choking or other complications caused by the loss of functional brain cells, resulting in loss of autonomic and motor function (e.g. the ability to swallow).

Signs and symptoms of PSP include movement, cognitive and psychiatric disorders. Voluntary movement can be impaired in PSP and include pseudobulbar palsy (i.e. inability to control facial movements), bradykinesia (i.e. slow or abnormal muscle movement), neck and trunk rigidity, impaired gait, impaired balance, posture instability and difficulty with speech and swallowing. Individuals who become unable to swallow food can be fitted with a feeding tube to provide nutrition. A most obvious, outward sign of the disease is an inability to coordinate and move the eyes normally, resulting in a vertical gaze palsy. Cognitive impairments include loss of executive functions (e.g. attention control, inhibitory control, working memory, cognitive flexibility, reasoning, problem solving and planning) and diminished fluency. Associated psychiatric symptoms include depression, feelings of irritability, sadness or apathy, insomnia, fatigue and loss of energy.

Progressive supranuclear palsy can be difficult to diagnose because signs and symptoms are similar to those of Parkinson's disease. Those of skill in the art may distinguish PSP from Parkinson's based on the lack of tremors, a lot of unexplained falls, little to no response to Parkinson's medications, and/or difficulty moving eyes, particularly downward.

### MDS PSP Diagnostic Criteria

In some embodiments, a subject can be identified as having PSP using the MDS PSP Diagnostic Criteria (as described in, e.g., Hoglinger et al., Mov. Disord. 31:644-652, 2016). The diagnostic criteria addresses four functional domains (ocular motor dysfunction, postural instability, akinesia, and cognitive dysfunction) as clinical predictors of PSP.

### Progressive Supranuclear Palsy Rating Scale

Some embodiments of any of the methods described herein can include monitoring the progression of PSP in the subject, e.g., by assessing the severity of PSP in the subject over time, e.g., using the Progressive Supranuclear Palsy Rating Scale (PSPRS) (e.g., as described in Golbe et al., Brain 130 (6): 1552-1565, 2007). The PSPRS evaluates subjects according to their ability to perform daily activities, behavior, bulbar function, ocular motor function, limb motor function, and gait.

### Genetic Alterations

In some embodiments, a subject can be identified as being at increased risk of developing PSP or identified as having PSP (e.g., any of the types of PSP described herein), e.g., at least in part, by detecting a genetic alteration in a gene encoding the microtubule-associated protein tau (MAPT) (e.g., any of the inversion polymorphisms in the MAPT gene, any of the haplotype-specific polymorphisms in the MAPT gene, the rare-coding MAPT variant (A152T), or mutations that enhance splicing of exon 10 in the MAPT gene described, e.g., in Hoglinger et al., Nature Genet. 43:699-705, 2011, and Hinz et al., Cold Spring Harb. Perspect Biol.). Non-limiting examples of genetic alterations in a gene encoding MAPT include mutations that result in the production of MAPT protein that include one or more point mutations of: S285R, L284R, P301L, and G303V. Additional specific genetic mutations in a gene encoding MAPT protein that can be used to identify a subject as having an increased risk of developing PSP or can be used to identify a subject as having PSP (e.g., any of the types of PSP described herein) are described in, e.g., Boxer et al., Lancet 16:552-563, 2017.

### Scanning and Neuroimaging

In some embodiments, a subject can be identified as having an increased risk of developing PSP or identified as having PSP (e.g., any of the types of PSP described herein), e.g., at least in part, by detecting tau protein deposits (e.g., 4-repeat tau protein deposits), detecting of atrophy of the midbrain and/or superior cerebellar peduncles (e.g., using any of the imaging techniques described herein or known in the art, e.g., magnetic resonance imaging (MRI) or positron emission tomography (PET) scans), and/or detecting of hypometabolism in the frontal cortex, caudate, and/or thalamus in the subject (e.g., using any of the imaging techniques described herein or known in the art, e.g., MRI, CT scan, or PET scan).

For example, in some embodiments a subject can be identified or diagnosed as having PSP (e.g., any of the types of PSP described herein), e.g., at least in part, by using MRI to detect brain atrophy (Min et al., Nat. Med. 21:1154-1162, 2015; Yanamandra et al., Ann. Clin. Transl. Neurol. 2:278-288, 2015), changes in regional gray and white matter volume to detect atrophy (see, e.g., Josephs et al., Brain 137:2783-2795, 2014; Santos-Santos et al., JAMA Neurol. 73:733-742, 2016), and midbrain atrophy by detecting midbrain area and volume in the subject (Josephs et al., Neurobiol. Aging 29:280-289, 2008, Whitwell et al., Eur. J. Neurol. 20:1417-1422, 2013). In some embodiments, a subject can be identified or diagnosed as having PSP (e.g., any of the types of PSP described herein), e.g., at least in part, by administering to a subject a tau protein tracer (e.g., AV1451 or PBB3) and detecting tau protein in the subject's brain using a PET scan (see, e.g., Marquie et al., Ami. Neurol. 78:787-800, 2015; Cho et al., Mov. Disord. 32:134-140, 2017; Whitwell et al., Mov. Disord. 32:124-133, 2017; and Smith et al., Mov. Disord. 32:108-114, 2017). In some embodiments, a subject can be diagnosed or identified as having PSP (e.g., any of the types of PSP described herein), e.g., at least in part, by detecting the difference in binaural masking level in the subject using a PET scan (see, e.g., Hughes et al., J. Neurophysiol. 112.3086-3094, 2014).

### CSF PSP Biomarkers

In some embodiments, a subject can be identified as being at increased risk of developing PSP or identified as having PSP (e.g., any of the types of PSP described herein), e.g., at least in part, by detecting the presence of, or an elevated level (e.g., as compared to a level in a healthy control subject) of, one or more biomarkers in a subject.

### Tau and Phospho-Tau

Subjects in the methods described herein may have a CSF total tau level that is elevated as compared to a healthy subject. For example, the subject may have a CSF total tau level of about 100 μg/mL or higher. In some embodiments, the subjects have a CSF total tau level of about 300 μg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 μg/mL or higher).

Subjects in the methods described herein may have a CSF phospho-tau (e.g. phospho-tau 181, phospho-tau 199, and/or phosphor-tau 231) that is elevated as compared to a healthy subject. For example, the subject may have a CSF phospho-tau level of about 30 μg/mL or higher. In some embodiments, the subjects have a CSF phospho-Tau (e.g. phosphor-tau 181) level about 70 μg/mL or higher (e.g., about 75, 100, 125, 150, 175, or 200 μg/mL or higher).

### Neurofilament-Light (NfL)

In some embodiments, a subject can be identified or diagnosed as having PSP (e.g., any of the types of PSP described herein), e.g., at least in part, by detecting the presence of, or detection of an elevated level (e.g., as compared to a level in a healthy control subject) of, neurofilament light chain in the blood and/or cerebrospinal fluid in a subject (e.g., using any of the immunoassays described in Scherling et al., Ann. Neurol. 75:116-126, 2014; Bacioglu et al., Neuron 91:56-66, 2016; and Rojas et al., Ann. Clin. Transl. Neurol. 3:216-255, 2016).

Methods of detecting NfL (for example, in the cerebrospinal fluid, plasma, or serum) are known in the art and include but are not limited to, ELISA and Simoa assays (See e.g., Shaw et al. Biochemical and Biophysical Research Communications 336:1268-1277, 2005; Ganesalingam et al. Amyotroph Lateral Scler Frontotemporal Degener 14 (2): 146-9, 2013; De Schaepdryver et al. Annals of Clinical and Translational Neurology 6 (10): 1971-1979, 2019; Wilke et al. Clin Chem Lab Med 57 (10): 1556-1564, 2019; Poesen et al. Front Neurol 9:1167, 2018; Pawlitzki et al. Front. Neurol. 9:1037, 2018; Gille et al. Neuropathol Appl Neurobiol 45 (3): 291-304, 2019). Commercial NfL assay kits based on the Simoa technology, such as those produced by Quanterix can also be used (See, e.g., Thouvenot et al. European Journal of Neurology 27:251-257, 2020). Factors affecting NfL levels or their detection in serum or plasma in relation to disease course may differ from those in CSF. The levels of neurofilament (e.g. pNF-H and/or NfL) in the CSF and serum may be correlated (See, e.g., Wilke et al. Clin Chem Lab Med 57 (10): 1556-1564, 2019).

### YKL-40

In some embodiments, a subject can be identified or diagnosed as having PSP (e.g., any of the types of PSP described herein), e.g., at least in part, by detecting the presence of, or detection of an elevated level (e.g., as compared to a level in a healthy control subject) of, YKL-40 in cerebrospinal fluid from the subject (see, e.g., Magdalinou et al., J. Neurol. Neurosurg. Psychiatry 2014 October; 85 (10): 1065-1075; and Magdalinou et al., J. Neurol. Neurosurg. Psychiatry 86:1240-1247, 2015).

### Ratio of 33 kDa Tau to 55 kDa Tau

In some embodiments, a subject can be identified or diagnosed as having PSP (e.g., any of the types of PSP described herein), e.g., at least in part, by detecting a decreased ratio of 33 kDa tau to SS kDa tau in the CSF of a subject (e.g., as compared to the ratio of 33 kDa tau to 55 kDa tau in a healthy subject).

### Ptpn1

In some embodiments, a subject can be identified or diagnosed as having PSP (e.g., any of the types of PSP described herein), e.g., at least in part, by detecting the presence of, or detecting an elevated level of protein tyrosine phosphatase 1 (Ptpn1) (e.g., as described in Santiago et al., Mov. Discord. 29 (4): 550-555, 2014).

### Neurogranin

In some embodiments, a subject can be identified or diagnosed as having PSP (e.g., any of the types of PSP described herein), e.g., at least in part, by detecting the presence of, or detecting an elevated level of neurogranin (see, e.g., Xiang Y, Xin J, Le W, Yang Y. Neurogranin: A Potential Biomarker of Neurological and Mental Diseases. Front Aging Neurosci. 2020 Oct. 6; 12:584743. doi: 10.3389/fnagi.2020.584743.).

### Other Factors Useful for Diagnosis Subject Selection for PSP

In some embodiments, a subject can be identified or diagnosed as having PSP (e.g., any of the types of PSP described herein), e.g., at least in part, by detecting decreased saccade velocity and gain in the subject using infrared oculography (see, e.g., Boxer et al., Arch. Neurol. 69:509-517, 2012; Boxer et al., Lancet Neurol. 132:676-685, 2014). In some embodiments, a subject can be identified or diagnosed as having PSP (e.g., any of the types of PSP described herein), e.g., at least in part, by detecting a spontaneous and evoked blink rate associated with PSP in the subject (see, e.g., Bologna et al., Brain 132:502-510, 2009). In some embodiments, a subject can be identified or diagnosed as having PSP (e.g., any of the types of PSP described herein), e.g., at least in part, by detecting decreased retinal thickness in a subject's eye using optical coherence tomography (see, e.g., Schneider et al., J. Neural Transm. 121:41-47, 2014). In some embodiments, a subject can be identified or diagnosed as having PSP (e.g., any of the types of PSP described herein), e.g., at least in part, by detecting disrupted circadian rhythms and sleep in the subject (see, e.g., Walsh et al., Sleep Med. 22; 50-56, 2016).

General Selection of Subjects with Various Neurodegenerative Diseases

In some embodiments, the subjects described herein have an “elevated level” of a biomarker (e.g., tau, phospho-tau, NfL, YKL-40, Ptpn1, or neurogranin) in the CSF or blood as compared to a healthy subject who does not have PSP. In some embodiments, an elevated level of a PSP subject can be an elevation or an increase of about 1% to about 500%, about 1% to about 450%, about 1% to about 400%, about 1% to about 350%, about 1% to about 300%, about 1% to about 250%, about 1% to about 200%, about 1% to about 150%, about 1% to about 100%, about 1% to about 50%, about 1% to about 25%, about 1% to about 20%, about 1% to about 15%, about 1% to about 10%, about 1% to about 5%, about 2% to about 500%, about 2% to about 450%, about 2% to about 400%, about 2% to about 350%, about 2% to about 300%, about 2% to about 250%, about 2% to about 200%, about 2% to about 150%, about 2% to about 100%, about 2% to about 50%, about 2% to about 25%, about 2% to about 20%, about 2% to about 15%, about 2% to about 10%, about 5% to about 500%, about 5% to about 450%, about 5% to about 400%, about 5% to about 350%, about 5% to about 300%, about 5% to about 250%, about 5% to about 200%, about 5% to about 150%, about 5% to about 100%, about 5% to about 50%, about 5% to about 25%, about 5% to about 20%, about 5% to about 15%, about 5% to about 10%, about 10% to about 500%, about 10% to about 450%, about 10% to about 400%, about 10% to about 350%, about 10% to about 300%, about 10% to about 250%, about 10% to about 200%, about 10% to about 150%, about 10% to about 100%, about 10% to about 50%, about 10% to about 25%, about 10% to about 20%, about 10% to about 15%, about 15% to about 500%, about 15% to about 450%, about 15% to about 400%, about 15% to about 350%, about 15% to about 300%, about 15% to about 250%, about 15% to about 200%, about 15% to about 150%, about 15% to about 100%, about 15% to about 50%, about 15% to about 25%, about 15% to about 20%, about 20% to about 500%, about 20% to about 450%, about 20% to about 400%, about 20% to about 350%, about 20% to about 300%, about 20% to about 250%, about 20% to about 200%, about 20% to about 150%, about 20% to about 100%, about 20% to about 50%, about 20% to about 25%, about 25% to about 500%, about 25% to about 450%, about 25% to about 400%, about 25% to about 350%, about 25% to about 300%, about 25% to about 250%, about 25% to about 200%, about 25% to about 150%, about 25% to about 100%, about 25% to about 50%, about 50% to about 500%, about 50% to about 450%, about 50% to about 400%, about 50% to about 350%, about 50% to about 300%, about 50% to about 250%, about 50% to about 200%, about 50% to about 150%, about 50% to about 100%, about 100% to about 500%, about 100% to about 450%, about 100% to about 400%, about 100% to about 350%, about 100% to about 300%, about 100% to about 250%, about 100% to about 200%, about 100% to about 150%, about 150% to about 500%, about 150% to about 450%, about 150% to about 400%, about 150% to about 350%, about 150% to about 300%, about 150% to about 250%, about 150% to about 200%, about 200% to about 500%, about 200% to about 450%, about 200% to about 400%, about 200% to about 350%, about 200% to about 300%, about 200% to about 250%, about 250% to about 500%, about 250% to about 450%, about 250% to about 400%, about 250% to about 350%, about 250% to about 300%, about 300% to about 500%, about 300% to about 450%, about 300% to about 400%, about 300% to about 350%, about 350% to about 500%, about 350% to about 450%, about 350% to about 400%, about 400% to about 500%, about 400% to about 450%, or about 450% to about 500%, e.g., as compared to a healthy subject who does not have PSP.

In some embodiments, after administration with any of the compositions described herein (e.g. TURSO and sodium phenylbutyrate), the subjects have a reduction in the level (plasma or CSF) of a biomarker (e.g., tau, phospho-tau, NfL, YKL-40, or Ptpn1). For example, a 1% to about 99% reduction, a 1% to about 95% reduction, a 1% to about 90% reduction, a 1% to about 85% reduction, a 1% to about 80% reduction, a 1% to about 75% reduction, a 1% to about 70% reduction, a 1% to about 65% reduction, a 1% to about 60% reduction, a 1% to about 55% reduction, a 1% to about 50% reduction, a 1% to about 45% reduction, a 1% to about 40% reduction, a 1% to about 35% reduction, a 1% to about 30% reduction, a 1% to about 25% reduction, a 1% to about 20% reduction, a 1% to about 15% reduction, a 1% to about 10% reduction, a 1% to about 5% reduction, an about 5% to about 99% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 99% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 99% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, an about 15% to about 20% reduction, an about 20% to about 99% reduction, an about 20% to about 95% reduction, an about 20% to about 90% reduction, an about 20% to about 85% reduction, an about 20% to about 80% reduction, an about 20% to about 75% reduction, an about 20% to about 70% reduction, an about 20% to about 65% reduction, an about 20% to about 60% reduction, an about 20% to about 55% reduction, an about 20% to about 50% reduction, an about 20% to about 45% reduction, an about 20% to about 40% reduction, an about 20% to about 35% reduction, an about 20% to about 30% reduction, an about 20% to about 25% reduction, an about 25% to about 99% reduction, an about 25% to about 95% reduction, an about 25% to about 90% reduction, an about 25% to about 85% reduction, an about 25% to about 80% reduction, an about 25% to about 75% reduction, an about 25% to about 70% reduction, an about 25% to about 65% reduction, an about 25% to about 60% reduction, an about 25% to about 55% reduction, an about 25% to about 50% reduction, an about 25% to about 45% reduction, an about 25% to about 40% reduction, an about 25% to about 35% reduction, an about 25% to about 30% reduction, an about 30% to about 99% reduction, an about 30% to about 95% reduction, an about 30% to about 90% reduction, an about 30% to about 85% reduction, an about 30% to about 80% reduction, an about 30% to about 75% reduction, an about 30% to about 70% reduction, an about 30% to about 65% reduction, an about 30% to about 60% reduction, an about 30% to about 55% reduction, an about 30% to about 50% reduction, an about 30% to about 45% reduction, an about 30% to about 40% reduction, an about 30% to about 35% reduction, an about 35% to about 99% reduction, an about 35% to about 95% reduction, an about 35% to about 90% reduction, an about 35% to about 85% reduction, an about 35% to about 80% reduction, an about 35% to about 75% reduction, an about 35% to about 70% reduction, an about 35% to about 65% reduction, an about 35% to about 60% reduction, an about 35% to about 55% reduction, an about 35% to about 50% reduction, an about 35% to about 45% reduction, an about 35% to about 40% reduction, an about 40% to about 99% reduction, an about 40% to about 95% reduction, an about 40% to about 90% reduction, an about 40% to about 85% reduction, an about 40% to about 80% reduction, an about 40% to about 75% reduction, an about 40% to about 70% reduction, an about 40% to about 65% reduction, an about 40% to about 60% reduction, an about 40% to about 55% reduction, an about 40% to about 50% reduction, an about 40% to about 45% reduction, an about 45% to about 99% reduction, an about 45% to about 95% reduction, an about 45% to about 90% reduction, an about 45% to about 85% reduction, an about 45% to about 80% reduction, an about 45% to about 75% reduction, an about 45% to about 70% reduction, an about 45% to about 65% reduction, an about 45% to about 60% reduction, an about 45% to about 55% reduction, an about 45% to about 50% reduction, an about 50% to about 99% reduction, an about 50% to about 95% reduction, an about 50% to about 90% reduction, an about 50% to about 85% reduction, an about 50% to about 80% reduction, an about 50% to about 75% reduction, an about 50% to about 70% reduction, an about 50% to about 65% reduction, an about 50% to about 60% reduction, an about 50% to about 55% reduction, an about 55% to about 99% reduction, an about 55% to about 95% reduction, an about 55% to about 90% reduction, an about 55% to about 85% reduction, an about 55% to about 80% reduction, an about 55% to about 75% reduction, an about 55% to about 70% reduction, an about 55% to about 65% reduction, an about 55% to about 60% reduction, an about 60% to about 99% reduction, an about 60% to about 95% reduction, an about 60% to about 90% reduction, an about 60% to about 85% reduction, an about 60% to about 80% reduction, an about 60% to about 75% reduction, an about 60% to about 70% reduction, an about 60% to about 65% reduction, an about 65% to about 99% reduction, an about 65% to about 95% reduction, an about 65% to about 90% reduction, an about 65% to about 85% reduction, an about 65% to about 80% reduction, an about 65% to about 75% reduction, an about 65% to about 70% reduction, an about 70% to about 99% reduction, an about 70% to about 95% reduction, an about 70% to about 90% reduction, an about 70% to about 85% reduction, an about 70% to about 80% reduction, an about 70% to about 75% reduction, an about 75% to about 99% reduction, an about 75% to about 95% reduction, an about 75% to about 90% reduction, an about 75% to about 85% reduction, an about 75% to about 80% reduction, an about 80% to about 99% reduction, an about 80% to about 95% reduction, an about 80% to about 90% reduction, an about 80% to about 85% reduction, an about 85% to about 99% reduction, an about 85% to about 95% reduction, an about 85% to about 90% reduction, an about 90% to about 99% reduction, an about 90% to about 95% reduction, or an about 95% to about 99% reduction, e.g., in a second level (i.e., after treatment with the compositions described herein) of a biomarker (e.g., tau, phospho-tau, NfL, YKL-40, or Ptpn1) as compared to a first level (i.e., prior to treatment with the compositions described herein) of the biomarker (e.g., tau, phospho-tau, NfL, YKL-40, or Ptpn1).

### Bioavailability/Metabolism

Skilled practitioners will appreciate that certain factors can affect the bioavailability and metabolism of the administered compounds for a subject, and can make adjustments accordingly. These include but are not limited to liver function (e.g. levels of liver enzymes), renal function, and gallbladder function (e.g., ion absorption and secretion, levels of cholesterol transport proteins). There can be variability in the levels of exposure each subject has for the administered compounds (e.g., bile acid and a phenylbutyrate compound), differences in the levels of excretion, and in the pharmacokinetics of the compounds in the subjects being treated. Any of the factors described herein may affect drug exposure by the subject. For instance, decreased clearance of the compounds can result in increased drug exposure, while improved renal function can reduce the actual drug exposure. The extent of drug exposure may be correlated with the subject's response to the administered compounds and the outcome of the treatment.

### Subject Age

The subject can be e.g., older than 18 years of age (e.g., between 18-100, 18-90, 18-80, 18-70, 18-60, 18-50, 18-40, 18-30, 18-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 years of age). The subject can have a BMI of between 18.5-30 kg/m² (e.g., between 18.5-28, 18.5-26, 18.5-24, 18.5-22, 18.5-20, 20-30, 20-28, 20-26, 20-24, 20-22, 22-30, 22-28, 22-26, 22-24, 24-30, 24-28, 24-26, 26-30, 26-28, or 28-30 kg/m²).

### Methods of Treatment

The present disclosure provides methods of treating a neurodegenerative disease (e.g., AD or PSP) in a subject, or ameliorating at least one symptom of a neurodegenerative disease (e.g., AD or PSP) in a subject, or prophylactically treating a subject at risk for developing a neurodegenerative disease (e.g., AD or PSP) (e.g., a subject who carries one or more copies of the ApoEε4 allele) or a subject suspected to be developing a neurodegenerative disease (e.g., a subject displaying at least one symptom of AD, or at least one symptom of PSP).

Some embodiments of the present disclosure provide methods of slowing a neurodegenerative disease (e.g., AD or PSP) disease progression (e.g., reducing the AD or PSPdisease progression rate); and methods of reducing and/or preventing progressive decline of cognitive functions, including loss of declarative and procedural memory, decreased learning ability, reduced attention span, and severe impairment in thinking ability, judgment, and decision making.

Also provided herein are methods of ameliorating one or more biomarkers that are affected in a neurodegenerative disease (e.g., AD or PSP) patients. For example, in some instances, provided herein are methods to reduce total tau and/or phospho-tau in the CSF, serum, or blood, etc.

Generally, also provided in the present disclosure are methods of treating a tauopathy in a subject, or ameliorating at least one symptom of a tauopathy in a subject, or prophylactically treating a subject at risk for developing a tauopathy or a subject suspected to be developing a tauopathy. Some embodiments of the present disclosure provide methods of slowing a tauopathy disease progression; and methods of reducing and/or preventing progressive decline of various functions associated with the tauopathy (e.g., in some instances this may be cognitive functions). Also provided herein are methods of ameliorating one or more biomarkers that are affected in a tauopathy patients

In some embodiments of any of the methods described herein, the methods include administering to the subject a bile acid or pharmaceutically acceptable salt thereof, and a phenylbutyrate compound. In some embodiments, the methods described herein include administering to a subject about 10 mg/kg to about 50 mg/kg (e.g., about 10 mg/kg to about 48 mg/kg, about 10 mg/kg to about 46 mg/kg, about 10 mg/kg to about 44 mg/kg, about 10 mg/kg to about 42 mg/kg, about 10 mg/kg to about 40 mg/kg, about 10 mg/kg to about 38 mg/kg, about 10 mg/kg to about 36 mg/kg, about 10 mg/kg to about 34 mg/kg, about 10 mg/kg to about 32 mg/kg, about 10 mg/kg to about 30 mg/kg, about 10 mg/kg to about 28 mg/kg, about 10 mg/kg to about 26 mg/kg, about 10 mg/kg to about 24 mg/kg, about 10 mg/kg to about 22 mg/kg, about 10 mg/kg to about 20 mg/kg, about 10 mg/kg to about 18 mg/kg, about 10 mg/kg to about 16 mg/kg, about 10 mg/kg to about 14 mg/kg, about 10 mg/kg to about 12 mg/kg, about 12 mg/kg to about 50 mg/kg, about 12 mg/kg to about 48 mg/kg, about 12 mg/kg to about 46 mg/kg, about 12 mg/kg to about 44 mg/kg, about 12 mg/kg to about 42 mg/kg, about 12 mg/kg to about 40 mg/kg, about 12 mg/kg to about 38 mg/kg, about 12 mg/kg to about 36 mg/kg, about 12 mg/kg to about 34 mg/kg, about 12 mg/kg to about 32 mg/kg, about 12 mg/kg to about 30 mg/kg, about 12 mg/kg to about 28 mg/kg, about 12 mg/kg to about 26 mg/kg, about 12 mg/kg to about 24 mg/kg, about 12 mg/kg to about 22 mg/kg, about 12 mg/kg to about 20 mg/kg, about 12 mg/kg to about 18 mg/kg, about 12 mg/kg to about 16 mg/kg, about 12 mg/kg to about 14 mg/kg, about 14 mg/kg to about 50 mg/kg, about 14 mg/kg to about 48 mg/kg, about 14 mg/kg to about 46 mg/kg, about 14 mg/kg to about 44 mg/kg, about 14 mg/kg to about 42 mg/kg, about 14 mg/kg to about 40 mg/kg, about 14 mg/kg to about 38 mg/kg, about 14 mg/kg to about 36 mg/kg, about 14 mg/kg to about 34 mg/kg, about 14 mg/kg to about 32 mg/kg, about 14 mg/kg to about 30 mg/kg, about 14 mg/kg to about 28 mg/kg, about 14 mg/kg to about 26 mg/kg, about 14 mg/kg to about 24 mg/kg, about 14 mg/kg to about 22 mg/kg, about 14 mg/kg to about 20 mg/kg, about 14 mg/kg to about 18 mg/kg, about 14 mg/kg to about 16 mg/kg, about 16 mg/kg to about 50 mg/kg, about 16 mg/kg to about 48 mg/kg, about 16 mg/kg to about 46 mg/kg, about 16 mg/kg to about 44 mg/kg, about 16 mg/kg to about 42 mg/kg, about 16 mg/kg to about 40 mg/kg, about 16 mg/kg to about 38 mg/kg, about 16 mg/kg to about 36 mg/kg, about 16 mg/kg to about 34 mg/kg, about 16 mg/kg to about 32 mg/kg, about 16 mg/kg to about 30 mg/kg, about 16 mg/kg to about 28 mg/kg, about 16 mg/kg to about 26 mg/kg, about 16 mg/kg to about 24 mg/kg, about 16 mg/kg to about 22 mg/kg, about 16 mg/kg to about 20 mg/kg, about 16 mg/kg to about 18 mg/kg, about 18 mg/kg to about 50 mg/kg, about 18 mg/kg to about 48 mg/kg, about 18 mg/kg to about 46 mg/kg, about 18 mg/kg to about 44 mg/kg, about 18 mg/kg to about 42 mg/kg, about 18 mg/kg to about 40 mg/kg, about 18 mg/kg to about 38 mg/kg, about 18 mg/kg to about 36 mg/kg, about 18 mg/kg to about 34 mg/kg, about 18 mg/kg to about 32 mg/kg, about 18 mg/kg to about 30 mg/kg, about 18 mg/kg to about 28 mg/kg, about 18 mg/kg to about 26 mg/kg, about 18 mg/kg to about 24 mg/kg, about 18 mg/kg to about 22 mg/kg, about 18 mg/kg to about 20 mg/kg, about 20 mg/kg to about 50 mg/kg, about 20 mg/kg to about 48 mg/kg, about 20 mg/kg to about 46 mg/kg, about 20 mg/kg to about 44 mg/kg, about 20 mg/kg to about 42 mg/kg, about 20 mg/kg to about 40 mg/kg, about 20 mg/kg to about 38 mg/kg, about 20 mg/kg to about 36 mg/kg, about 20 mg/kg to about 34 mg/kg, about 20 mg/kg to about 32 mg/kg, about 20 mg/kg to about 30 mg/kg, about 20 mg/kg to about 28 mg/kg, about 20 mg/kg to about 26 mg/kg, about 20 mg/kg to about 24 mg/kg, about 20 mg/kg to about 22 mg/kg, about 22 mg/kg to about 50 mg/kg, about 22 mg/kg to about 48 mg/kg, about 22 mg/kg to about 46 mg/kg, about 22 mg/kg to about 44 mg/kg, about 22 mg/kg to about 42 mg/kg, about 22 mg/kg to about 40 mg/kg, about 22 mg/kg to about 38 mg/kg, about 22 mg/kg to about 36 mg/kg, about 22 mg/kg to about 34 mg/kg, about 22 mg/kg to about 32 mg/kg, about 22 mg/kg to about 30 mg/kg, about 22 mg/kg to about 28 mg/kg, about 22 mg/kg to about 26 mg/kg, about 22 mg/kg to about 24 mg/kg, about 24 mg/kg to about 50 mg/kg, about 24 mg/kg to about 48 mg/kg, about 24 mg/kg to about 46 mg/kg, about 24 mg/kg to about 44 mg/kg, about 24 mg/kg to about 42 mg/kg, about 24 mg/kg to about 40 mg/kg, about 24 mg/kg to about 38 mg/kg, about 24 mg/kg to about 36 mg/kg, about 24 mg/kg to about 34 mg/kg, about 24 mg/kg to about 32 mg/kg, about 24 mg/kg to about 30 mg/kg, about 24 mg/kg to about 28 mg/kg, about 24 mg/kg to about 26 mg/kg, about 26 mg/kg to about 50 mg/kg, about 26 mg/kg to about 48 mg/kg, about 26 mg/kg to about 46 mg/kg, about 26 mg/kg to about 44 mg/kg, about 26 mg/kg to about 42 mg/kg, about 26 mg/kg to about 40 mg/kg, about 26 mg/kg to about 38 mg/kg, about 26 mg/kg to about 36 mg/kg, about 26 mg/kg to about 34 mg/kg, about 26 mg/kg to about 32 mg/kg, about 26 mg/kg to about 30 mg/kg, about 26 mg/kg to about 28 mg/kg, about 28 mg/kg to about 50 mg/kg, about 28 mg/kg to about 48 mg/kg, about 28 mg/kg to about 46 mg/kg, about 28 mg/kg to about 44 mg/kg, about 28 mg/kg to about 42 mg/kg, about 28 mg/kg to about 40 mg/kg, about 28 mg/kg to about 38 mg/kg, about 28 mg/kg to about 36 mg/kg, about 28 mg/kg to about 34 mg/kg, about 28 mg/kg to about 32 mg/kg, about 28 mg/kg to about 30 mg/kg, about 30 mg/kg to about 50 mg/kg, about 30 mg/kg to about 48 mg/kg, about 30 mg/kg to about 46 mg/kg, about 30 mg/kg to about 44 mg/kg, about 30 mg/kg to about 42 mg/kg, about 30 mg/kg to about 40 mg/kg, about 30 mg/kg to about 38 mg/kg, about 30 mg/kg to about 36 mg/kg, about 30 mg/kg to about 34 mg/kg, about 30 mg/kg to about 32 mg/kg, about 32 mg/kg to about 50 mg/kg, about 32 mg/kg to about 48 mg/kg, about 32 mg/kg to about 46 mg/kg, about 32 mg/kg to about 44 mg/kg, about 32 mg/kg to about 42 mg/kg, about 32 mg/kg to about 40 mg/kg, about 32 mg/kg to about 38 mg/kg, about 32 mg/kg to about 36 mg/kg, about 32 mg/kg to about 34 mg/kg, about 34 mg/kg to about 50 mg/kg, about 34 mg/kg to about 48 mg/kg, about 34 mg/kg to about 46 mg/kg, about 34 mg/kg to about 44 mg/kg, about 34 mg/kg to about 42 mg/kg, about 34 mg/kg to about 40 mg/kg, about 34 mg/kg to about 38 mg/kg, about 34 mg/kg to about 36 mg/kg, about 36 mg/kg to about 50 mg/kg, about 36 mg/kg to about 48 mg/kg, about 36 mg/kg to about 46 mg/kg, about 36 mg/kg to about 44 mg/kg, about 36 mg/kg to about 42 mg/kg, about 36 mg/kg to about 40 mg/kg, about 36 mg/kg to about 38 mg/kg, about 38 mg/kg to about 50 mg/kg, about 38 mg/kg to about 48 mg/kg, about 38 mg/kg to about 46 mg/kg, about 38 mg/kg to about 44 mg/kg, about 38 mg/kg to about 42 mg/kg, about 38 mg/kg to about 40 mg/kg, about 40 mg/kg to about 50 mg/kg, about 40 mg/kg to about 48 mg/kg, about 40 mg/kg to about 46 mg/kg, about 40 mg/kg to about 44 mg/kg, about 40 mg/kg to about 42 mg/kg, about 42 mg/kg to about 50 mg/kg, about 42 mg/kg to about 48 mg/kg, about 42 mg/kg to about 46 mg/kg, about 42 mg/kg to about 44 mg/kg, about 44 mg/kg to about 50 mg/kg, about 44 mg/kg to about 48 mg/kg, about 44 mg/kg to about 46 mg/kg, about 46 mg/kg to about 50 mg/kg, about 46 mg/kg to about 48 mg/kg, or about 46 mg/kg to about 50 mg/kg) of body weight of a bile acid (e.g., any of the bile acids described herein or known in the art e.g., TURSO) or a pharmaceutically acceptable salt thereof, and about 10 mg/kg to about 400 mg/kg (e.g., about 10 mg/kg to about 380 mg/kg, about 10 mg/kg to about 360 mg/kg, about 10 mg/kg to about 340 mg/kg, about 10 mg/kg to about 320 mg/kg, about 10 mg/kg to about 300 mg/kg, about 10 mg/kg to about 280 mg/kg, about 10 mg/kg to about 260 mg/kg, about 10 mg/kg to about 240 mg/kg, about 10 mg/kg to about 220 mg/kg, about 10 mg/kg to about 200 mg/kg, about 10 mg/kg to about 180 mg/kg, about 10 mg/kg to about 160 mg/kg, about 10 mg/kg to about 140 mg/kg, about 10 mg/kg to about 120 mg/kg, about 10 mg/kg to about 100 mg/kg, about 10 mg/kg to about 80 mg/kg, about 10 mg/kg to about 60 mg/kg, about 10 mg/kg to about 40 mg/kg, about 10 mg/kg to about 20 mg/kg, about 20 mg/kg to about 400 mg/kg, about 20 mg/kg to about 380 mg/kg, about 20 mg/kg to about 360 mg/kg, about 20 mg/kg to about 340 mg/kg, about 20 mg/kg to about 320 mg/kg, about 20 mg/kg to about 300 mg/kg, about 20 mg/kg to about 280 mg/kg, about 20 mg/kg to about 260 mg/kg, about 20 mg/kg to about 240 mg/kg, about 20 mg/kg to about 220 mg/kg, about 20 mg/kg to about 200 mg/kg, about 20 mg/kg to about 180 mg/kg, about 20 mg/kg to about 160 mg/kg, about 20 mg/kg to about 140 mg/kg, about 20 mg/kg to about 120 mg/kg, about 20 mg/kg to about 100 mg/kg, about 20 mg/kg to about 80 mg/kg, about 20 mg/kg to about 60 mg/kg, about 20 mg/kg to about 40 mg/kg, about 40 mg/kg to about 400 mg/kg, about 40 mg/kg to about 380 mg/kg, about 40 mg/kg to about 360 mg/kg, about 40 mg/kg to about 340 mg/kg, about 40 mg/kg to about 320 mg/kg, about 40 mg/kg to about 300 mg/kg, about 40 mg/kg to about 280 mg/kg, about 40 mg/kg to about 260 mg/kg, about 40 mg/kg to about 240 mg/kg, about 40 mg/kg to about 220 mg/kg, about 40 mg/kg to about 200 mg/kg, about 40 mg/kg to about 180 mg/kg, about 40 mg/kg to about 160 mg/kg, about 40 mg/kg to about 140 mg/kg, about 40 mg/kg to about 120 mg/kg, about 40 mg/kg to about 100 mg/kg, about 40 mg/kg to about 80 mg/kg, about 40 mg/kg to about 60 mg/kg, about 60 mg/kg to about 400 mg/kg, about 60 mg/kg to about 380 mg/kg, about 60 mg/kg to about 360 mg/kg, about 60 mg/kg to about 340 mg/kg, about 60 mg/kg to about 320 mg/kg, about 60 mg/kg to about 300 mg/kg, about 60 mg/kg to about 280 mg/kg, about 60 mg/kg to about 260 mg/kg, about 60 mg/kg to about 240 mg/kg, about 60 mg/kg to about 220 mg/kg, about 60 mg/kg to about 200 mg/kg, about 60 mg/kg to about 180 mg/kg, about 60 mg/kg to about 160 mg/kg, about 60 mg/kg to about 140 mg/kg, about 60 mg/kg to about 120 mg/kg, about 60 mg/kg to about 100 mg/kg, about 60 mg/kg to about 80 mg/kg, about 80 mg/kg to about 400 mg/kg, about 80 mg/kg to about 380 mg/kg, about 80 mg/kg to about 360 mg/kg, about 80 mg/kg to about 340 mg/kg, about 80 mg/kg to about 320 mg/kg, about 80 mg/kg to about 300 mg/kg, about 80 mg/kg to about 280 mg/kg, about 80 mg/kg to about 260 mg/kg, about 80 mg/kg to about 240 mg/kg, about 80 mg/kg to about 220 mg/kg, about 80 mg/kg to about 200 mg/kg, about 80 mg/kg to about 180 mg/kg, about 80 mg/kg to about 160 mg/kg, about 80 mg/kg to about 140 mg/kg, about 80 mg/kg to about 120 mg/kg, about 80 mg/kg to about 100 mg/kg, about 100 mg/kg to about 400 mg/kg, about 100 mg/kg to about 380 mg/kg, about 100 mg/kg to about 360 mg/kg, about 100 mg/kg to about 340 mg/kg, about 100 mg/kg to about 320 mg/kg, about 100 mg/kg to about 300 mg/kg, about 100 mg/kg to about 280 mg/kg, about 100 mg/kg to about 260 mg/kg, about 100 mg/kg to about 240 mg/kg, about 100 mg/kg to about 220 mg/kg, about 100 mg/kg to about 200 mg/kg, about 100 mg/kg to about 180 mg/kg, about 100 mg/kg to about 160 mg/kg, about 100 mg/kg to about 140 mg/kg, about 100 mg/kg to about 120 mg/kg, about 120 mg/kg to about 400 mg/kg, about 120 mg/kg to about 380 mg/kg, about 120 mg/kg to about 360 mg/kg, about 120 mg/kg to about 340 mg/kg, about 120 mg/kg to about 320 mg/kg, about 120 mg/kg to about 300 mg/kg, about 120 mg/kg to about 280 mg/kg, about 120 mg/kg to about 260 mg/kg, about 120 mg/kg to about 240 mg/kg, about 120 mg/kg to about 220 mg/kg, about 120 mg/kg to about 200 mg/kg, about 120 mg/kg to about 180 mg/kg, about 120 mg/kg to about 160 mg/kg, about 120 mg/kg to about 140 mg/kg, about 140 mg/kg to about 400 mg/kg, about 140 mg/kg to about 380 mg/kg, about 140 mg/kg to about 360 mg/kg, about 140 mg/kg to about 340 mg/kg, about 140 mg/kg to about 320 mg/kg, about 140 mg/kg to about 300 mg/kg, about 140 mg/kg to about 280 mg/kg, about 140 mg/kg to about 260 mg/kg, about 140 mg/kg to about 240 mg/kg, about 140 mg/kg to about 220 mg/kg, about 140 mg/kg to about 200 mg/kg, about 140 mg/kg to about 180 mg/kg, about 140 mg/kg to about 160 mg/kg, about 160 mg/kg to about 400 mg/kg, about 160 mg/kg to about 380 mg/kg, about 160 mg/kg to about 360 mg/kg, about 160 mg/kg to about 340 mg/kg, about 160 mg/kg to about 320 mg/kg, about 160 mg/kg to about 300 mg/kg, about 160 mg/kg to about 280 mg/kg, about 160 mg/kg to about 260 mg/kg, about 160 mg/kg to about 240 mg/kg, about 160 mg/kg to about 220 mg/kg, about 160 mg/kg to about 200 mg/kg, about 160 mg/kg to about 180 mg/kg, about 180 mg/kg to about 400 mg/kg, about 180 mg/kg to about 380 mg/kg, about 180 mg/kg to about 360 mg/kg, about 180 mg/kg to about 340 mg/kg, about 180 mg/kg to about 320 mg/kg, about 180 mg/kg to about 300 mg/kg, about 180 mg/kg to about 280 mg/kg, about 180 mg/kg to about 260 mg/kg, about 180 mg/kg to about 240 mg/kg, about 180 mg/kg to about 220 mg/kg, about 180 mg/kg to about 200 mg/kg, about 200 mg/kg to about 400 mg/kg, about 200 mg/kg to about 380 mg/kg, about 200 mg/kg to about 360 mg/kg, about 200 mg/kg to about 340 mg/kg, about 200 mg/kg to about 320 mg/kg, about 200 mg/kg to about 300 mg/kg, about 200 mg/kg to about 280 mg/kg, about 200 mg/kg to about 260 mg/kg, about 200 mg/kg to about 240 mg/kg, about 200 mg/kg to about 220 mg/kg, about 220 mg/kg to about 400 mg/kg, about 220 mg/kg to about 380 mg/kg, about 220 mg/kg to about 360 mg/kg, about 220 mg/kg to about 340 mg/kg, about 220 mg/kg to about 320 mg/kg, about 220 mg/kg to about 300 mg/kg, about 220 mg/kg to about 280 mg/kg, about 220 mg/kg to about 260 mg/kg, about 220 mg/kg to about 240 mg/kg, about 240 mg/kg to about 400 mg/kg, about 240 mg/kg to about 380 mg/kg, about 240 mg/kg to about 360 mg/kg, about 240 mg/kg to about 340 mg/kg, about 240 mg/kg to about 320 mg/kg, about 240 mg/kg to about 300 mg/kg, about 240 mg/kg to about 280 mg/kg, about 240 mg/kg to about 260 mg/kg, about 260 mg/kg to about 400 mg/kg, about 260 mg/kg to about 380 mg/kg, about 260 mg/kg to about 360 mg/kg, about 260 mg/kg to about 340 mg/kg, about 260 mg/kg to about 320 mg/kg, about 260 mg/kg to about 300 mg/kg, about 260 mg/kg to about 280 mg/kg, about 280 mg/kg to about 400 mg/kg, about 280 mg/kg to about 380 mg/kg, about 280 mg/kg to about 360 mg/kg, about 280 mg/kg to about 340 mg/kg, about 280 mg/kg to about 320 mg/kg, about 280 mg/kg to about 300 mg/kg, about 300 mg/kg to about 400 mg/kg, about 300 mg/kg to about 380 mg/kg, about 300 mg/kg to about 360 mg/kg, about 300 mg/kg to about 340 mg/kg, about 300 mg/kg to about 320 mg/kg, about 320 mg/kg to about 400 mg/kg, about 320 mg/kg to about 380 mg/kg, about 320 mg/kg to about 360 mg/kg, about 320 mg/kg to about 340 mg/kg, about 340 mg/kg to about 400 mg/kg, about 340 mg/kg to about 380 mg/kg, about 340 mg/kg to about 360 mg/kg, about 360 mg/kg to about 400 mg/kg, about 360 mg/kg to about 380 mg/kg, or about 380 mg/kg to about 400 mg/kg) of body weight of a phenylbutyrate compound (e.g., any of the phenylbutyrate compounds described herein or known in the art, e.g., sodium phenylbutyrate).

In some embodiments, the bile acid (e.g., TURSO) is administered in an amount of about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, or about 70 mg/kg of body weight. In some embodiments, the phenylbutyrate compound (e.g., sodium phenylbutyrate) is administered in an amount of about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, about 300 mg/kg, about 320 mg/kg, about 340 mg/kg, about 360 mg/kg, about 380 mg/kg, or about 400 mg/kg of body weight.

The bile acid or a pharmaceutically acceptable salt thereof and the phenylbutyrate compound can be administered separately or concurrently, including as a part of a regimen of treatment. The compounds can be administered daily, weekly, monthly, or quarterly. In some embodiments, the compounds are administered once a day, twice a day, or three times a day or more. The compounds can be administered over a period of weeks, months, or years. For example, the compounds can be administered over a period of at least or about 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, or at least or about 5 years, or more. The bile acid and phenylbutyrate compound can, for example, be administered once a day or twice a day for 60 days or less (e.g., 55 days, 50 days, 45 days, 40 days, 35 days, 30 days or less). Alternatively, the bile acid and phenylbutyrate compounds can be administered once a day or twice a day for more than 60 days (e.g., more than 65, 70, 75, 80, 85, 90, 95, 100, 105, 110, 115, 120, 130, 140, 150, 160, 180, 200, 250, 300, 400, 500, 600 days).

In some embodiments of any of the methods described herein, the bile acid is TURSO. TURSO can be administered to a subject at a dose of about 0.5 grams to about 10 grams daily (e.g., about 1, 2, 3, 4, 5, 6, 7, 8, or 9 grams daily). For example, TURSO can be administered at an amount of about 0.5 to about 5 grams (e.g., about 0.5 to about 4.5, about 0.5 to about 4, about 0.5 to about 3.5, about 0.5 to about 3, about 0.5 to about 2.5, about 0.5 to about 2, about 0.5 to about 1.5, about 0.5 to about 1, about 1 to about 5, about 1 to about 4.5, about 1 to about 4, about 1 to about 3.5, about 1 to about 3, about 1 to about 2.5, about 1 to about 2, about 1 to about 1.5, about 1.5 to about 5, about 1.5 to about 4.5, about 1.5 to about 4, about 1.5 to about 3.5, about 1.5 to about 3, about 1.5 to about 2.5, about 1.5 to about 2, about 2 to about 5, about 2 to about 4.5, about 2 to about 4, about 2 to about 3.5, about 2 to about 3, about 2 to about 2.5, about 2.5 to about 5, about 2.5 to about 4.5, about 2.5 to about 4, about 2.5 to about 3.5, about 2.5 to about 3, about 3 to about 5, about 3 to about 4.5, about 3 to about 4, about 3 to about 3.5, about 3.5 to about 5 about 3.5 to about 4.5, about 3.5 to about 4, about 4 to about 5, about 4 to about 4.5, or about 4.5 to about 5 grams) per day. In some embodiments, TURSO is administered to a subject at an amount of about 1 gram per day. In some embodiments, TURSO is administered to a subject at an amount of about 2 grams per day. For example, TURSO can be administered at an amount of about 1 gram twice a day.

In some embodiments of any of the methods described herein, the phenylbutyrate compound is sodium phenylbutyrate. Sodium phenylbutyrate can be administered at an amount of about 1 gram to about 30 grams daily (e.g., about 2, 3, 4, 5, 6, 7, 8, 9, 10, 11, 12, 13, 14, 15, 16, 17, 18, 19, 20, 21, 22, 23, 24, 25, 26, 27, 28, or 29 grams daily). For example, sodium phenylbutyrate can be administered at an amount of about 0.5 to about 10 grams (e.g., about 0.5 to about 9.5, about 0.5 to about 9, about 0.5 to about 8.5, about 0.5 to about 8, about 0.5 to about 7.5, about 0.5 to about 7, about 0.5 to about 6.5, about 0.5 to about 6, about 0.5 to about 5 S, about 0.5 to about 5, about 0.5 to about 4.5, about 0.5 to about 4, about 0.5 to about 3.5, about 0.5 to about 3, about 0.5 to about 2.5, about 0.5 to about 2, about 0.5 to about 1.5, about 0.5 to about 1, about 1 to about 10, about 1 to about 9.5, about 1 to about 9, about 1 to about 8.5, about 1 to about 8, about 1 to about 7.5, about 1 to about 7, about 1 to about 6.5, about 1 to about 6, about 1 to about 5.5, about 1 to about 5, about 1 to about 4.5, about 1 to about 4, about 1 to about 3.5, about 1 to about 3, about 1 to about 2.5, about 1 to about 2, about 1 to about 1.5, about 1.5 to about 10, about 1.5 to about 9.5, about 1.5 to about 9, about 1.5 to about 8.5, about 1.5 to about 8, about 1.5 to about 7.5, about 1.5 to about 7, about 1.5 to about 6.5, about 1.5 to about 6, about 1.5 to about 5.5, about 1.5 to about 5, about 1.5 to about 4.5, about 1.5 to about 4, about 1.5 to about 3.5, about 1.5 to about 3, about 1.5 to about 2.5, about 1.5 to about 2, about 2 to about 10, about 2 to about 9.5, about 2 to about 9, about 2 to about 8.5, about 2 to about 8, about 2 to about 7.5, about 2 to about 7, about 2 to about 6.5, about 2 to about 6, about 2 to about 5.5, about 2 to about 5, about 2 to about 4.5, about 2 to about 4, about 2 to about 3.5, about 2 to about 3, about 2 to about 2.5, about 2.5 to about 10, about 2.5 to about 9.5, about 2.5 to about 9, about 2.5 to about 8.5, about 2.5 to about 8, about 2.5 to about 7.5, about 2.5 to about 7, about 2.5 to about 6.5, about 2.5 to about 6, about 2.5 to about 5.5, about 2.5 to about 5, about 2.5 to about 4.5, about 2.5 to about 4, about 2.5 to about 3.5, about 2.5 to about 3, about 3 to about 10, about 3 to about 9.5, about 3 to about 9, about 3 to about 8.5, about 3 to about 8, about 3 to about 7.5, about 3 to about 7, about 3 to about 6.5, about 3 to about 6, about 3 to about 5.5, about 3 to about 5, about 3 to about 4.5, about 3 to about 4, about 3 to about 3.5, about 3.5 to about 10, about 3.5 to about 9.5, about 3.5 to about 9, about 3.5 to about 8.5, about 3.5 to about 8, about 3.5 to about 7.5, about 3.5 to about 7, about 3.5 to about 6.5, about 3.5 to about 6, about 3.5 to about 5.5, about 3.5 to about 5, about 3.5 to about 4.5, about 3.5 to about 4, about 4 to about 10, about 4 to about 9.5, about 4 to about 9, about 4 to about 8.5, about 4 to about 8, about 4 to about 7.5, about 4 to about 7, about 4 to about 6.5, about 4 to about 6, about 4 to about 5.5, about 4 to about 5, about 4 to about 4.5, about 4.5 to about 10, about 4.5 to about 9.5, about 4.5 to about 9, about 4.5 to about 8.5, about 4.5 to about 8, about 4.5 to about 7.5, about 4.5 to about 7, about 4.5 to about 6.5, about 4.5 to about 6, about 4.5 to about 5.5, about 4.5 to about 5, about 5 to about 10, about 5 to about 9.5, about 5 to about 9, about 5 to about 8.5, about 5 to about 8, about 5 to about 7.5, about 5 to about 7, about 5 to about 6.5, about 5 to about 6, about 5 to about 5.5, about 5.5 to about 10, about 5.5 to about 9.5, about 5.5 to about 9, about 5.5 to about 8.5, about 5.5 to about 8, about 5.5 to about 7.5, about 5.5 to about 7, about 5.5 to about 6.5, about 5.5 to about 6, about 6 to about 10, about 6 to about 9.5, about 6 to about 9, about 6 to about 8.5, about 6 to about 8, about 6 to about 7.5, about 6 to about 7, about 6 to about 6.5, about 6.5 to about 10, about 6.5 to about 9.5, about 6.5 to about 9, about 6.5 to about 8.5, about 6.5 to about 8, about 6.5 to about 7.5, about 6.5 to about 7, about 7 to about 10, about 7 to about 9.5, about 7 to about 9, about 7 to about 8.5, about 7 to about 8, about 7 to about 7.5, about 7.5 to about 10, about 7.5 to about 9.5, about 7.5 to about 9, about 7.5 to about 8.5, about 7.5 to about 8, about 8 to about 10, about 8 to about 9.5, about 8 to about 9, about 8 to about 8.5, about 8.5 to about 10, about 8.5 to about 9.5, about 8.5 to about 9, about 9 to about 10, about 9 to about 9.5, or about 9.5 to about 10 grams) per day. In some embodiments, sodium phenylbutyrate is administered at an amount of about 3 grams per day. In some embodiments, sodium phenylbutyrate is administered at an amount of about 6 grams per day. For example, sodium phenylbutyrate can be administered at an amount of about 3 grams twice a day. In some embodiments, the bile acid and phenylbutyrate compound are administered at a ratio by weight of about 2.5:1 to about 3.5:1 (e.g., about 3:1).

In some embodiments of any of the methods described herein, the methods include administering TURSO and sodium phenylbutyrate to the subject according to a first regimen followed by a second regimen, where the first regimen includes administering about 1 gram of TURSO once a day and about 3 grams of sodium phenylbutyrate once a day for at least 14 days (e.g., at least 16, 18, 21, 24, 27, 30, 35, or 40 days), and the second regimen includes administering about 1 gram of TURSO twice a day and about 3 grams of sodium phenylbutyrate twice a day for at least 30 days (e.g., at least 35, 40, 45, 50, 60, 80, 100, 120, 150, 180, 250, 300, or 400 days).

In some embodiments of any of the methods described herein, the subject is diagnosed with AD, at risk for developing AD, or suspected as having AD. The subject may, for example, have been diagnosed with AD for 24 months or less (e.g., any of the subranges within this range described herein). For example, the subject may have been diagnosed with AD for 1 week or less, or on the same day that the presently disclosed treatments are administered. The subject may have shown one or more symptoms of AD for 24 months or less (e.g., any of the subranges within this range described herein), has elevated levels of total tau, phospho-tau, neurofilament-light (NfL), Ubiquitin carboxyl-terminal hydrolase L1 (UCHL1)/PGP9.5, Glial fibrillary acidic protein (GFAP), 8-hydoxy-2′-deoxyguanosine (8-OHdG), Soluble insulin receptor (sIR); has reduced CSF Aβ₁₋₄₂ levels; have a mutation in one or more genes selected from the group consisting of: APOE (e.g. carrying one or more copies of the APOEε4 allele), APP, PSEN1, and PSEN2.

In some embodiments of any of the methods described herein, the subject is diagnosed with a neurodegenerative disease (e.g., a tauopathy like PSP), at risk for developing a neurodegenerative disease (e.g., a tauopathy like PSP), or suspected as having a neurodegenerative disease (e.g., a tauopathy like PSP). The subject may, for example, have been diagnosed with a neurodegenerative disease (e.g., a tauopathy like PSP) for 24 months or less (e.g., any of the subranges within this range described herein). For example, the subject may have been diagnosed with a neurodegenerative disease (e.g., a tauopathy like PSP) for 1 week or less, or on the same day that the presently disclosed treatments are administered. The subject may have shown one or more symptoms of a neurodegenerative disease (e.g., a tauopathy like PSP) for 24 months or less (e.g., any of the subranges within this range described herein), has elevated levels of total tau, phospho-tau, neurofilament-light (NfL), or YKL-40.

In some embodiments, prior to treatment the subjects have a baseline CSF total tau level of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher). In some embodiments, administration of the bile acid (e.g. TURSO) and the phenylbutyrate compound (e.g. sodium phenylbutyrate) reduces the levels of CSF total tau by about 35 pg/mL or more (e.g., about 40, 45, 50, 55, 60, 65, 70, 75, 80, 85, 90, 95 pg/mL or more).

In some embodiments, prior to treatment the subjects have a baseline CSF phospho-tau (e.g. phospho-tau 181, phospho-tau 199, and/or phospho-tau 231) level of about 30 pg/mL or higher. In some embodiments, prior to treatment the subjects have a baseline CSF phospho-Tau (e.g. phospho-tau 181) level of about 70 pg/mL or higher (e.g., about 75, 100, 125, 150, 175, or 200 pg/mL or higher). In some embodiments, administration of the bile acid (e.g. TURSO) and the phenylbutyrate compound (e.g. sodium phenylbutyrate) reduces the levels of CSF phospho-tau by about 5 pg/mL or more (e.g., about 10, 15, 20, 25, 30, 35, 40, 45, 50, 55, 60 pg/mL or more). In some embodiments, prior to treatment the subjects have a baseline CSF Fatty acid-binding protein 3 (FABP3) level of about 2000 pg/mL or higher (e.g., about 2200, 2500, 2800, 3200, 3500, 3800, 3900, 4000, 4100, or 4200 pg/mL or higher). In some embodiments, administration of the bile acid (e.g. TURSO) and the phenylbutyrate compound (e.g. sodium phenylbutyrate) reduces the levels of CSF FABP3 by about 200 pg/mL or more (e.g., about 250, 280, 310, 320, 330, 340, 350, 360, 370, 380, 390, 400, 410, 420, 430, 440, 450, 460, 470, 480, 490, 500 pg/mL or more).

In some embodiments, prior to treatment the subjects have a baseline CSF neurogranin level of 200 pg/mL or higher (e.g., about 250, 300, 350, 400, 450, 500, 550, 600, 650, 700, 750, 800, 850, 900, 950, 1000 pg/mL or higher). In some embodiments, administration of the bile acid (e.g. TURSO) and the phenylbutyrate compound (e.g. sodium phenylbutyrate) reduces the levels of CSF neurogranin by about 30 pg/mL or more (e.g., about 35, 40, 45, 50, 55, 60, 70, 80, 90, 100, 200 pg/mL or more).

In some embodiments, prior to treatment the subjects have a baseline CSF YKL-40 level of 140000 pg/mL or higher (e.g., about 160000, 180000, 210000, 220000, 230000, 240000, 250000, 300000 pg/mL or higher). In some embodiments, administration of the bile acid (e.g. TURSO) and the phenylbutyrate compound (e.g. sodium phenylbutyrate) reduces the levels of CSF YKL-40 by about 5000 pg/mL or more (e.g., about 7000, 9000, 11000, 12000, 13000, 14000, 15000, 16000, 18000, 20000, 25000 pg/mL or more).

In some embodiments, prior to treatment the subjects have a baseline CSF IL-15 level of about 1 to about 5 pg/mL (e.g. about 1.5, 2, 2.5, 3, 3.5, 4 or 4.5 pg/mL). In some embodiments, administration of the bile acid (e.g. TURSO) and the phenylbutyrate compound (e.g. sodium phenylbutyrate) reduces the levels of CSF IL-15 by about 0.01 pg/mL or more (e.g., about 0.02, 0.03, 0.04, 0.05, 0.06, 0.07, 0.08, 0.09, 0.1, 0.2, 0.3, 0.4, 0.5 pg/mL or more).

In some embodiments, the CSF Aβ₁₋₄₂ level is about 500 pg/mL or lower (e.g., about 450, 400, 350, 300, 250, 200, 150, 100, 50, or 25 pg/mL, or lower). The subject may have a baseline CSF Aβ₁₋₄₂ level of about 150 to about 550 pg/mL and/or a baseline CSF Aβ₁₋₄₀ level of about 3500 to about 9500 pg/mL. Administration of the bile acid (e.g. TURSO) and the phenylbutyrate compound (e.g. sodium phenylbutyrate) can increase the Aβ₁₋₄₂/Aβ₁₋₄₀ ratio by about 0.001 to about 0.02 (e.g. about 0.002 to about 0.015, or about 0.009).

The subject can have a baseline 8-OHdG level of about 2 to about 5 pg/mL (e.g. about 2.5, 3, 3.5, 4, or 4.5 pg/mL). Administration of the bile acid (e.g. TURSO) and the phenylbutyrate compound (e.g. sodium phenylbutyrate) can increase the 8-OHdG level by about 0.1 pg/mL or more (e.g. about 0.2, 0.3, 0.4, 0.5, 0.6, or 0.7 pg/mL or more).

Methods described in the present disclosure can include treatment of AD per se, as well as treatment for one or more symptoms of AD. “Treating” AD does not require 100% abolition of the disease or disease symptoms in the subject. Any relief or reduction in the severity of symptoms or features of the disease is contemplated. “Treating” AD also refers to a delay in onset of symptoms (e.g., in prophylaxis treatment) or delay in progression of symptoms or the loss of function associated with the disease. “Treating” AD also refers to eliminating or reducing one or more side effects of a treatment (e g. those caused by any of the therapeutic agents for treating AD disclosed herein or known in the art). “Treating” AD also refers to eliminating or reducing one or more direct or indirect effects of AD disease progression. The subject may not exhibit signs of AD but may be at risk for AD. For instance, the subject may carry mutations in genes associated with AD (e.g., carrying one or more copies of the APOEε4 allele), have elevated biomarker levels suggesting a risk of developing AD (e.g., but not limited to, total tau, phospho-tau), or have reduced biomarker levels suggesting a risk of developing AD (e.g., but not limited to, Aβ₁₋₄₂). The subject may exhibit early signs of the disease or display symptoms of established or progressive disease. The disclosure contemplates any degree of delay in the onset of symptoms, alleviation of one or more symptoms of the disease, or delay in the progression of any one or more disease symptoms.

The treatment provided in the present disclosure can be initiated at any stage during disease progression. For example, treatment can be initiated prior to onset (e.g., for subjects at risk for developing AD, for instance, those with elevated total tau or phospho-tau), at symptom onset or immediately following detection of AD symptoms, upon observation of any one or more symptoms (e.g., decline in cognitive functions, memory loss, reduced attention span) that would lead a skilled practitioner to suspect that the subject may be developing AD. Treatment can also be initiated at later stages.

Treatment methods can include a single administration, multiple administrations, and repeating administration as required for the prophylaxis or treatment of AD, or at least one symptom of AD. The duration of prophylaxis treatment can be a single dosage or the treatment may continue (e.g., multiple dosages), e.g., for years or indefinitely for the lifespan of the subject. For example, a subject at risk for AD may be treated with the methods provided herein for days, weeks, months, or even years so as to prevent the disease from occurring or fulminating. In some embodiments treatment methods can include assessing a level of disease in the subject prior to treatment, during treatment, and/or after treatment. The treatment provided herein can be administered one or more times daily, or it can be administered weekly or monthly. In some embodiments, treatment can continue until a decrease in the level of disease in the subject is detected. The methods provided herein may in some embodiments begin to show efficacy (e.g., alleviating one or more symptoms of AD, improvement as measured by a cognitive test, such as, MOCA, ADAS-Cog, DSRS, MADCOMS, FAQ, or NPI-Q) less than 60 days (e.g., less than 50, 45, 40, 35, 30, 25, 20, 15, or 10 days) after the initial administration, or after less than 60 administrations (e.g., less than 50, 45, 40, 35, 30, 25, 20, 15, or 10 administrations).

Methods described in the present disclosure can include treatment of a neurodegenerative disease (e.g., a tauopathy like PSP) per se, as well as treatment for one or more symptoms of a neurodegenerative disease (e.g., a tauopathy like PSP). “Treating” a neurodegenerative disease (e.g., a tauopathy like PSP) does not require 100% abolition of the disease or disease symptoms in the subject. Any relief or reduction in the severity of symptoms or features of the disease is contemplated. “Treating” a neurodegenerative disease (e.g., a tauopathy like PSP) also refers to a delay in onset of symptoms (e.g., in prophylaxis treatment) or delay in progression of symptoms or the loss of function associated with the disease. “Treating” a neurodegenerative disease (e.g., a tauopathy like PSP) also refers to eliminating or reducing one or more side effects of a treatment (e g. those caused by any of the therapeutic agents for treating a neurodegenerative disease (e.g., a tauopathy like PSP) disclosed herein or known in the art). “Treating” a neurodegenerative disease (e.g., a tauopathy like PSP) also refers to eliminating or reducing one or more direct or indirect effects of a neurodegenerative disease (e.g., a tauopathy like PSP) disease progression. The subject may not exhibit signs of a neurodegenerative disease (e.g., a tauopathy like PSP) but may be at risk for a neurodegenerative disease (e.g., a tauopathy like PSP). For instance, the subject may carry mutations in genes associated with a neurodegenerative disease (e.g., a tauopathy like PSP), have elevated biomarker levels suggesting a risk of developing a neurodegenerative disease (e.g., a tauopathy like PSP) (e.g., but not limited to, total tau, phospho-tau, or YKL-40). The subject may exhibit early signs of the disease or display symptoms of established or progressive disease. The disclosure contemplates any degree of delay in the onset of symptoms, alleviation of one or more symptoms of the disease, or delay in the progression of any one or more disease symptoms.

The treatment provided in the present disclosure can be initiated at any stage during disease progression. For example, treatment can be initiated prior to onset (e.g., for subjects at risk for developing a neurodegenerative disease (e.g., a tauopathy like PSP), for instance, those with elevated total tau or phospho-tau), at symptom onset or immediately following detection of a neurodegenerative disease (e.g., a tauopathy like PSP) symptoms, upon observation of any one or more symptoms (e.g., decline in cognitive functions) that would lead a skilled practitioner to suspect that the subject may be developing a neurodegenerative disease (e.g., a tauopathy like PSP). Treatment can also be initiated at later stages.

Treatment methods can include a single administration, multiple administrations, and repeating administration as required for the prophylaxis or treatment of a neurodegenerative disease (e.g., a tauopathy like PSP), or at least one symptom of a neurodegenerative disease (e.g., a tauopathy like PSP). The duration of prophylaxis treatment can be a single dosage or the treatment may continue (e.g., multiple dosages), e.g., for years or indefinitely for the lifespan of the subject. For example, a subject at risk for a neurodegenerative disease (e.g., a tauopathy like PSP) may be treated with the methods provided herein for days, weeks, months, or even years so as to prevent the disease from occurring or fulminating. In some embodiments treatment methods can include assessing a level of disease in the subject prior to treatment, during treatment, and/or after treatment. The treatment provided herein can be administered one or more times daily, or it can be administered weekly or monthly. In some embodiments, treatment can continue until a decrease in the level of disease in the subject is detected. The methods provided herein may in some embodiments begin to show efficacy (e.g., alleviating one or more symptoms of a neurodegenerative disease (e.g., a tauopathy like PSP), improvement as measured by a cognitive test, less than 60 days (e.g., less than 50, 45, 40, 35, 30, 25, 20, 15, or 10 days) after the initial administration, or after less than 60 administrations (e.g., less than 50, 45, 40, 35, 30, 25, 20, 15, or 10 administrations).

The terms “administer”, “administering”, or “administration” as used herein refers to administering drugs described herein to a subject using any art-known method, e.g., ingesting, injecting, implanting, absorbing, or inhaling, the drug, regardless of form. In some embodiments, one or more of the compounds disclosed herein can be administered to a subject by ingestion orally and/or topically (e.g., nasally). For example, the methods herein include administration of an effective amount of compound or compound composition to achieve the desired or stated effect Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Following administration, the subject can be evaluated to detect, assess, or determine their level of disease. In some embodiments, treatment can continue until a change (e.g., reduction) in the level of disease in the subject is detected.

Upon improvement of a patient's condition (e.g., a change (e.g., decrease) in the level of disease in the subject), a maintenance dose of a compound, composition or combination of this disclosure may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

### Symptom and Outcome Measurements

Methods of evaluating symptoms, monitoring a neurodegenerative disease, such as AD or PSP, progression and/or evaluating the subject's response to the treatment methods are described herein. Non-limiting examples include physical evaluation by a physician, cognitive tests (e.g., ADAS-Cog, MoCA, DSRS, MADCOMS, FAQ, NPI-Q, MDS PSP Diagnostic Criteria, PSP rating scale, or other appropriate test depending on the neurodegenerative disease), measurement of one or more CSF biomarkers (e.g., total tau (t-tau), phospho-tau 181 (e.g., p-tau 181 or another phospho-tau), neurofilament-light (NfL), Ubiquitin carboxyl-terminal hydrolase L1 (UCHL1)/PGP9.5, Glial fibrillary acidic protein (GFAP), 8-hydoxy-2′-deoxyguanosine (8-OHdG), Soluble insulin receptor (SIR), Aβ₁₋₄₂, Aβ₁₋₁₀, Aβ₁₋₄₂/Aβ₁₋₁₀ ratio, leptin, 24-hydroxycholesterol, YKL-40), neuroimaging (e.g., measuring hippocampal volume, grey matter, average cortical thickness, number of white matter lesions, white matter lesion volume, ventricular volume through known methods, such as, MRI, CT, SPECT, FDG-PET, or DIT), or a combination of any of these methods (e.g., combination of a cognitive test and the level of one or more CSF biomarker).

In some embodiments, the methods described herein result in an improvement in score received in one or more cognitive test. In other embodiments, the methods described herein result in a significant decrease in t-tau, phospho-tau, or YKL-40 (for example, as measured in the CSF). In another example, the methods described herein result in an increase in Aβ₁₋₄₂/Aβ₁₋₄₀ (for example, as measured in the CSF; see e.g., Lewczuk P, Lelental N, Spitzer P, Maler J M, Komhuber J. Amyloid-β 42/40 cerebrospinal fluid concentration ratio in the diagnostics of Alzheimer's disease: validation of two novel assays. J Alzheimers Dis. 2015; 43 (1): 183-91. doi: 10.3233/JAD-140771. PMID: 25079805; Hansson, O., Lehmann, S., Otto, M. et al. Advantages and disadvantages of the use of the CSF Amyloid β (Aβ) 42/40 ratio in the diagnosis of Alzheimer's Disease. Alz Res Therapy 11, 34 (2019). https://doi.org/10.1186/s13195-019-0485-0 and James D. Doecke, Virginia Pérez-Grijalba, Noelia Fandos, Christopher Fowler, Victor L. Villemagne, Colin L. Masters, Pedro Pesini, Manuel Sarasa, for the AIBL Research Group, “Total Aβ42/Aβ40 ratio in plasma predicts amyloid-PET status, independent of clinical AD diagnosis,” Neurology April 2020, 94 (15) e1580-e1591; DOI: 10.1212/WNL.0000000000009240; incorporated herein by reference). In some embodiments, the methods described herein result in a significant increase in ventricular volume (for example, as measured by an imaging method, such as, MRI). In some embodiments, the methods described herein result in an increase in hippocampal volume, grey matter, average cortical thickness, and/or a decrease in the number of white matter lesions (for example, as measured by an imaging method, such as, MRI).

### Composition

The present disclosure provides methods of treating at least one symptom of a neurodegenerative disease (such as AD or PSP) in a subject, the methods including administering to the subject a bile acid or a pharmaceutically acceptable salt thereof and a phenylbutyrate compound. In some embodiments, the methods include administering a composition comprising a TURSO and a sodium phenylbutyrate to a subject.

### Bile Acid

As used herein, “bile acid” refers to naturally occurring surfactants having a nucleus derived from cholanic acid substituted with a 3α-hydroxyl group and optionally with other hydroxyl groups as well, typically at the C6, C7 or C12 position of the sterol nucleus. Bile acid derivatives (e.g., aqueous soluble bile acid derivatives) and bile acids conjugated with an amine are also encompassed by the term “bile acid”. Bile acid derivatives include, but are not limited to, derivatives formed at the carbon atoms to which hydroxyl and carboxylic acid groups of the bile acid are attached with other functional groups, including but not limited to halogens and amino groups. Soluble bile acids may include an aqueous preparation of a free acid form of bile acids combined with one of HCl, phosphoric acid, citric acid, acetic acid, ammonia, or arginine. Suitable bile acids include but are not limited to, taurursodiol (TURSO), ursodeoxycholic acid (UDCA), chenodeoxycholic acid (also referred to as “chenodiol” or “chenic acid”), cholic acid, hyodeoxycholic acid, deoxycholic acid, 7-oxolithocholic acid, lithocholic acid, iododeoxycholic acid, iocholic acid, taurochenodeoxycholic acid, taurodeoxycholic acid, glycoursodeoxycholic acid, taurocholic acid, glycocholic acid, or an analog, derivative, or prodrug thereof.

In some embodiments, the bile acids of the present disclosure are hydrophilic bile acids. Hydrophilic bile acids include but are not limited to, TURSO, UDCA, chenodeoxycholic acid, cholic acid, hyodeoxycholic acid, lithocholic acid, and glycoursodeoxycholic acid. Pharmaceutically acceptable salts or solvates of any of the bile acids disclosed herein are also contemplated. In some embodiments, bases commonly employed to form pharmaceutically acceptable salts of the bile acids of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The terms “tauroursodeoxycholic acid” (TUDCA) and “taurursodiol” (TURSO) are used interchangeably herein.

The bile acid described herein can be TURSO, as shown in formula I (with labeled carbons to assist in understanding where substitutions may be made). In some embodiments, the TURSO is a hydrate, such as TURSO dihydrate.

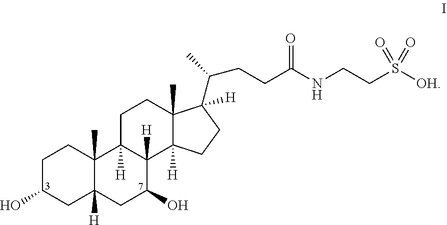

The bile acid described herein can be UDCA as shown in formula II (with labeled carbons to assist in understanding where substitutions may be made).

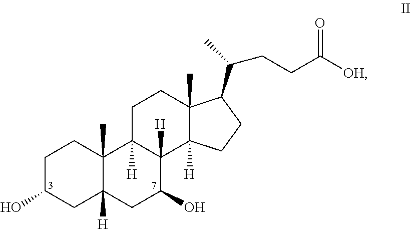

or a pharmaceutically acceptable salt thereof.

Derivatives of bile acids of the present disclosure can be physiologically related bile acid derivatives. For example, any combination of substitutions of hydrogen at position 3 or 7, a shift in the stereochemistry of the hydroxyl group at positions 3 or 7, in the formula of TURSO or UDCA are suitable for use in the present composition.

The “bile acid” can also be a bile acid conjugated with an amino acid. The amino acid in the conjugate can be, but are not limited to, taurine, glycine, glutamine, asparagine, methionine, or carbocysteine. Other amino acids that can be conjugated with a bile acid of the present disclosure include arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid. One example of such a bile acid is a compound of formula III:

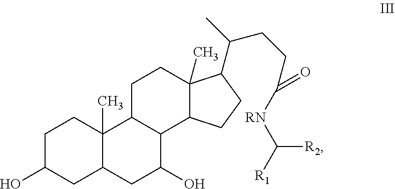

wherein
R is —H or C₁-C₄ alkyl;
R₁ is —CH₂—SO₃R₃, CH₂COOH, or CH₂CH₂COOH, and R₂ is —H;
or R₁ is —COOH and R₂ is —CH₂—CH₂—CONH₂, —CH₂—CONH₂, —CH₂—CH₂—SCH₃, CH₂CH₂CH₂NH(C═NH)NH₂, CH₂ (imidazolyl), CH₂CH₂CH₂CH₂NH₂, CH₂COOH, CH₂CH₂COOH, CH₂OH, CH(OH)CH₃, CH₂SH, pyrrolidin-2-yl, CH₃, 2-propyl, 2-butyl, 2-methylbutyl, CH₂ (phenyl), CH₂ (4-OH-phenyl), or —CH₂—S—CH₂—COOH; and
R₃ is —H or the residue of an amino acid, or a pharmaceutically acceptable analog, derivative, prodrug thereof, or a mixture thereof. One example of the amino acid is a basic amino acid. Other examples of the amino acid include glycine, glutamine, asparagine, methionine, carbocysteine, arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid.

Another example of a bile acid of the present disclosure is a compound of formula IV:

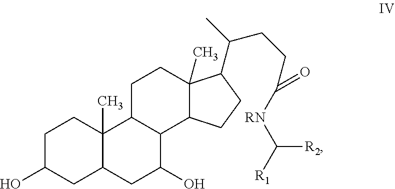

wherein
   R is —H or C₁-C₄ alkyl;
   R₁ is —CH₂—SO₃R₃, and R₂ is —H;
   or R₁ is —COOH and R₂ is —CH₂—CH₂—CONH₂, —CH₂—CONH₂, —CH₂—CH₂—SCH₃, or —CH₂—S—CH₂—COOH; and
   R₃ is —H or the residue of a basic amino acid, or a pharmaceutically acceptable analog, derivative, prodrug thereof, or a mixture thereof. Examples of basic amino acids include lysine, histidine, and arginine.

In some embodiments, the bile acid is TURSO. TURSO is an amphiphilic bile acid and is the taurine conjugate form of UDCA. TURSO recovers mitochondrial bioenergetic deficits through incorporating into the mitochondrial membrane, reducing Bax translocation to the mitochondrial membrane, reducing mitochondrial permeability, and increasing the apoptotic threshold of the cell (Rodrigues et al. Biochemistry 42, 10:3070-3080, 2003). It is used for the treatment of cholesterol gallstones, where long periods of treatment is generally required (e.g., 1 to 2 years) to obtain complete dissolution. It has been used for the treatment of cholestatic liver diseases including primary cirrhosis, pediatric familial intrahepatic cholestasis and primary sclerosing cholangitis and cholestasis due to cystic fibrosis. TURSO is contraindicated in subjects with biliary tract infections, frequent biliary colic, or in subjects who have trouble absorbing bile acids (e.g. ileal disease or resection). Drug interactions may include with substances that inhibit the absorption of bile acids, such as cholestyramine, and with drugs that increase the elimination of cholesterol in the bile (TURSO reduces biliary cholesterol content). Based on similar physicochemical characteristics, similarities in drug toxicity and interactions exist between TURSO and UDCA. The most common adverse reactions reported with the use of TURSO (≥1%) are: abdominal discomfort, abdominal pain, diarrhea, nausea, pruritus, and rash. There are some cases of pruritus and a limited number of cases of elevated liver enzymes.

In some embodiments, the bile acid is UDCA. UDCA, or ursodiol, has been used for treating gallstones, and is produced and secreted endogenously by the liver as a taurine (TURSO) or glycine (GUDCA) conjugate. Taurine conjugation increases the solubility of UDCA by making it more hydrophilic. TURSO is taken up in the distal ileum under active transport and therefore likely has a slightly a longer dwell time within the intestine than UDCA which is taken up more proximally in the ileum. Ursodiol therapy has not been associated with liver damage. Abnormalities in liver enzymes have not been associated with Actigall® (Ursodiol USP capsules) therapy and, Actigall® has been shown to decrease liver enzyme levels in liver disease. However, subjects given Actigall® should have SGOT (AST) and SGPT (ALT) measured at the initiation of therapy and thereafter as indicated by the particular clinical circumstances. Previous studies have shown that bile acid sequestering agents such as cholestyramine and colestipol may interfere with the action of ursodiol by reducing its absorption. Aluminum-based antacids have been shown to adsorb bile acids in vitro and may be expected to interfere with ursodiol in the same manner as the bile acid sequestering agents. Estrogens, oral contraceptives, and clofibrate (and perhaps other lipid-lowering drugs) increase hepatic cholesterol secretion, and encourage cholesterol gallstone formation and hence may counteract the effectiveness of ursodiol.

### Phenylbutyrate Compounds

Phenylbutyrate compound is defined herein as encompassing phenylbutyrate (a low molecular weight aromatic carboxylic acid) as a free acid (4-phenylbutyrate (4-PBA), 4-phenylbutyric acid, or phenylbutyric acid), and pharmaceutically acceptable salts, co-crystals, polymorphs, hydrates, solvates, conjugates, derivatives or pro-drugs thereof. Phenylbutyrate compounds described herein also encompass analogs of 4-PBA, including but not limited to Glyceryl Tri-(4-phenylbutyrate), phenylacetic acid (which is the active metabolite of PBA), 2-(4-Methoxyphenoxy) acetic acid (2-POAA-OMe), 2-(4-Nitrophenoxy) acetic acid (2-POAA-NO2), and 2-(2-Naphthyloxy) acetic acid (2-NOAA), and their pharmaceutically acceptable salts. Phenylbutyrate compounds also encompass physiologically related 4-PBA species, such as but not limited to any substitutions for Hydrogens with Deuterium in the structure of 4-PBA. Other HDAC2 inhibitors are contemplated herein as substitutes for phenylbutyrate compounds.

Physiologically acceptable salts of phenylbutyrate, include, for example sodium, potassium, magnesium or calcium salts. Other example of salts include ammonium, zinc, or lithium salts, or salts of phenylbutyrate with an orgain amine, such as lysine or arginine.

In some embodiments of any of the methods described herein, the phenylbutyrate compound is sodium phenylbutyrate. Sodium phenylbutyrate has the following formula:

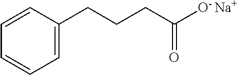

Phenylbutyrate is a pan-HDAC inhibitor and can ameliorate ER stress through upregulation of the master chaperone regulator DJ-1 and through recruitment of other chaperone proteins (See e.g., Zhou et al. J Biol Chem 286:14941-14951, 2011 and Suaud et al. JBC. 286:21239-21253, 2011). The large increase in chaperone production reduces activation of canonical ER stress pathways, folds misfolded proteins, and has been shown to increase survival in in vivo models including the G93A SOD1 mouse model of ALS (See e.g., Ryu, H et al. J Neurochem. 93:1087-1098, 2005).

### Formulation

Bile acids and phenylbutyrate compounds described herein can be formulated for use as or in pharmaceutical compositions. For example, the methods described herein can include administering an effective amount of a composition comprising TURSO and sodium phenylbutyrate. The term “effective amount”, as used herein, refer to an amount or a concentration of one or more drugs administered for a period of time (including acute or chronic administration and periodic or continuous administration) that is effective within the context of its administration for causing an intended effect or physiological outcome. The composition can include about 5% to about 15% w/w (e.g., about 6% to about 14%, about 7% to about 13%, about 8% to about 12%, about 8% to about 11%, about 9% to about 10%, or about 9.7% w/w) of TURSO and about 15% to about 45% w/w (e.g., about 20% to about 40%, about 25% to about 35%, about 28% to about 32%, or about 29% to about 30%, e.g., about 29.2% w/w) of sodium phenylbutyrate. In some embodiments, the composition includes about 9.7% w/w of TURSO and 29.2% w/w of sodium phenylbutyrate.

The sodium phenylbutyrate and TURSO can be present in the composition at a ratio by weight of between about 1:1 to about 4:1 (e.g., about 2:1 or about 3:1). In some embodiments, the ratio between sodium phenylbutyrate and TURSO is about 3:1.

The compositions described herein can include any pharmaceutically acceptable carrier, adjuvant, and/or vehicle. The term “pharmaceutically acceptable carrier or adjuvant” refers to a carrier or adjuvant that may be administered to a patient, together with a compound disclosed herein, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

Compositions of the present disclosure can include about 8% to about 24% w/w of dextrates (e.g., about 9% to about 23%, about 10% to about 22%, about 10% to about 20%, about 11% to about 21%, about 12% to about 20%, about 13% to about 19%, about 14% to about 18%, about 14% to about 17%, about 15% to about 16%, or about 15.6% w/w of dextrates). Both anhydrous and hydrated dextrates are contemplated herein. The dextrates of the present disclosure can include a mixture of saccharides developed from controlled enzymatic hydrolysis of starch. Some embodiments of any of the compositions described herein include hydrated dextrates (e.g., NF grade, obtained from JRS Pharma, Colonial Scientific, or Quadra).

Compositions of the present disclosure can include about 1% to about 6% w/w of sugar alcohol (e.g., about 2% to about 5%, about 3% to about 4%, or about 3.9% w/w of sugar alcohol). Sugar alcohols can be derived from sugars and contain one hydroxyl group (—OH) attached to each carbon atom. Both disaccharides and monosaccharides can form sugar alcohols. Sugar alcohols can be natural or produced by hydrogenation of sugars. Exemplary sugar alcohols include but are not limited to, sorbitol, xylitol, and mannitol. In some embodiments, the composition comprises about 1% to about 6% w/w (e.g., about 2% to about 5%, about 3% to about 4%, or about 3.9% w/w) of sorbitol.

Compositions of the present disclosure can include about 22% to about 35% w/w of maltodextrin (e.g., about 22% to about 33%, about 24% to about 31%, about 25% to about 32%, about 26% to about 30%, or about 28% to about 29% w/w, e.g., about 28.3% w/w of maltodextrin). Maltodextrin can form a flexible helix enabling the entrapment of the active ingredients (e.g., any of the phenylbutyrate compounds and bile acids described herein) when solubilized into solution, thereby masking the taste of the active ingredients. Maltodextrin produced from any suitable sources are contemplated herein, including but not limited to, pea, rice, tapioca, corn, and potato. In some embodiments, the maltodextrin is pea maltodextrin. In some embodiments, the composition includes about 28.3% w/w of pea maltodextrin. For example, pea maltodextrin obtained from Roquette (KLEPTOSE® LINECAPS) can be used.

The compositions described herein can further include sugar substitutes (e.g. sucralose). For example, the compositions can include about 0.5% to about 5% w/w of sucralose (e.g., about 1% to about 4%, about 1% to about 3%, or about 1% to about 2%, e.g., about 1.9% w/w of sucralose). Other sugar substitutes contemplated herein include but are not limited to aspartame, neotame, acesulfame potassium, saccharin, and advantame.

In some embodiments, the compositions include one or more flavorants. The compositions can include about 2% to about 15% w/w of flavorants (e.g., about 3% to about 13%, about 3% to about 12%, about 4% to about 9%, about 5% to about 10%, or about 5% to about 8%, e.g., about 7.3% w/w) Flavorants can include substances that give another substance flavor, or alter the characteristics of a composition by affecting its taste. Flavorants can be used to mask unpleasant tastes without affecting physical and chemical stability, and can be selected based on the taste of the drug to be incorporated. Suitable flavorants include but are not limited to natural flavoring substances, artificial flavoring substances, and imitation flavors. Blends of flavorants can also be used. For example, the compositions described herein can include two or more (e.g., two, three, four, five or more) flavorants. Flavorants can be soluble and stable in water. Selection of suitable flavorants can be based on taste testing. For example, multiple different flavorants can be added to a composition separately, which are subjected to taste testing. Exemplary flavorants include any fruit flavor powder (e.g., peach, strawberry, mango, orange, apple, grape, raspberry, cherry or mixed berry flavor powder). The compositions described herein can include about 0.5% to about 1.5% w/w (e.g., about 1% w/w) of a mixed berry flavor powder and/or about 5% to about 7% w/w (e.g., about 6.3% w/w) of a masking flavor. Suitable masking flavors can be obtained from e.g., Firmenich.

The compositions described herein can further include silicon dioxide (or silica). Addition of silica to the composition can prevent or reduce agglomeration of the components of the composition. Silica can serve as an anti-caking agent, adsorbent, disintegrant, or glidant. In some embodiments, the compositions described herein include about 0.1% to about 2% w/w of porous silica (e.g., about 0.3% to about 1.5%, about 0.5% to about 1.2%, or about 0.8% to about 1%, e.g., 0.9% w/w). Porous silica may have a higher H₂O absorption capacity and/or a higher porosity as compared to fumed silica, at a relative humidity of about 20% or higher (e.g., about 25%, 30%, 35%, 40%, 45%, 50%, 55%, 60%, 65%, 70%, 75%, 80%, 85%, 90%, or 95% or higher). The porous silica can have an H₂O absorption capacity of about 5% to about 40% (e.g. about 20% to about 40%, or about 30% to about 40%) by weight at a relative humidity of about 50%. The porous silica can have a higher porosity at a relative humidity of about 20% or higher (e.g., about 30%, 40%, 50%, 60%, 70%, 80%, 90% or higher) as compared to that of fumed silica. In some embodiments, the porous silica have an average particle size of about 2 μm to about 10 μm (e.g. about 3 μm to about 9 μm, about 4 μm to about 8 μm, about 5 μm to about 8 μm, or about 7.5 μm). In some embodiments, the porous silica have an average pore volume of about 0.1 cc/gm to about 2.0 cc/gm (e.g., about 0.1 cc/gm to about 1.5 cc/gm, about 0.1 cc/gm to about 1 cc/gm, about 0.2 cc/gm to about 0.8 cc/gm, about 0.3 cc/gm to about 0.6 cc/gm, or about 0.4 cc/gm). In some embodiments, the porous silica have a bulk density of about 50 g/L to about 700 g/L (e.g. about 100 g/L to about 600 g/L, about 200 g/L to about 600 g/L, about 400 g/L to about 600 g/L, about 500 g/L to about 600 g/L, about 540 g/L to about 580 g/L, or about 560 g/L). In some embodiments, the compositions described herein include about 0.05% to about 2% w/w (e.g., any subranges of this range described herein) of Syloid® 63FP (WR Grace).

The compositions described herein can further include one or more buffering agents. For example, the compositions can include about 0.5% to about 5% w/w of buffering agents (e.g., about 1% to about 4% w/w, about 1.5% to about 3.5% w/w, or about 2% to about 3% w/w, e.g. about 2.7% w/w of buffering agents). Buffering agents can include weak acid or base that maintain the acidity or pH of a composition near a chosen value after addition of another acid or base. Suitable buffering agents are known in the art. In some embodiments, the buffering agent in the composition provided herein is a phosphate, such as a sodium phosphate (e.g., sodium phosphate dibasic anhydrous). For example, the composition can include about 2.7% w/w of sodium phosphate dibasic.

The compositions can also include one or more lubricants. For example, the compositions can include about 0.05% to about 1% w/w of lubricants (e.g., about 0.1% to about 0.9%, about 0.2% to about 0.8%, about 0.3% to about 0.7%, or about 0.4% to about 0.6%, e.g. about 0.5% w/w of lubricants). Exemplary lubricants include, but are not limited to sodium stearyl fumarate, magnesium stearate, stearic acid, metallic stearates, talc, waxes and glycerides with high melting temperatures, colloidal silica, polyethylene glycols, alkyl sulphates, glyceryl behenate, and hydrogenated oil. Additional lubricants are known in the art. In some embodiments, the composition includes about 0.05% to about 1% w/w (e.g., any of the subranges of this range described herein) of sodium stearyl fumarate. For example, the composition can include about 0.5% w/w of sodium stearyl fumarate.

In some embodiments, the composition include about 29.2% w/w of sodium phenylbutyrate, about 9.7% w/w of TURSO, about 15.6% w/w of dextrates, about 3.9% w/w of sorbitol, about 1.9% w/w of sucralose, about 28.3% w/w of maltodextrin, about 7.3% w/w of flavorants, about 0.9% w/w of silicon dioxide, about 2.7% w/w of sodium phosphate (e.g. sodium phosphate dibasic), and about 0.5% w/w of sodium stearyl fumerate.

The composition can include about 3000 mg of sodium phenylbutyrate, about 1000 mg of TURSO, about 1600 mg of dextrates, about 400 mg of sorbitol, about 200 mg of sucralose, about 97.2 mg of silicon dioxide, about 2916 mg of maltodextrin, about 746 mg of flavorants (e.g. about 102 mg of mixed berry flavor and about 644 mg of masking flavor), about 280 mg of sodium phosphate (e.g. sodium phosphate dibasic), and about 48.6 mg of sodium stearyl fumerate.

Additional suitable sweeteners or taste masking agents can also be included in the compositions, such as but not limited to, xylose, ribose, glucose, mannose, galactose, fructose, dextrose, sucrose, maltose, steviol glycosides, partially hydrolyzed starch, and corn syrup solid. Water soluble artificial sweeteners are contemplated herein, such as the soluble saccharin salts (e.g., sodium or calcium saccharin salts), cyclamate salts, acesulfam potassium (acesulfame K), and the free acid form of saccharin and aspartame based sweeteners such as L-aspartyl-phenylalanine methyl ester, Alitame® or Neotame®. The amount of sweetener or taste masking agents can vary with the desired amount of sweeteners or taste masking agents selected for a particular final composition.

Pharmaceutically acceptable binders in addition to those described above are also contemplated. Examples include cellulose derivatives including microcrystalline cellulose, low-substituted hydroxypropyl cellulose (e.g. LH 22, LH 21, LH 20, LH 32, LH 31, LH30); croscarmellose starches, including potato starch; sodium (i.e. cross-linked carboxymethylcellulose sodium salt; e.g. Ac-Di-Sol®); alginic acid or alginates; insoluble polyvinylpyrrolidone (e.g. Polyvidon® CL, Polyvidon® CL-M, Kollidon® CL, Polyplasdone® XL, Polyplasdone® XL-10); and sodium carboxymethyl starch (e.g. Primogel® and Explotab®).

Additional fillers, diluents or binders may be incorporated such as polyols, sucrose, sorbitol, mannitol, Erythritol®, Tagatose®, lactose (e.g., spray-dried lactose, α-lactose, β-lactose, Tabletose®, various grades of Pharmatose®, Microtose or Fast-Floc®), microcrystalline cellulose (e.g., various grades of Avicel®, such as Avicel® PH101, Avicel® PH102 or Avicel® PH105, Elcema® P100, Emcocel®, Vivacel®, Ming Tai® and Solka-Floc®), hydroxypropylcellulose, L-hydroxypropylcellulose (low-substituted) (e.g. L-HPC-CH₃₁, L-HPC-LH11, LH 22, LH 21, LH 20, LH 32, LH 31, LH30), dextrins, maltodextrins (e.g. Lodex® 5 and Lodex® 10), starches or modified starches (including potato starch, maize starch and rice starch), sodium chloride, sodium phosphate, calcium sulfate, and calcium carbonate.

The compositions described herein can be formulated or adapted for administration to a subject via any route (e.g. any route approved by the Food and Drug Administration (FDA)). Exemplary methods are described in the FDA's CDER Data Standards Manual, version number 004 (which is available at fda.give/cder/dsm/DRG/drg00301.html).

Pharmaceutical compositions are typically formulated to be compatible with its intended route of administration. Examples of routes of administration include parenteral (subcutaneous, intracutaneous, intravenous, intradermal, intramuscular, intra-articular, intraarterial, intrasynovial, intrasternal, intrathecal, intralesional and intracranial injection or infusion techniques), oral (e.g., inhalation or through a feeding tube), transdermal (topical), transmucosal, and rectal administration.

Pharmaceutical compositions can be in the form of a solution or powder for inhalation and/or nasal administration. In some embodiments, the pharmaceutical composition is formulated as a powder filled sachet. Suitable powders may include those that are substantially soluble in water. Pharmaceutical compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions can be orally administered in any orally acceptable dosage form including, but not limited to, powders, capsules, tablets, emulsions and aqueous suspensions, dispersions and solutions. In the case of powders for oral administration, the powders can be substantially dissolved in water prior to administration. In the case of tablets for oral use, carriers which are commonly used include lactose and corn starch. Lubricating agents, such as magnesium stearate, may be added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, the compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some embodiments, therapeutic compositions disclosed herein can be formulated for sale in the US, imported into the US, and/or exported from the US. The pharmaceutical compositions can be included in a container, pack, or dispenser together with instructions for administration. In some embodiments, the invention provides kits that include the bile acid and phenylbutyrate compounds. The kit may also include instructions for the physician and/or patient, syringes, needles, box, bottles, vials, etc.

### Additional Therapeutic Agents and Further Combination Treatments

Any of the pharmaceutical compositions or methods described herein can further include one or more additional therapeutic agents in amounts effective for treating or achieving a modulation of at least one symptom of AD. Any known AD therapeutic agents known in the art can be used as an additional therapeutic agent. Exemplary therapeutic agents can also include acetylcholinesterase inhibitors. Exemplary therapeutic agents include tacrine (Cognex®), rivastigmine (Exelon®), galantamine (Nivalin® and Razadyne®), donepezil (Aricept®), and memantine (Namenda®). Any known antidepressants are contemplated herein, including but not limited to selective serotonin inhibitors, serotonin-norepinephrine reuptake inhibitors, serotonin modulators and stimulators, serotonin antagonists and reuptake inhibitors, norepinephrine reuptake inhibitors, norepinephrine-dopamine reuptake inhibitors, tricyclic antidepressants, tetracyclic antidepressants, monoamine oxidase inhibitors, and NMDA receptor antagonists.

The methods of the present disclosure can include administering to a subject one or more additional therapeutic agents (e.g., any of the additional therapeutic agents disclosed herein or known in the art), in combination with a bile acid (e.g. any of the suitable bile acids described herein) or a pharmaceutically acceptable salt thereof and a phenylbutyrate compound (e.g., any of the suitable phenylbutyrate compounds described herein). The additional therapeutic agent(s) can be administered for a period of time before administering the initial dose of a composition comprising a bile acid or a pharmaceutically acceptable salt thereof (e.g., TURSO) and a phenylbutyrate compound (e.g., sodium phenylbutyrate), and/or for a period of time after administering the final dose of the composition. In some embodiments, a subject in the methods described herein has been previously treated with one or more additional therapeutic agents (e.g., any of the additional therapeutic agents described herein, such as Donepezil, Galantamine, Rivastigamine, Memantine). In some embodiments, the subject has been administered a stable dose of the therapeutic agent(s) (e.g., Donepezil, Galantamine, Rivastigamine, and/or Memantine) for at least 30 days (e.g., at least 40 days, 50 days, 60 days, 90 days, or 120 days) prior to administering the composition of the present disclosure. In some embodiments, treatment with a composition comprising a bile acid or a pharmaceutically acceptable salt thereof (e.g., TURSO) and a phenylbutyrate compound (e.g., sodium phenylbutyrate), as described herein, may help in the reducing the need for treatment with one or more additional therapeutic agents (e.g., Donepezil, Galantamine, Rivastigamine, Memantine).

Any of the pharmaceutical compositions or methods described herein can further include one or more additional therapeutic agents in amounts effective for treating or achieving a modulation of at least one symptom of PSP. These therapies include therapies approved for treating progressive supranuclear palsy and those in development for treating progressive supranuclear palsy. These therapies include, but are not limited to, medications to treat movement disorders, medications to treat psychiatric disorders, psychotherapy, speech therapy, physical therapy, and occupational therapy. Medications to treat movement disorders include, but are not limited to, tetrabenazine, antipsychotic drugs, such as haloperidol, chlorpromazine, risperidone, and quetiapine, and other medications such as amantadine, levetiracetam, and, clonazepam. Medications to treat psychiatric disorders include, but are not limited to, antidepressants such as citalopram, fluoxetine, and sertraline, antipsychotic drugs such as quetiapine, risperidone, and olanzapine, and mood-stabilizing drugs, including anticonvulsants, such as valproate, carbamazepine, and lamotrigine. Psychotherapy includes, but is not limited to, talk therapy to help a subject manage behavioral problems, depression, and suicidal thoughts. Speech therapy includes, but is not limited to, improving a subjects ability to speak clearly, and improve function and control of muscles used for eating and swallowing. Physical therapy includes, but is not limited to, enhancing strength, flexibility, balance and coordination, reducing the risk of falls, and improve posture to lessen the severity of movement problems. Occupational therapy includes, but is not limited to, use of assistive devices that improve functional abilities such as handrails, and eating and drinking utensils for subjects with diminished motor skills.

The combination of a bile acid or a pharmaceutically acceptable salt thereof, a phenylbutyrate compound, and one or more additional therapeutic agents can have a synergistic effect in treating a neurodegenerative disease (e.g., PSP or AD). Smaller doses of the additional therapeutic agents may be required to obtain the same pharmacological effect, when administered in combination with a bile acid or a pharmaceutically acceptable salt thereof, and a phenylbutyrate compound. In some embodiments, the amount of the additional therapeutic agent(s) administered in combination with a bile acid or a pharmaceutically acceptable salt thereof and a phenylbutyrate compound can be reduced by at least about 10% (e.g., at least about 15%, 20%, 25%, 30%, 35%, 40%, 45%, 50%, or 55%) compared to the dosage amount used when the additional therapeutic agent(s) is administered alone. Additionally or alternatively, the methods of the present disclosure can reduce the required frequency of administration of other therapeutic agents (e.g., other AD therapeutic agents) to obtain the same pharmacological effect.

Some embodiments of the present disclosure provide a method of treating at least one symptom of AD or preventing the onset of AD in a human subject, the method comprising administering to the human subject an effective amount of (a) a bile acid or a pharmaceutically acceptable salt thereof (e.g., any of the bile acid or a pharmaceutically acceptable salt thereof described herein); (b) a phenylbutyrate compound (e.g., any of the phenylbutyrate compounds described herein); and (c) one of the additional therapeutic AD agents listed above, to thereby treat at least one symptom of AD or prevent the onset of AD in the human subject.

The bile acid or a pharmaceutically acceptable salt thereof and the phenylbutyrate compound can be administered shortly after a meal (e.g., within two hours of a meal) or under fasting conditions. The subject may have consumed food items (e.g., solid foods or liquid foods) less than 2 hours before administration of a bile acid or a pharmaceutically acceptable salt thereof and/or a phenylbutyrate compound; or will consume food items less than 2 hours after administration of one or both of the compounds. Food items may affect the rate and extent of absorption of the bile acid or a pharmaceutically acceptable salt thereof and/or the phenylbutyrate compound. For instance, food can change the bioavailability of the compounds by delaying gastric emptying, stimulating bile flow, changing gastrointestinal pH, increasing splanchnic blood flow, changing luminal metabolism of the substance, or physically or chemically interacting with a dosage form or the substance. The nutrient and caloric contents of the meal, the meal volume, and the meal temperature can cause physiological changes in the GI tract in a way that affects drug transit time, luminal dissolution, drug permeability, and systemic availability. In general, meals that are high in total calories and fat content are more likely to affect the GI physiology and thereby result in a larger effect on the bioavailability of a drug. The methods provided herein can further include administering to the subject a plurality of food items, for example, less than 2 hours (e.g., less than 1.5 hour, 1 hour, or 0.5 hour) before or after administering the bile acid or a pharmaceutically acceptable salt thereof, and/or the phenylbutyrate compound.

### EXAMPLES

Additional embodiments are disclosed in further detail in the following examples, which are provided by way of illustration and are not in any way intended to limit the scope of this disclosure or the claims.

### Example 1: Evaluation of the Safety, Tolerability, Efficacy and Activity of AMX0035, a Fixed Combination of Phenylbutyrate (PB) and Tauroursodeoxycholic Acid (TUDCA), for Treatment of AD

### 1. Summary

### AMX0035 Rationale

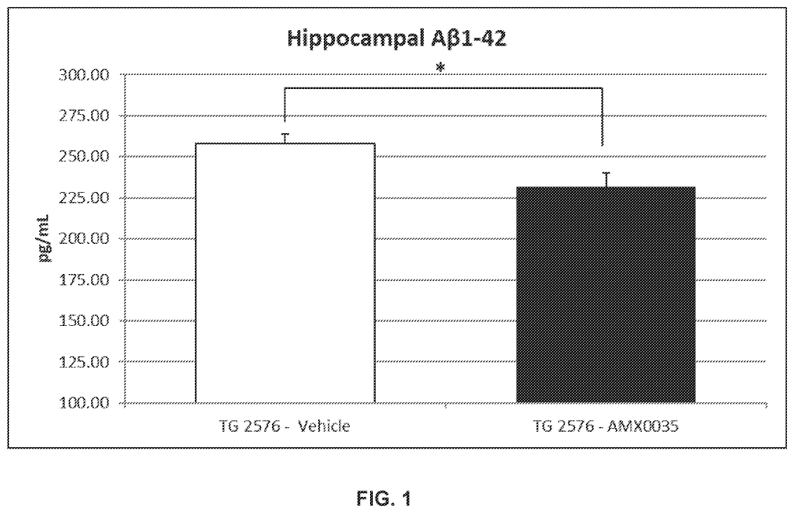

AMX0035 was tested in an acute, pre-plaque Tg2576 mouse model. As shown in FIG. 1, AMX0035 treatment significantly reduced soluble amyloid beta, preferentially Aβ42.

### 1.1 Study Objectives

This study was intended as a proof of concept of AMX0035 as a safe and effective treatment of adult subjects with AD. The primary objectives of the study are:
To compare the safety and tolerability of a fixed-dose combination of AMX0035 (a TUDCA/PB combination) versus placebo in subjects with MCI (high or intermediate likelihood due to AD) or dementia due to AD over an approximate 24-week treatment period;
To determine the effects of AMX0035 treatment on AD progression as measured by a global statistical test (GST)

The secondary objectives of the study are:
To determine the effects of AMX0035 treatment on whole brain and regional brain atrophy, as assessed by volumetric Magnetic Resonance Imaging (vMRI);
To measure the effects of treatment on functional MRI measures including connectivity with resting state BOLD;
To assess the impact of AMX0035 on clinical symptoms as measured by ADAS-Cog. DSRS, FAQ, and additional clinical outcomes;
To assess the effect of AMX0035 on measures of neuropsychiatric symptoms, as assessed by the Neuropsychiatric Inventory Questionnaire (NPI-Q)

The exploratory objectives of the study are:
To measure the effect of AMX0035 on biochemical markers of neurosynaptic damage, mitochondrial redox, amyloid-B142, tau, and neuroinflammation, as assessed in cerebrospinal fluid (CSF) from all volunteers.

The primary estimand is the effect of treatment as measured by a change from baseline in GST (comprised of Mild/Moderate AD Composite Scale (MADCOMS), FAQ, and hippocampal volume) estimated at 24 weeks relative to placebo in individuals with MCI (high or intermediate likelihood due to AD) or dementia due to AD. The study population for the primary estimand was the intent to treat population.

### 1.2 Study Outcome Measures

### 1.2.1 Primary Outcome Measures

The primary outcome measures for the study included safety and tolerability of AMX0035 in Alzheimer's disease.

Adverse events (AE), symptomatic, physical exam, neurological exam, and laboratory parameters were collected prospectively to monitor the safety and tolerability of study drugs. Safety and tolerability were assessed by the procedures outlined in the section “Study Schedule”.

### 1.2.2 Secondary Outcome Measures

The secondary outcome measures included neurophysiological biomarker assessments and cognitive and symptom-based measures. The effect of AMX0035 on the rate of cognition (ADAS-Cog and MoCA), functioning (DSRS and FAQ), and neuropsychiatric symptoms (NPI-Q) were evaluated. Additionally, effects of AMX0035 were assessed by multi-sequence MRI to evaluate treatment-related changes and consisted of T1 for regional volumetric analyses (especially hippocampus as primary vMRI outcome), T2 FLAIR for assessment of lesions and white matter hyperintensities, and resting state BOLD to measure posterior and anterior default mode network functional connectivity. Safety outcomes, specifically ARIA-E and ARIA-H, were monitored by T2 FLAIR and susceptibility-weighted imaging (SWI).

### 1.2.3 Exploratory Outcome Measures

To assess drug target engagement of PB and TUDCA, a selective panel of CSF biomarkers were analyzed to measure the effects of AMX0035 on pharmacodynamic and pathophysiological targets relevant to AD.

### Biomarker Endpoints

To determine the effects of AMX0035 treatment on:
1. whole brain volume
2. ventricular volume
3. functional MRI measures including connectivity with resting state BOLD;
4. cerebrospinal fluid (CSF) and plasma biochemical markers, including:
   Pathognomonic Alzheimer's analytes: amyloid-B1-42. amyloid-β1-40, total tau, phospho-tau (pTau) 181.
   Markers of neurosynaptic damage
   Markers of metabolism and oxidative stress
   Markers of vascular injury
   Markers of neuroinflammation

Specifically, in CSF, 1) “core” AD biomarkers Aβ₁₋₄₂, Aβ₁₋₄₀, total tau and phospho-tau (pTau) 181; 2) Neurofilament-light (NfL) and neurogranin (Ng) as neuronal injury markers; 3) pyruvate/lactate and 8-hydoxy-2′-deoxyguanosine (8-OHdG) as indicators of mitochondrial function and the redox milieu; and 5) YKL-40, MCP-1, and IL-6 as biomarkers of inflammation in AD were assayed.

Alzheimer's disease is defined by the presence of abundant amyloid plaques and the presence of neurofibrillary tangles in neuronal cortices. These pathologic lesions are composed primarily of Aβ and tau, and extensive studies have now established that CSF measures of these two proteins are sensitive and specific diagnostic markers of AD. Further, these markers may serve as prognostic biomarkers of progression of cognitive decline in MCI.

Neurofilament-light chain is a putative marker of subcortical large-caliber axonal degeneration and has been shown to be elevated in subjects with AD and MCI (Zetterberg, Henrik, et al. “Association of cerebrospinal fluid neurofilament light concentration with Alzheimer disease progression.” JAMA Neurology 73.1 (2016): 60-67). Furthermore, levels of CSF NfL were found to be associated with more rapid AD disease progression. Levels of CSF NfL are thought to be biomarkers of non-specific axonal degeneration and have been demonstrated to be elevated in subjects with inflammatory disease (Constantinescu, Radu, et al. “Cerebrospinal fluid markers of neuronal and glial cell damage in patients with autoimmune neurologic syndromes with and without underlying malignancies.” Journal of Neuroimmunology 306 (2017): 25-30), Creutzfeldt-Jakob disease (van Eijk, Jeroen J J, et al. “CSF neurofilament proteins levels are elevated in sporadic Creutzfeldt-Jakob disease.” Journal of Alzheimer's Disease 21.2 (2010): 569-576; Steinacker, Petra, et al. “Neurofilaments in blood and CSF for diagnosis and prediction of onset in Creutzfeldt-Jakob disease.” Scientific Reports 6 (2016)), progressive supranuclear palsy (Jabbari, Edwin, Henrik Zetterberg, and Huw R. Morris. “Tracking and predicting disease progression in progressive supranuclear palsy: CSF and blood biomarkers.” J Neurol Neurosurg Psychiatry (2017): jnnp-2017), ALS and vascular dementia).

YKL-40 is a secreted glycoprotein considered to be a biological marker of active gliosis and neuroinflammation. CSF YKL-40 levels will therefore be assessed as a biomarker of neuroinflammation. YKL-40 is produced by astrocytes and is significantly elevated in subjects with MCI and mild AD (Craig-Schapiro, Rebecaa, et al. “YKL-40: a novel prognostic fluid biomarker for preclinical Alzheimer's disease.” Biological psychiatry 68.10 (2010): 903-912). However, CSF levels of YKL-40 are non-specific biomarkers of neuroinflammation and have been demonstrated to be elevated in subjects with multiple sclerosis (Comabella, Manuel, et al. “Cerebrospinal fluid chitinase 3-like 1 levels are associated with conversion to multiple sclerosis.” Brain 133.4 (2010): 1082-1093) and traumatic brain injury (Bonneh-Barkay, Dafna, et al. “YKL-40 expression in traumatic brain injury: an initial analysis.” Journal of Neurotrauma 27.7 (2010): 1215-1223). It is not clear whether YKL-40 can be used to measure disease modification with pharmaceutical interventions like AMX0035 because we do not yet have any proven disease modifying drugs for comparison. However, elevated CSF YKL-40 is associated with other promising biomarkers of AD, specifically CSF NfL, T-Tau, and Aβ1-42 (Janelidze, Shorena, et al. “Cerebrospinal fluid neurogranin and YKL-40 as biomarkers of Alzheimer's disease.” Annals of Clinical and Translational Neurology 3.1 (2016): 12-20, and Melab, Kelsey E., et al. “Cerebrospinal fluid markers of Alzheimer's disease pathology and microglia activation are associated with altered white matter microstructure in asymptomatic adults at risk for Alzheimer's disease.” Journal of Alzheimer's Disease 50.3 (2016): 873-886).

Neurogranin (Ng) is a calmodulin-binding post-synaptic protein thought to be expressed exclusively in the brain and particularly enriched in dendritic spines (Hayashi, Yasunori. “Long-term potentiation: two pathways meet at neurogranin.” The EMBO Journal 28.19 (2009): 2859-2860). Ng is hypothesized to play a role in long-term potentiation and memory consolidation (Zhong, Ling, et al. “Increased prefrontal cortex neurogranin enhances plasticity and extinction learning.” Journal of Neuroscience 35.19 (2015): 7503-7508). CSF Ng levels are increased in AD and correlated with levels of “core” AD CSF biomarkers (Janelidze, Shorena, et al. “Cerebrospinal fluid neurogranin and YKL-40 as biomarkers of Alzheimer's disease.” Annals of Clinical and Translational Neurology 3.1 (2016): 12-20, and Portelius, Erik, et al. “Cerebrospinal fluid neurogranin: relation to cognition and neurodegeneration in Alzheimer's disease.” Brain 138.11 (2015): 3373-3385).

Each subject in the study had a CSF sample collected anytime between the Screening Visit and up to 7 days prior to the Baseline Visit and at the Week 24/Early Discontinuation Visit CSF was collected and aliquoted in polypropylene tubes using a standardized protocol and was stored at −80° C. for subsequent analyses.

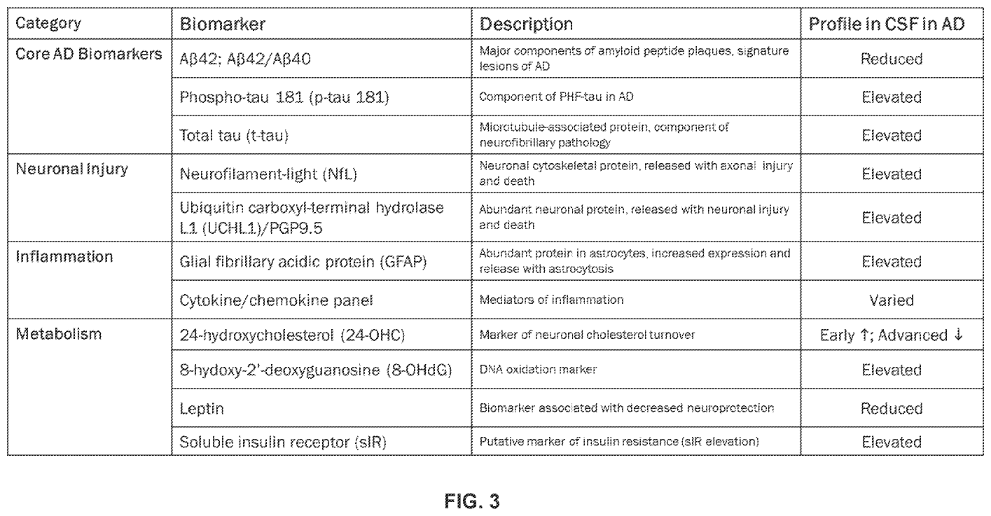

FIG. 3 show the general trend for various biomarkers in AD.

### 2. Study Design

### 2.1 Overall Study Design and Plan

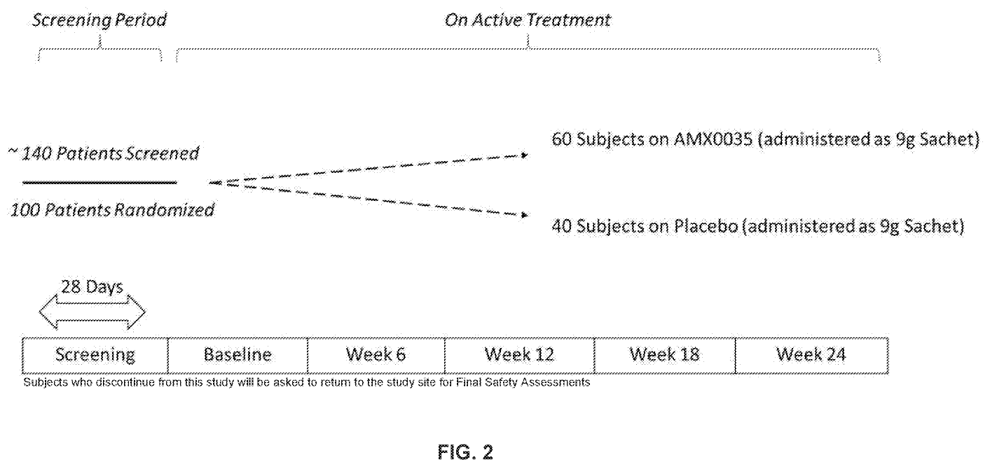

During the enrollment period, approximately 140 subjects were screened and around 100 of those subjects were randomized from approximately 10 AD specialty clinical centers in the US. These subjects were randomly assigned in a 3:2 ratio to oral twice daily sachet of active combination TUDCA/PB or matching placebo. Treatment duration was approximately 24 weeks. Subjects were administered study drug or matching placebo twice daily. Visits occurred at Screening, Baseline, Week 6, Week 12, Week 18, and Week 24. Subjects who dropped out of the study were asked to return for an Early Discontinuation Visit approximately 14 days after last dose of study drug. The overall study workflow is shown in FIG. 2 (note that FIG. 2 is generalized; final randomization was N=95 (n=51 PB/TURSO and n=44 placebo)).

All visit windows were consecutive calendar days and were calculated from the day the participant starts study treatment (Day O is the day of the Baseline Visit and first day of therapy) except the Final Follow-Up Call. The target date for the Final Follow-Up Call was calculated from the last dose of study drug.

### 2.2 Study Duration

Subjects remained on randomized, placebo-controlled, double-blind treatment until the Week 24 Visit. Including the screening and follow-up visits, each subject remained in the study for approximately 8 months. In some cases, the Week 24 visit was delayed due to COVID-19 restrictions, and the study treatment was extended up to Week 40/Day 280. Therefore, in some cases, a subject's participation in the study may have been up to approximately 11 months.

### 3. Study Enrollment and Withdrawal

### 3.1 Inclusion and Exclusion Criteria

### 3.1.1 Inclusion Criteria:

1) Ages 55-89, inclusive, male or female
2) Diagnosed with “Probable Alzheimer's Disease” or “Mild Cognitive Impairment” with a primarily amnestic presentation (deficit in learning and recall of recently learned information) that is accompanied by the presence of documented biomarkers (amyloid PET, CSF AD biomarkers, FDG-PET, or vMRI) supporting that the syndrome is likely due to AD pathology
3) MoCA score≥8
4) Ability to read and write in English sufficiently to complete all study procedures
5) Geriatric Depression Scale <7
6) Willing and able to complete all assessments and study procedures
7) Not pregnant, lactating or of child-bearing potential (women must be >2 years post-menopausal or surgically sterile)
8) Study partner with at least two days per week with contact with the subject willing to accompany patient to visits and complete partner study forms
9) No known hypersensitivity to Tauroursodeoxycholic acid or Phenylbutyrate
10) If on a cholinesterase inhibitor (e.g., donepezil) and/or memantine, treatment must have started for no less than 3 months (84 days) prior to baseline and the dosing regimen must have remained stable for 6 weeks (42 days) prior to baseline. The Investigator anticipates that the dosing regimen at baseline will remain unchanged throughout participation in the study.

### 3.1.2 Exclusion Criteria

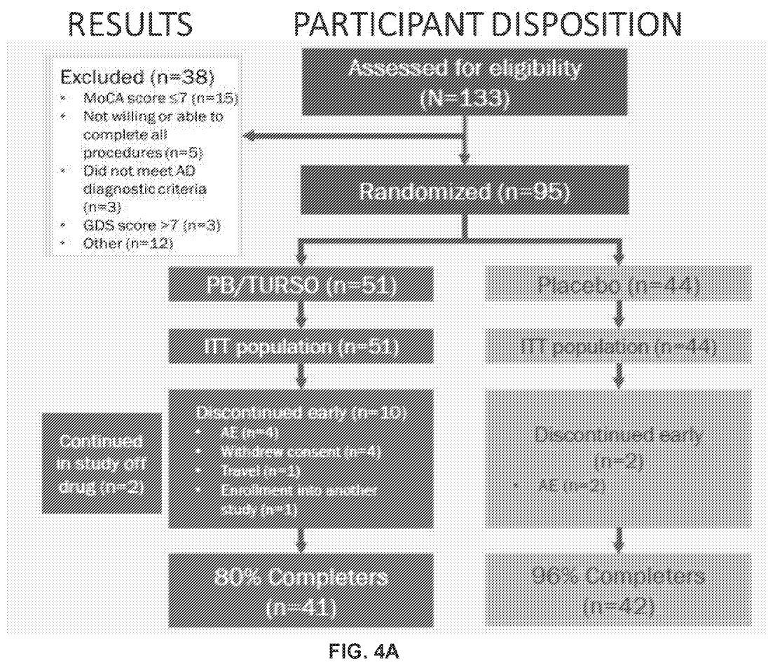

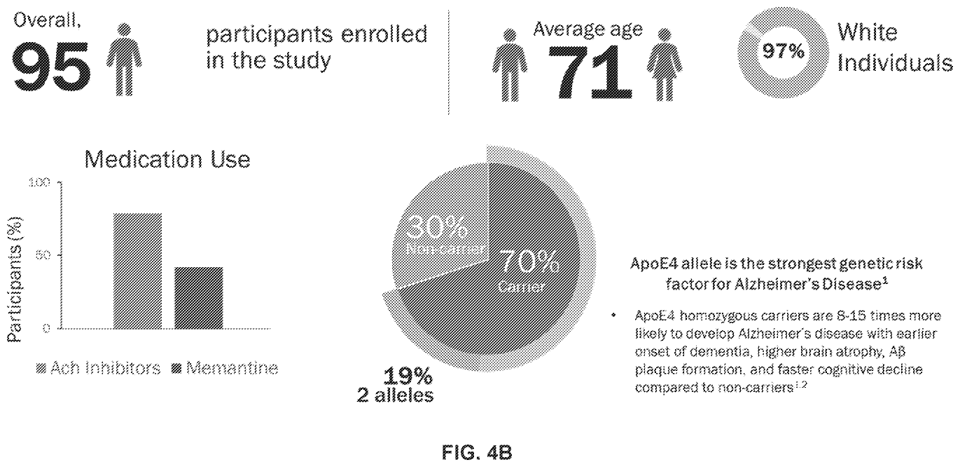

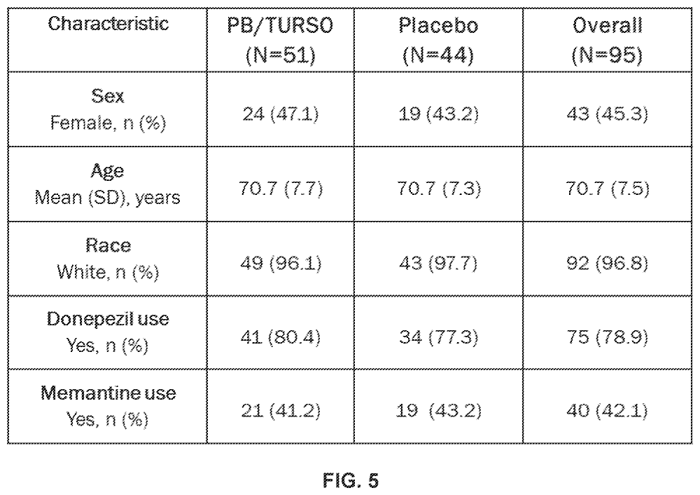

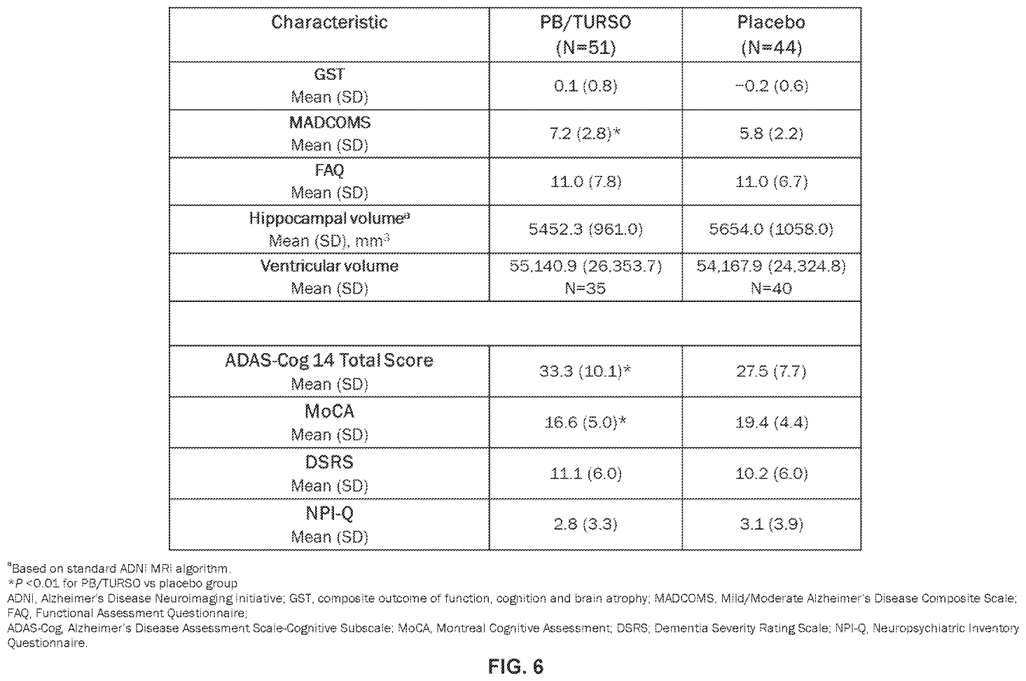

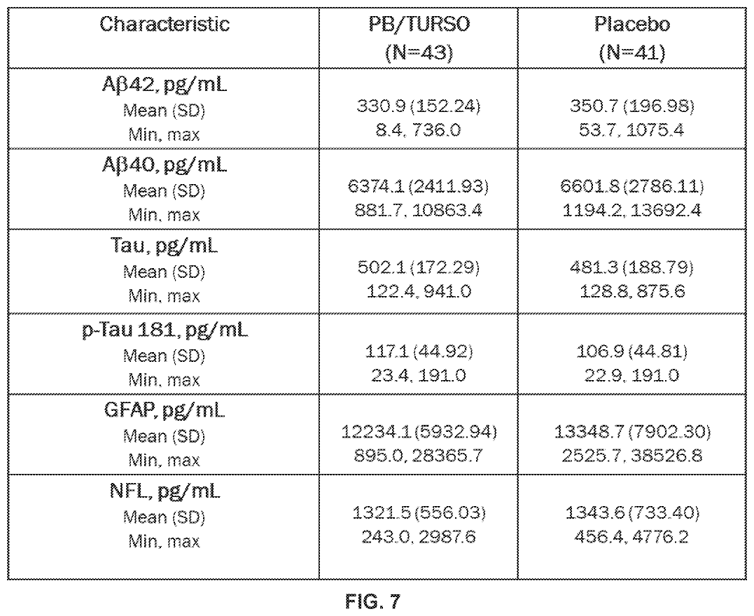

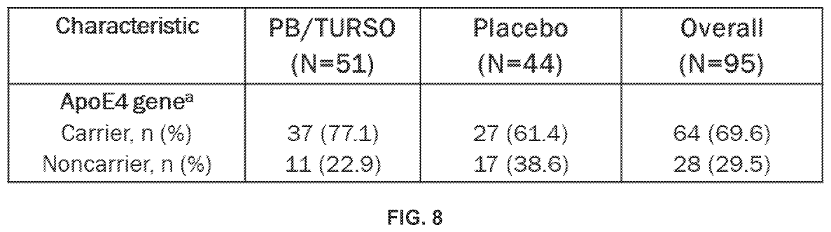

1) Any CNS disease other than suspected AD, such as clinical stroke, brain tumor, normal pressure hydrocephalus, multiple sclerosis, significant head trauma with persistent neurological cognitive deficits or complaints, Parkinson's disease, frontotemporal dementia, or other neurodegenerative diseases
2) Abnormal liver function defined as AST and/or ALT >3 times the upper limit of normal
3) Renal insufficiency as defined by a serum creatinine >1.5 times the upper limit of normal
4) Recent (less than 1 year) cholecystectomy or the presence of post-cholecystectomy syndrome or biliary obstruction
5) Clinically significant unstable medical condition (other than AD) that in the Site Investigator opinion would pose a risk to the participant if they were to participate in the study
6) Any contraindication to undergo MRI studies such as:
   a. History of a cardiac pacemaker or pacemaker wires
   b. Metallic particles in the body
   c. Vascular clips in the head
   d. Prosthetic heart valves
   e. Severe claustrophobia impeding ability to participate in an imaging study, or MRI findings that show one or more of the following:
      a. More than 4 incidental microhemorrhages b. Incidental lacunar infarcts with attributable signs or symptoms and with history of stroke
      b. Incidental meningiomas with attributable signs or symptoms
      c. Newly recognized meningioma
7) Any major active or chronic psychiatric illness (e.g. depression, bipolar disorder, obsessive compulsive disorder, schizophrenia) that is not stable or well controlled within the previous year prior to baseline
8) Any significant neurodevelopmental disability
9) Current suicidal ideation or history of suicide attempt within five years of baseline or significant change from the screening and baseline C-SSRS at the discretion of the Site Investigator
10) History of alcohol or other substance abuse or dependence within the past two years
11) Any significant systemic illness or medical condition that could affect safety or compliance with study at the discretion of the Site Investigator
12) Laboratory abnormalities in B12, TSH, or other common laboratory parameters that might contribute to cognitive dysfunction
13) Current use of medications with psychoactive properties that may deleteriously affect cognition (e.g., anticholinergics, centrally-acting antihistamines, antipsychotics, sedative hypnotics, anxiolytics)
14) Use of any investigational therapy being evaluated for the treatment of AD is prohibited beginning three months (90 days) prior to the Baseline Visit and throughout the study.
15) Use of other investigational agents one month (28 days) prior to the Baseline Visit and for the duration of the trial.
Shown in FIGS. 4-8 and Table 1 are the baseline characteristics of enrolled participants. FIGS. 4A and 4B are schematic representations of the enrolled participants. As seen, in FIG. 5, there were no significant differences between groups in the baseline characteristics. FIG. 6 shows the cognitive scores, as measured by various tests, of the enrolled patients. As seen, the PB/TURSO arm had significantly more cognitively impaired patients but similar functional status (ADAS-Cog14 total score, MOCA, and MADCOMS, all P<0.01). FIG. 7 shows the baseline levels of biomarkers in the CSF. FIG. 8 shows ApoE status (PB/TURSO group had a numerically higher percentage of apolipoprotein E4 carriers compared with the placebo group (77.1% vs 61.4%, respectively; P=0.12).

TABLE 1

| Characteristic | AMX0035 (n = 51*) | Placebo (n = 44*) |
|---|---|---|
| Age, mean (SD) | 70.7 (7.69) | 70.7 (7.30) |
| Male, no. (%) | 27 (52.9) | 25 (56.8) |
| White, no. (%) | 49 (96.1) | 43 (97.7) |
| APOE ε4 carrier, no. (%) | 37 (77.1) | 27 (61.4) |
| 1 allele | 29 (60.4) | 18 (40.9) |
| 2 alleles | 8 (16.7) | 9 (20.5) |
| CSF AB₄₀, mean (SD) pg/mL | 6374.1 (2411.9) | 6601.8 (2786.1) |
| CSF AB₄₂, mean (SD) pg/mL | 330.9 (152.24) | 350.7 (196.9) |
| CSF total tau, mean (SD) pg/mL | 502.1 (172.3) | 481.3 (188.8) |
| CSF p-tau181, mean (SD) pg/mL | 117.1 (44.9) | 106.9 (44.81) |
| CSF NfL, mean (SD) pg/mL | 1321.47 (556.0) | 1343.65 (733.4) |
| CSF GFAP, mean (SD) pg/mL | 12234.1 (5932.9) | 13348.7 (7902.3) |
| CSF Neurogranin, mean (SD) | 466.2 (176.7) | 446.0 (206.2) |
| pg/mL | | |
| CSF AB 1-42/1-40 ratio mean | 0.05(0.02) | 0.05 (0.02) |
| (SD) pg/mL | | |
| CSF YKL-40 mean (SD) pg/mL | 223932.2 (76102.7) | 229273.6 (80806.0) |
| CSF IL-6 mean (SD) pg/mL | 1228.2 (610.2) | 1163.5(423) |
| CSF IL-8 mean (SD) pg/mL | 26.1 (6.8) | 24.73 (5.8) |
| CSF IL-15 mean (SD) pg/mL | 3.3 (1.0) | 3.0 (1.0) |
| CSF MCP-1 mean (SD) pg/mL | 246.4 (75.7) | 242.6(64.6) |
| CSF FABP3 mean (SD) pg/mL | 4059.3 (1445.1) | 3926.4 (1517.5) |
| CSF 8-OHdG mean (SD) pg/mL | 3.1 (0.7) | 3.24 (1.0) |
| CSF 24-OHC mean (SD) pg/mL | 7.38 (2.2) | 7.30 (2.7) |
| CSF MMP-10 mean (SD) pg/mL | 65.5 (35.0) | 52.50(20.2) |
| CSF leptin mean (SD) pg/mL | 124.9 (84.6) | 102.81 (61.3) |
| CSF sIR mean (SD) pg/mL | 4.4 (1.4) | 4.47 (1.3) |

Abbreviations: AB, amyloid beta:: APOE ε4, apolipoprotein E. gene ε4 allele: CSF, cerebrospinal fluid: FABP3, fatty acid binding protein [symbol] GFAP, glial [symbol] acidic protein: IL, interleukin; MCP-1; Monocyte chemoattractant protein-1:MMP-10, matrix metailoproteinase 10.NfL, neurofilament light chain: PB and TURSO: sodium phenylbutyrate and tauroursodiol; p-tau, phosphorylated tau: SD, standard deviation; sIR, soluble insulin receptor-8-OHdG.B-hydroxy-2- [symbol] 24-OHC, 24S-hydroxycholesterol [symbol]
indicates data missing or illegible when filed

### 3.2 Treatment Assignment Procedure

Each subject who met all eligibility criteria was randomized to receive either therapy by twice daily sachet of AMX0035 (3 g PB and 1 g TUDCA) or matching placebo for 24 weeks of treatment. For the first 1 week of the study, subjects only took a single sachet daily and were instructed to increase to 2 sachets daily at the Week 1 Visit.

### 3.3 Reasons for Withdrawal

A study subject was discontinued from participation in the study if:
Any clinical adverse event (AE), laboratory abnormality, concurrent illness, or other medical condition or situation occurred such that continued participation in the study was not in the best interest of the subject.
The subject met any exclusion criteria (either newly developed or not previously recognized).

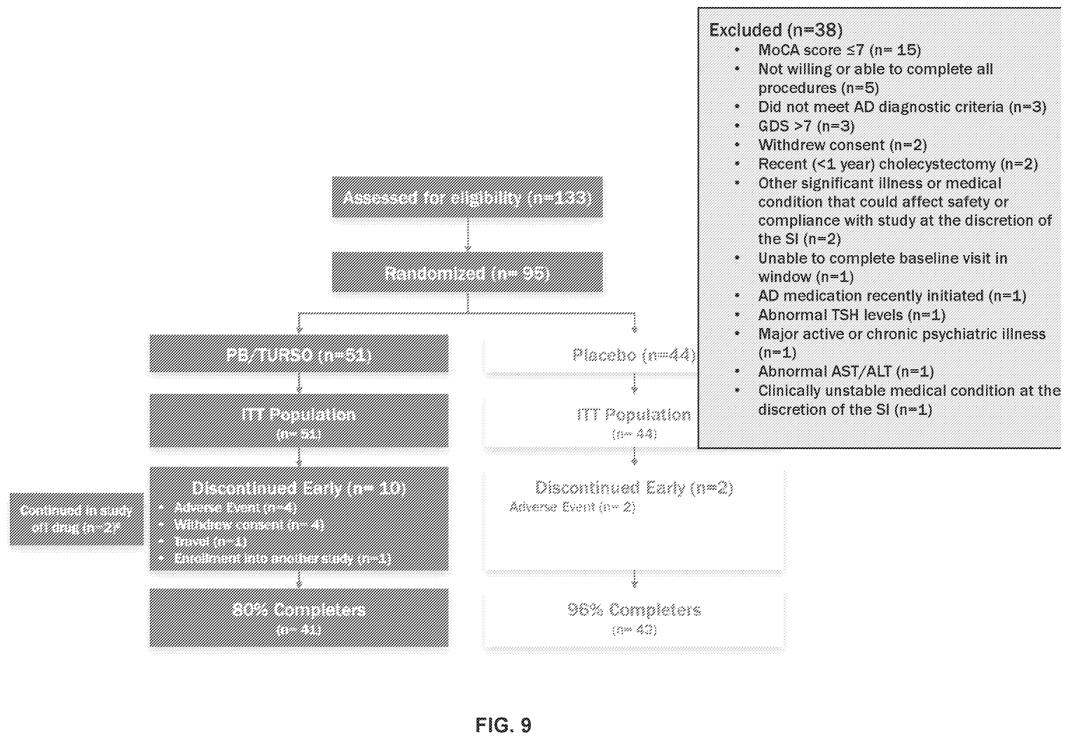

Subjects were free to withdraw from participation in the study at any time upon request. A summary of those that were excluded, withdrawn, or discontinued early is shown in FIG. 9.

### 4. Treatments Administered

### 4.1 Treatments

### 4.1.1 Study Product Description

AMX0035 is a combination therapy comprised of two active pharmaceutical ingredients, sodium phenylbutyrate (PB and tauroursodeoxycholic acid (TUDCA).

Phenylbutyrate is an approved compound in the United States for urea cycle disorders and is marketed in the US as Buphenyl®. There is an existing USP monograph for this material. The drug substance PB is produced cGMP conditions. The manufacture and controls for PBA are described in Drug Master File No. 019569. The specifications for PB are identical to those of the Ph. Eur. The chemical structure for PB is provided below.

### Chemical Structure of PB:

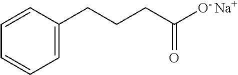

The drug substance TUDCA is currently marketed under the brand name Tudcabil® and Taurolite®. It is used for the indications of treatment of cholesterol gallstones. It has been used for the treatment of cholestatic liver diseases including primary cirrhosis, pediatric familial intrahepatic cholestasis, primary sclerosing cholangitis, and cholestasis due to cystic fibrosis. The chemical structure for TUDCA is provided below.

### Chemical Structure TUDCA:

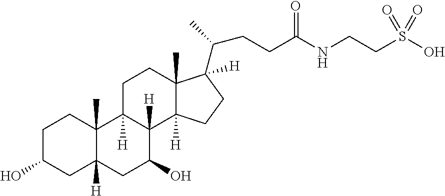

A powder filled sachet was used as the AMX0035 drug product. The drug product was filled under cGMP conditions in an aluminum foil lined sachet.

The sachet will contain 2 active study drug ingredients and excipients.

### 4.1.2 Placebo

A matched placebo was used to maintain the dosage-blind. The placebo sachets for this study matched the corresponding AMX0035 sachets in size, color, and presentation Administration of matching placebo was the same as for subjects in the treatment group.

### 4.2 Product Storage and Stability

All investigational drug supplies were kept at ambient temperature 15-25° C. Subjects were asked to store the kits containing the sachets away from moisture at room temperature. Stability has been assessed both at ICH standard and accelerated conditions for each of the individual active ingredients and they were found to be stable over five years. Drug product received regular stability testing over the course of the study to ensure product did not degrade.

### 4.3 Dosage, Preparation and Administration of Study Intervention/Investigational Product

Subjects were instructed to open the sachet of AMX0035 and add it to a cup or other container (see detailed “Instructions to Subjects” below). Subjects were then instructed to add approximately 8 oz. (1 cup) of room temperature water, stir until the powder is mostly dissolved, and consume the drink completely. It is normal for a small amount of powder to remain undissolved. Subjects were instructed to consume within one hour after the powder is added to water. Subjects may consume other beverages after consumption of study drug; however, subjects should not mix study drug with any liquid other than water.

### Instructions to Subjects

The following instructions was provided orally to the patient at the Baseline Visit by a healthcare staff member. Please have the Listerine® products (Pocketpaks® and Pocketmist®) available for demonstration.
Alert the patient that the study drug has a bitter taste, but that there are ways to make it more palatable (see below).
Rip open the sachet of study drug and add it to a cup or other container and add approximately 8 oz. (1 cup) of room temperature water and stir vigorously. Study drug may require significant stirring or gentle crushing to dissolve.
The treatment should be taken within one hour of mixing into water.
Several things may be done to reduce the bad taste and make the drug more palatable:
   Use Listerine Pocket Packs® (strips) or a Listerine PocketMist® (spray) immediately before and/or immediately after taking the drug. Use liberally to coat the mouth. This has been found to significantly mask the bitter taste.
   Take a snack or a meal after taking your treatment.
   Follow drug immediately with milk to remove taste from the mouth.
   Avoid drinking fruit juice at the same time as the drug as this may make flavor worse.
   Mixing the study drug with a liquid other than water should be avoided.

### 4.3.1 Feeding Tube Study Drug Administration

For subjects with a gastrostomy or nasogastric (feeding) tube, the study drug may be dissolved in water as per the procedures outlined in the section “Treatment Assignment Procedures” and the study drug may be administered via the feeding tube.

### 4.4 Modification of Study Intervention/Investigational Product for a Subject

### 4.4.1 Dosage Discontinuation

Reasons for discontinuation of study medication may include an AE, Medical Monitor or SI recommendation, sponsor termination, protocol deviation, loss-to-follow-up, subject request, or death. All serious adverse events (SAEs) that occur in a subject who has discontinued early must be recorded and reported within 24-hours of awareness.

Study subjects who discontinued the study drug prematurely (early termination from study) and decided to not remain in the intention-to-treat (ITT) portion of the study were encouraged to return for a Final Safety/Early Termination Visit and participate in a Follow-Up Telephone Call as outlined in the section “Handling of Withdrawals”. All subjects who discontinued study drug early and chose to remain in the ITT portion of the study were encouraged to follow the study visits, off drug, up to the time of the Final Follow-up Telephone Call.

### 4.5 Prior and Concomitant Therapy

Any small molecule investigational therapy being used or evaluated for the treatment of AD is prohibited beginning 3 months (84 days) prior to the Baseline Visit and throughout the study. Any immunotherapy investigational therapy is prohibited beginning 1 year (365 days) prior to the Baseline Visit and throughout the study.

### 4.5.1 Prohibited Medications and Contraindications

### Prohibited Medications

Prohibited medications include but are not limited to:
HDAC Inhibitors including:
   Valproate
   Vorinostat (Zolinza)
   Romidepsin
   Chidamide
   Panobinostat
   Lithium
   Butyrate
   Suramin
Probenecid
Bile Acid Sequestrants including:
   Cholestyramine and Cholestyramine Light
   Questran and Questran Light
   Welchol
   Colestid and Colestid Flavored
   Prevalite

### Antacids:

Antacids containing Aluminum hydroxide or smectite (aluminum oxide) may not be taken within two hours of administration of AMX0035 as they inhibit absorption of TUDCA. These include:
Alamag
Alumina and Magnesia
Antacid, Antacid M and Antacid Suspension
Gen-Alox
Kudrox
M.A.H.
Maalox HRF and Maalox TC
Magnalox
Madroxal
Mylanta and Mylanta Ultimate
Ri-Mox
Rulox
   Aricept, Exelon, Razadyne, Namenda—If on any of these drugs, treatment must have started for no less than 3 months (84 days) prior to baseline and the dosing regimen must have remained stable for 6 weeks (42 days) prior to baseline.

### 5. Study Schedule

### 5.1 Screening Visit

The following procedures were performed at an office visit to determine the subject's eligibility for the study and the completion of assessments and parameters will take approximately 3-4 hours:
Obtain written informed consent from subject
Assess inclusion and exclusion criteria to determine subject eligibility
Obtain medical history and demographics
Review and document concomitant medications and therapies
Obtain AD diagnosis history, medical history, and demographic information
Assess and document adverse events (AEs) after subject signs informed consent form (ICF)
Measure vital signs (blood pressure, heart and breathing rates, temperature) including height and weight
Perform physical and neurological examinations
Perform 12-lead ECG (Electrocardiogram)
Administer Montreal Cognitive Assessment (MoCA)
Administer the C-SSRS Screening Version questionnaire
Administer Geriatric Depression Scale
Administer Neuropsychiatric Inventory Questionnaire (NPI-Q)
Perform MRI Scan (to be completed anytime between the Screening and up to 7 days prior to the Baseline Visit)
Collect blood samples for
   Clinical laboratory assessments including Hematology (CBC with differential),
   Complete Chemistry Panel, Liver Function Tests
Collect urine sample for urinalysis
Perform lumbar puncture to collect CSF for biomarker analysis (to be completed anytime between the Screening Visit and up to 7 days prior to the Baseline Visit, pre-dose)
Schedule the Baseline Visit

### 5.1.1 Screen Failures

Any subject who signed consent was considered enrolled in the study. If a subject failed screening, at a minimum, the following information was captured and entered in the Electronic Data Capture (EDC) System:
Inclusion/Exclusion Criteria
Demographics
Reason for screen failure

### 5.2 Baseline Visit

This visit took place no more than 28+5 days after the Screening Visit. The following procedures were performed and took approximately 3-4 hours:
Confirm eligibility criteria are still met
Randomize subject
Review and document concomitant medications and therapies
Review and document Adverse Events
Measure vital signs (blood pressure, heart and breathing rates, temperature)
Administer DSRS
Administer FAQ
Administer Neuropsychiatric Inventory Questionnaire (NPI-Q) if not performed at screening
Administer ADAS-Cog
Administer the C-SSRS Since Last Visit questionnaire
Collect blood samples for:
   Biomarkers
   Genetic analysis

Administer first dose of study drug. The healthcare staff member will advise the subject on appropriate delivery.
Dispense 6 weeks of study drug

### 5.3 Week 1 Phone Call

This visit took place 7±1 day after the Baseline Visit. The following procedures were performed and took approximately 15 minutes:
Review and document Adverse Events
Ask about study drug compliance and accountability
Notify subjects of increase from one sachet per day to two sachets per day

### 5.4 Week 6 Visit

This visit took place 42±14 days after the Baseline Visit. The following procedures were performed and took approximately 30 minutes-1 hour. To the extent possible, the Week 6 visit was done remotely. If completed remotely, unused study drug was collected, and compliance determined, at the next in-person visit. If allowed by local regulations and local IRB this visit was performed at the subject's residence or preferred location:
Review and document concomitant medications and therapies
Review and document Adverse Events
Administer MoCA
Administer the C-SSRS questionnaire (Since Last Visit)
Ask about study drug compliance and accountability
Collect any unused study drug (optional empty study drug sachets)
Dispense 6 weeks of study drug
Schedule next study visit

### 5.5 Week 12 Visit

This visit took place 84±28 days after the Baseline Visit.

It was preferred that the Week 12 visit be conducted at the site with the participant physically present. If it was not possible to complete an in-person Week 12 visit at the site, the safety assessments below were completed by Week 16/Day 112 for the subject to remain on study drug. Sites were allowed to make alternative arrangements to complete these assessments per institutional and IRB policy.
ECG
Safety Labs: Hematology (Complete Blood Count with Differential), Complete Chemistry Panel, Liver Function Tests, and Urinalysis
Vital Signs
C-SSRS

If completed remotely, unused study drug was collected, and compliance determined, at the next in-person visit.

The following procedures were performed and took approximately 2-3 hours:
Measure vital signs (blood pressure, heart and breathing rates, temperature)
Review and document concomitant medications and therapies
Review and document Adverse Events
Ask about study drug compliance and accountability
Administer ADAS-Cog
Administer DSRS
Administer FAQ
Administer Neuropsychiatric Inventory Questionnaire (NPI-Q)
Administer the C-SSRS questionnaire (Since Last Visit)
Perform a 12-Lead ECG
Collect blood for:
   Routine safety lab tests (chemistry and hematology assessment)
   Biomarkers
   Pharmacokinetics (PK)
Collect urine sample for urinalysis
Collect any unused study drug (optional empty study drug sachets)
Dispense 6 weeks of study drug
Schedule next study visit

### 5.6 Week 18 Visit

This visit took place 126±14 days after the Baseline Visit. The following procedures were performed and took approximately 30 minutes-1 hour. To the extent possible, the Week 18 visit was done remotely. If completed remotely, unused study drug was collected, and compliance determined, at the next in-person visit. If allowed by local regulations and local IRB this visit was performed at the subject's residence or preferred location:
Review and document concomitant medications and therapies
Review and document Adverse Events
Administer MoCA
Administer the C-SSRS questionnaire (Since Last Visit)
Collect any unused study drug (optional empty study drug sachets)
Ask about study drug compliance and accountability
Dispense 6 weeks of study drug
Schedule next study visit

### 5.7 Week 24 Final Study Visit/Early Discontinuation Visit

This visit took place 168±28 days after the Baseline Visit.

All reasonable efforts were made to complete the Week 24 or Early Discontinuation visit at the site with the participant physically present. However, if COVID-19 related restrictions (e.g., site closure, travel restrictions) made it impossible to conduct an in-person clinic visit during the specified window, then any assessment that could be performed remotely was completed as an unscheduled visit during the Week 24 window. The actual Week 24 visit, with all of the assessments indicated on the SOA, could be postponed for up to 12 weeks and treatment extended. The maximum duration a subject could be on IP is 40 weeks. Safety checks (i.e., ECG, safety labs, vital signs, and C-SSRS) had to be completed, at minimum, every 16 weeks/112 days. If safety assessments were performed so that the Week 24 visit was postponed, the assessments were documented as an unscheduled visit.

If possible, an Early Discontinuation visit was done within 14 days of the last dose of IP (i.e., last dose of IP+14 days). The MRI and lumbar puncture was done as soon as was practical and, if possible, within 60 days of the last dose of IP (i.e., last dose of IP+60 days). The following procedures were performed and took 3-4 hours:
Measure vital signs (blood pressure, heart and breathing rates, temperature) including weight
Review and document concomitant medications and therapies
Review and document Adverse Events
Perform Physical and neurological exam
Ask about study drug compliance and accountability
Administer ADAS-Cog
Administer DSRS
Administer FAQ
Administer Neuropsychiatric Inventory Questionnaire (NPI-Q)
Administer MoCA
Perform MRI scan
Perform lumbar puncture to collect CSF for biomarker analysis (can be collected up to 7 days prior to this visit. If the early discontinuation visit occurs up to 7 days after initiation of study drug, then the LP does not need to be completed)
Administer the C-SSRS questionnaire (Since Last Visit)
Perform a 12-Lead ECG
Collect blood for:
   Routine safety lab tests (chemistry and hematology assessment)
   Biomarkers
   PK (will not be done for subjects completing an Early Discontinuation Visit)
Collect urine sample for urinalysis
Collect any unused study drug (optional empty study drug sachets)
Ask about study drug compliance and accountability
Schedule final follow-up telephone call as needed

### 5.7.1 Final Follow-Up Telephone Call

A follow-up phone call took place after the Week 24 visit and within 14±5 days after the subject's last dose of study drug. For participants who discontinued treatment early, the Final Follow-Up Call was not required if the Early Discontinuation visit occurs 14±5 days after administration of the last dose of study drug.

The following were performed and took approximately 15 minutes:
Review and document concomitant medications and therapies
Assess and document AEs, including AEs specified in 9.2.3

### 5.8 Protocol Deviations

A protocol deviation was any noncompliance with the clinical trial protocol, Good Clinical Practice (GCP). The noncompliance could either be on the part of the subject, the SI, or the study site staff. In the event of deviations, corrective actions were to be developed by the site in conjunction with the coordinating team and implemented promptly.

All deviations from the protocol were addressed in the subject's source documents. Protocol deviations were sent to the local IRB per their guidelines and entered in the Protocol Deviations Log in the EDC system.

### 5.9 Missed Visits and Procedures

Missed visits and any procedures not performed (not attempted) for reasons other than illness, injury or progressive disability (i.e. subject was physically unable to perform test) were reported as protocol deviations Multiple missed study procedures, due to COVID-19, that did not affect data integrity or patient safety, were documented and combined into one (1) minor protocol deviation

Procedures or visits not performed due to illness, injury or disability, including procedures that were attempted but failed (i.e. blood samples unable to be drawn after multiple attempts, or weight unable to be obtained due to subject immobility) were not reported as protocol deviations.

Study drug compliance that was outside the limits set in the study operations manual were reported as a protocol deviation.

Details and specific instructions regarding protocol deviations, including any exceptions to this standard procedure, are found in the study operations manual.

### 6. Clinical Assessments and Outcome Measures

### 6.1 Clinical Variables

Assessments were performed at designated time-points throughout the study for clinical evaluation. In addition to the assessments evaluated below, subjects provided information on their demographics, medical and AD diagnostic history, and concomitant medication usage.

### 6.1.1 Vital Signs, Height, & Weight

Vital signs were obtained after the subject has been in a seated position for several minutes. Vital signs, including blood pressure, heart rate, respiratory rate and body temperature were assessed at Screening, Baseline, Week 12, and Week 24/Early Discontinuation Visit. Height will only be collected once at the Screening Visit. Weight was collected when a physical exam is done at the Screening and Week 24/Early Discontinuation Visit.

If the subject cannot attend a visit at the site to have vitals measured because of COVID-19, other arrangements may be made to measure vitals as allowed per institution and local IRB policy.

### 6.1.2 Clinical Laboratory Assessments

The following laboratory tests were performed for safety purposes at Screening, Week 12, and Week 24/Early Discontinuation Visits:
Hematology with differential panel: complete blood count with differential (hematocrit, hemoglobin, platelet count, RBC indices, total RBC, Total WBC, and WBC & differential).
Blood chemistry panel/liver function tests (LFTs): alanine aminotransferase (ALT (SGPT)), aspartate aminotransferase (AST (SGOT)), albumin, alkaline phosphatase (ALP), bicarbonate, blood urea nitrogen, calcium, chloride, creatinine, glucose, magnesium, phosphate, potassium, sodium, total bilirubin and total protein.
Urinalysis: albumin, appearance, bilirubin, blood, color, glucose, ketones, nitrate, pH, protein, specific gravity, urobilinogen and WBC screen.
B12 and TSH were assessed at Screening Visit only as common laboratory assessments that may contribute to cognitive dysfunction.

All subjects will have had safety laboratory tests at the designated visits outlined in the protocol. These samples were analyzed at a central laboratory. The SI may order additional testing, if thought to be necessary, to further assess an adverse event (AE) or if there is any suspicion that a subject may be pregnant, throughout the course of the study.

If the subject cannot attend a visit at the site to have samples collected because of COVID-19, other arrangements may be made for sample collection and analysis as allowed per institution and local IRB policy (e.g., sample may be analyzed by site or third-party laboratory).

### 6.1.3 Blood Draw and Urine Sample

Subjects will provide additional blood samples at baseline for genetic analysis and at Baseline, Week 12, and Week 24/Early Discontinuation Visits for biomarkers. All samples were anonymized and labeled with a code that will not include any identifiable information. The samples are for research purposes only. Although genetic information may be analyzed, no genetic information was given to the subject.

There was no scheduled date by which the samples were destroyed. Samples were stored for research until they were used, damaged, decayed, or otherwise unfit for analysis. Subjects had the option of declining participation in this portion of the study at any time by withdrawing their consent to have their sample used. However, it was not possible to destroy samples that may have already been used.

Additionally, the central laboratory stored ship blood samples for pharmacokinetic analysis. The central laboratory facility prepared kits for every site detailing the sampling protocol.

### 6.1.4 12-Lead Electrocardiogram (ECG)

A standard 12-lead ECG was performed at Screening, Week 12, and Week 24/Early Discontinuation Visits. Tracings was reviewed by a central ECG reader and a copy of the tracings was kept at clinic sites as part of source documentation. The central ECG reader provided standard ECG devices to each site as well as training. Board-certified cardiologists from the central ECG reader reviewed all ECGs.

If the subject could not attend a visit at the site to have an ECG performed because of COVID-19, arrangements were made to complete the ECG elsewhere using a device not provided by the central reader as allowed per institution and local IRB policy.

### 6.1.5 Physical and Neurological Examination

A physical and neurological examination was performed at Screening and Week 24/Early Discontinuation Visits. The following systems were examined: general appearance, head, eyes, ears, nose, throat, neck, chest, heart, abdomen, extremities, edema, peripheral vascular, skin and appendages, musculoskeletal, central nervous system and back.

### 6.1.6 Geriatric Depression Scale (GDS)

The Geriatric Depression Scale (Short Form) was performed at the Screening Visit only (see below for the scale). The GDS is a questionnaire designed to identify and quantify the presence of depression in the elderly. The scale consists of 15 yes/no questions related to how the subject has felt over the previous week. The GDS includes items to which positive and negative answers were indicative of a symptom of depression. One point was given for each such appropriate answer, with a possible total of 15 points. Total scores of 0-5 were considered normal and scores of 6-15 were considered indicative of depression. A GDS less than 7 was required for inclusion in the study.

### Geriatric Depression Scale

Choose the best answer for how you have felt over the past week:
1. Are you basically satisfied with your life? YES/NO
2. Have you dropped many of your activities and interests? YES/NO
3. Do you feel that your life is empty? YES/NO
4. Do you often get bored? YES/NO
5. Are you in good spirits most of the time? YES/NO
6. Are you afraid that something bad is going to happen to you? YES/NO
7. Do you feel happy most of the time? YES/NO
8. Do you often feel helpless? YES/NO
9. Do you prefer to stay at home, rather than going out and doing new things? YES/NO
10. Do you feel you have more problems with memory than most? YES/NO
11. Do you think it is wonderful to be alive now? YES/NO
12. Do you feel pretty worthless the way you are now? YES/NO
13. Do you feel full of energy? YES/NO
14. Do you feel that your situation is hopeless? YES/NO
15. Do you think that most people are better off than you are? YES/NO

Answers in bold indicate depression. Score 1 point for each bolded answer.
A score >5 points is suggestive of depression.
A score ≥10 points is almost always indicative of depression.
A score >5 points should warrant a follow-up comprehensive assessment.
   Source: http://www.stanford.edu/˜vesavage/GDS.html

This scale is in the public domain.

The Hartford Institute for Geriatric Nursing would like to acknowledge the original author of this Try This, Lenore Kurlowicz, PhD, RN, CS, FAAN, who made significant contributions to the field of geropsychiatric nursing and passed away in 2007.

### 6.1.7 Columbia Suicide Severity Rating Scale (C-SSRS)

The US FDA recommends the use of a suicidality assessment instrument that maps to the Columbia Classification Algorithm for Suicide Assessment (C-CASA) 40. The C-CASA was developed to assist in coding suicidality data accumulated during the conduct of clinical trials of antidepressant drugs. The Columbia Suicide Severity Rating Scale (C-SSRS) 23 was utilized in this trial. The C-SSRS involved a series of questions designed to detect possible suicidal thinking and behavior.

At the Screening Visit, the C-SSRS Screening Version was administered. This version was used to assess suicidality over a specified time-period. The Since Last Visit Version of the C-SSRS was administered at Baseline, Week 6, Week 12, Week 18 and Week 24/Early Discontinuation Visits. This version of the scale assessed suicidality since the subject's last visit.

### 6.1.8 Adverse Events

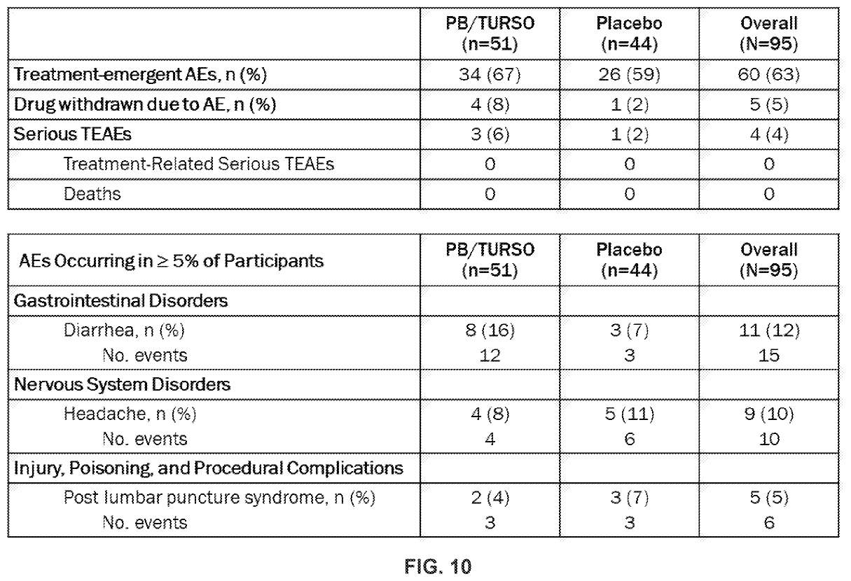

Adverse events (AEs) were documented at each study visit, including the Screening Visit once the informed consent form had been signed by the subject, and at all study visits, including the Final Follow-up Telephone Call 14 days (+5 days) after the last dose of study drug. Information on adverse effects of study medication and on inter-current events were determined at each visit by direct questioning of the subjects, review of concomitant medications, and vital sign results. FIG. 10 shows the summary of adverse events in the intent-to-treat population. As shown, there are no new safety concerns. The PB/TURSO arm had a significantly higher percentage of discontinuations vs. placebo (20% [10/51] vs. 5% [2/44]) and reduced drug compliance vs. placebo (71% vs. 93%).

### 6.2 Outcome Measures

### Global Statistical Test

A GST allows assessment of a global change in disease status/trajectory by standardizing and then combining measures. The GST was a combination of 3 change from baseline to end of study endpoints (MADCOMS, FAQ, and total hippocampal brain volume). The GST was calculated for each subject as a mean score across the three component endpoints. This mean score was analyzed as the primary efficacy outcome variable.

### MADCOMS

ADAS-Cog 14 is not specifically targeted to the mild/moderate stage of AD. A mild/moderate AD composite scale (MADCOMS) was previously optimized for the two distinct groups, mild AD (baseline MMSE 20-26) and moderate AD (baseline MMSE 14-19). The weighted composite was derived using PLS regression from ADAS-Cog, MMSE, and CDR individual items (S.Hendrix ADPD 2021).

$$Moderate MADCOM = Comprehension * 0.36390157 + Word Finding * 0.10931155 + Ideational Praxis * 0.42535667 + Naming Objects * 0.65626894 + Word Recognition * 0.05159097 + Word Recall * 1.0698506 + Spoken Language * 0.3019936 + Home and Hobbies * 0.66529282 + Memory * 0.12277257 - Orientation to Place * 0.23001218 - Spell Backward * 0.07980965 - Language and Praxis * 0.18954955$$

$$Mild MADCOM = Word Finding * 0.39065568 + Word Recall * 1.14084544 + Spoken Language * 1.0989559 + Personal Care * 0.60865765 + Community Affairs * 0.15706995 + Judgment * 1.40920029 - Orientation to Time * 0.27596627$$

The current study collected DSRS (equivalent to CDR) and MoCA (equivalent to MMSE). The equivalent DSRS and MoCA test items were scaled to the MMSE or CDR test range and used to make MADCOMS.

### MMSE to MoCA Conversion:

| | MoCA | |
|---|---|---|
| MMSE Domain | question(s) | scale |
| Orientation to Time | 17-20 | 5/4 |
| Orientation to Place | 21-22 | 5/2 |
| Attention and | 10 | 5/3 |
| Calculation | | |
| Recall | 14 | 3/5 |
| Naming | 6 | 2/3 |
| Repetition | 11 | 1/2 |
| Commands | 9 | 3/1 |
| Reading | 13 | 1/2 |
| Drawing | 2 | 1 |

### CDR to DSRS Conversion:

| | DSRS |
|---|---|
| CDR Domain | question(s) |
| Memory | 1 |
| Orientation | 4-5 |
| Judgement and | 6 |
| Problem Solving | |
| Community Affairs | 7 |
| Home and Hobbies | 8 |
| Personal Care | 9 & 11 |

DSRS values were converted to CDR scales by dividing the sum of the DSRS questions that make up the equivalent CDR domain score by the max score for those questions. Memory, orientation, judgement and problem solving, community affairs, as well as home and hobbies are multiplied by 4 to create 5 categories. That are assigned to 0, 0.5, 1, 2, or 3 by their rank value. Personal care scores are multiplied by 3 to create 4 categories that are assigned to 0, 1, 2, or 3 by their rank value.

### 6.2.1 Functional Activities Questionnaire (FAQ)

The FAQ is a brief informant-administered rating scale used to determine a subjects' level of functional independence when performing a range of instrumental activities of daily living (IADLs), with repeat assessments useful for monitoring performance in these areas over time⁴⁷ (see below for the scale). The FAQ total score (ranging from 0-30) reflects the sum of ordinal ratings (0=fully independent, 1=has difficulty but does by self, 2=requires assistance, and 3=dependent) across ten items assessing a variety of functional activities (i.e., preparing a balanced meal, financial management skills, and shopping), with higher scores indicating increasing levels of dependence. For activities not normally undertaken by a person, a score of 1 was assigned if the informant believed the subject would be unable to complete the task if required, or a score of 0 was assigned if the informant believed the subject could successfully carry out the task if needed. Overall, the FAQ is a sensitive marker of functional impairment among individuals with varying dementia severity⁴³, and has been shown to differentiate mild cognitive impairment from early Alzheimer's Disease with 80% sensitivity and 87% specificity⁴⁹. The FAQ demonstrates high reliability (exceeding 0.90), takes about 5 minutes to complete, and requires limited rater training to administer⁴⁷. The FAQ was administered at the Baseline, Week 12, and Week 24/Early Discontinuation Visits.

### Functional Activities Questionnaire

### Administration

Ask informant to rate patient's ability using the following scoring system:
Dependent=3
Requires assistance=2
Has difficulty but does by self=1
Normal=0
Never did [the activity] but could do now=0
Never did and would have difficulty now=1
Writing checks, paying bills, balancing checkbook
Assembling tax records, business affairs, or papers
Shopping alone for clothes, household necessities, or groceries
Playing a game of skill, working on a hobby
Heating water, making a cup of coffee, turning off stove after use
Preparing a balanced meal
Keeping track of current events
Paying attention to, understanding, discussing TV, book, magazine
Remembering appointments, family occasions, holidays, medications
Traveling out of neighborhood, driving, arranging to take buses
TOTAL SCORE:

### Evaluation

Sum scores (range 0-30). Cutpoint of 9 (dependent in 3 or more activities) is recommended to indicate impaired function and possible cognitive impairment.

Pfeffer R I et al. Measurement of functional activities in older adults in the community. J Gerontol 1982; 37 (3): 323-329.

### 6.2.2 Dementia Severity Rating Scale (DSRS)

The DSRS is a brief 12-item questionnaire administered to an informant that assessed a subjects' functional abilities⁴⁴ and offered a global characterization of everyday activities that were impacted by neurodegenerative disease (see below for the questionnaire). The DSRS is designed in a multi-choice format with strong concurrent validity and parallel content to material covered on the Clinical Dementia Rating Scale (CDR), a commonly employed dementia staging instrument⁴⁶. The DSRS is a highly reliable scale with an intra-class correlation of >90% for interrater reliability and Cronbach's alpha >0.70 for internal consistency⁴⁸, and has been shown to accurately discriminate between cognitive healthy individuals and dementia subjects of varying severity^{44,45}. Further, the DSRS allows for a broad range of scores (total score 0-54) making it suitable to quantify a wide range of functional impairment without being hampered by floor effects seen in more advanced disease, while also making it sensitive to detecting incremental change in functional ability over time⁵⁰. The DSRS takes about 5 minutes to administer, requires minimal rater training, and can be administered over the phone to study subjects if required. The DSRS was administered at the Baseline, Week 12, and Week 24/Early Discontinuation Visits.
PARTICIPANT'S NAME: ______ DATE: ______
PERSON COMPLETING FORM: ______
Please circle the most appropriate answer.
Do you live with the participant? No Yes
How much contact do you have with the participant? Less than 1 day per week 1 day/week 2 days/week 3-4 days/week
5 or more days per week
Relationship to participant
Self Spouse Sibling Child Other Family Friend Other ______

In each section, please circle the number that most closely applies to the participant. This is a general form, so no one description may be exactly right—please circle the answer that seems to apply most of the time.

Please circle only one number per section, and be sure to answer all questions.

### Memory

0 Normal memory.
1 Occasionally forgets things that they were told recently.
Does not cause many problems.
2 Mild consistent forgetfulness. Remembers recent events but often forgets parts.
3 Moderate memory loss. Worse for recent events. May not remember something you just told them. Causes problems with everyday activities.
4 Substantial memory loss. Quickly forgets recent or newly-learned things. Can only remember things that they have known for a long time.
5 Does not remember basic facts like the day of the week, when last meal was eaten or what the next meal will be.
6 Does not remember even the most basic things.

### Speech and Language

0 Normal ability to talk and to understand others.
1 Sometimes cannot find a word, but able to carry on conversations.
2 Often forgets words. May use the wrong word in its place. Some trouble expressing thoughts and giving answers.
3 Usually answers questions using sentences but rarely starts a conversation.
4 Answers questions, but responses are often hard to understand or don't make sense. Usually able to follow simple instructions.
5 Speech often does not make sense. Can not answer questions or follow instructions.
6 Does not respond most of the time.

### Recognition of Family Members

0 Normal-recognizes people and generally knows who they are.
1 Usually recognizes grandchildren, cousins or relatives who are not seen frequently but may not recall how they are related.
2 Usually does not recognize family members who are not seen frequently. Is often confused about how family members such as grandchildren, nieces, or nephews are related to them.
3 Sometimes does not recognize close family members or others who they see frequently. May not recognize their children, brothers, or sisters who are not seen on a regular basis.
4 Frequently does not recognize spouse or caregiver.
5 No recognition or awareness of the presence of others.

### Orientation to Time

0 Normal awareness of time of day and day of week.
1 Some confusion about what time it is or what day of the week, but not severe enough to interfere with everyday activities.
2 Frequently confused about time of day.
3 Almost always confused about the time of day.
4 Seems completely unaware of time.

### Orientation to Place

0 Normal awareness of where they are even in new places.
1 Sometimes disoriented in new places.
2 Frequently disoriented in new places.
3 Usually disoriented, even in familiar places. May forget that they are already at home.
4 Almost always confused about place.

### Ability to Make Decisions

0 Normal—as able to make decisions as before.
1 Only some difficulty making decisions that arise in day-to-day life.
2 Moderate difficulty. Gets confused when things get complicated or plans change.
3 Rarely makes any important decisions. Gets confused easily.
4 Not able to understand what is happening most of the time.

### Social and Community Activity

0 Normal-acts the same with people as before
1 Only mild problems that are not really important, but clearly acts differently from previous years.
2 Can still take part in community activities without help. May appear normal to people who don't know them.
3 Often has trouble dealing with people outside the home without help from caregiver. Usually can participate in quiet home activities with friends. The problem is clear to anyone who sees them.
4 No longer takes part in any real way in activities at home involving other people. Can only deal with the primary caregiver.
5 Little or no response even to primary caregiver.

### Home Activities and Responsibilities

0 Normal. No decline in ability to do things around the house.
1 Some problems with home activities. May have more trouble with money management (paying bills) and fixing things. Can still go to a store, cook or clean. Still watches TV or reads a newspaper with interest and understanding.
2 Makes mistakes with easy tasks like going to a store, cooking or cleaning. Losing interest in the newspaper, TV or radio. Often can't follow a long conversation on a single topic.
3 Not able to shop, cook or clean without a lot of help. Does not understand the newspaper or the TV. Cannot follow a conversation.
4 No longer does any home-based activities.

### Personal Care-Cleanliness

0 Normal. Takes care of self as well as they used to.
1 Sometimes forgets to wash, shave, comb hair, or may dress in wrong type of clothes. Not as neat as they used to be.
2 Requires help with dressing, washing and personal grooming.
3 Totally dependent on help for personal care.

### Eating

0 Normal, does not need help in eating food that is served to them.
1 May need help cutting food or have trouble with some foods, but basically able to eat by themselves.
2 Generally able to feed themselves but may require some help. May lose interest during the meal.
3 Needs to be fed. May have trouble swallowing.

### Control of Urination and Bowels

0 Normal—does not have problems controlling urination or bowels except for physical problems.
1 Rarely fails to control urination (generally less than one accident per month).
2 Occasional failure to control urination (about once a week or less).
3 Frequently fails to control urination (more than once a week).
4 Generally fails to control urination and frequently can not control bowels.
   Ability to Get from Place to Place
0 Normal, able to get around on their own. (May have physical problems that require a cane or walker).
1 Sometimes gets confused when driving or taking public transportation, especially in new places. Able to walk places alone.
2 Cannot drive or take public transportation alone, even in familiar places. Can walk alone outside for short distances. Might get lost if walking too far from home.
3 Cannot be left outside alone. Can get around the house without getting lost or confused.
4 Gets confused and needs help finding their way around the house.
5 Almost always in a bed or chair. May be able to walk a few steps with help, but lacks sense of direction.
6 Always in bed. Unable to sit or stand.

### Author:

Dr. Christopher M Clark, Alzheimer's Disease Core Center
Department of Neurology, University of Pennsylvania, Philadelphia, Pennsylvania, USA

### 6.2.3 Cognitive Evaluations

Cognitive testing included evaluations of the ADAS-Cog and Montreal-Cognitive Assessment (MoCA).

### ADAS-Cog

The ADAS-Cog is validated and widely used as a primary cognitive outcome measure in AD pharmacotherapy studies. This is a psychometric instrument that evaluates memory (immediate and delayed word recall, word recognition), attention (number cancellation), reasoning (following commands), language (naming, comprehension), orientation, ideational praxis (placing letter in envelope) and constructional praxis (copying geometric designs), and executive functioning (maze completion). Scoring is in the range of 0 to 90 with a higher score indicating greater impairment. This test was administered by experienced raters at each site at Baseline, Week 12, Week 24/Early Discontinuation Visits. The ADAS-Cog was the primary cognitive outcome measure for this study.

If the ADAS-Cog was administered remotely, it was completed to the extent possible given the available technology. If the ADAS-Cog was not completed remotely, then it was documented as a minor deviation.

### MoCA

Montreal Cognitive Assessment (MoCA) is commonly utilized questionnaire in clinical trials and research settings to measure levels of cognitive impairment. The MoCA measures five areas of cognitive function: orientation, visuospatial, attention and calculation, recall, and language. The MoCA will take approximately 10 minutes to complete. The test was administered by experienced raters at each site at Screening, Week 6, Week 18, Week 24/Early Discontinuation Visit. See below for the worksheet.

The 3 available versions of the MoCA test were administered by experienced raters at each site. Subjects were given any version of the MoCA at the Screening Visit as long as they had NOT received the same MoCA version clinically within the last 3 months. A different version of the MoCA was used at each subsequent visit until Week 24 or Early Termination visit (if applicable). The MoCA version used at a study visit was accurately documented in Source Document and within the EDC for each visit.

Re-screening: In cases where the subject screen failed due to the MoCA score being out of the required inclusionary range at the Screening Visit, the subject was re-screened; a minimum of one month must have passed since their original MoCA assessment. If the subject's MoCA score was <10, they had to have had a change in therapy or intervention that (in the opinion of the SI) may have had an impact on the subject's cognitive status. Subjects who moved forward with a re-screen were evaluated using a different MoCA version (7.1, 7.2 or 7.3) that was used at the original Screening Visit, and subjects were re-screened only once.

If the MoCA was administered remotely, it should be completed to the extent possible given the available technology. If the MoCA was not completed remotely, then it was documented as a minor deviation.

### 6.2.4 Neuropsychiatric Inventory Questionnaire (NPI-Q)

The Neuropsychiatric Inventory (NPI) measures dementia-related behavioral symptoms and was used to assess changes in psychological status. There are several versions of the NPI including the NPI-Questionnaire (NPI-Q), NPI-Clinician (NPI-C) and the NPI-Nursing Home (NPI-NH). All examine 12 sub-domains of behavioral functioning including: hallucinations, delusions, agitation, dysphoria, anxiety, euphoria, apathy, disinhibition, irritability, aberrant motor activity, eating abnormalities, and night-time behavioral alterations. The NPI-Q was completed by a trained rater through interview with the subject's study partner. It was well-validated and extensively used in clinical trials in AD. The NPI-Q was administered at the Screening or Baseline, Week 12 and Week 24/Early Discontinuation Visits.

### 6.2.5 Lumbar Puncture for CSF Biomarker Levels

Cerebrospinal fluid (CSF) levels biomarkers related to cellular processes and pathways implicated in Alzheimer's disease and mechanisms of action of AMX-0035 were evaluated. Biomarker analysis may include Aβ₁₋₄₂, total-τ, phospho-τ, NfL, YKL-40, Neurogranin, MCP-1, IL-6, and mitochondrial redox markers pyruvate, lactate and 8-OHdG. CSF samples were collected prior to AMX0035 treatment, anytime between the screening visit and up to 7 days prior to the baseline visit the baseline visit and following 24 weeks of treatment at the final study visit or at the early discontinuation visit.

Subjects had a fasting morning CSF sample taken anytime between the Screening and up to 7 days prior to the Baseline Visit (pre-dose) and the Week 24 and/or the Early Discontinuation Visit. If the Early Discontinuation Visit occurred within 7 days after the initiation of study drug (Baseline Visit), then the LP did not need to be completed. Lumbar punctures (LPs) were performed by qualified, experienced practitioners. Standard protocols were used employing sterile conditions, local lidocaine anesthesia, and preferred use of the Sprotte 24-gauge needles, which minimized incidence of post-LP headaches. Approximately 15-20 cc of CSF were collected for CSF biomarker assays, as well as routine chemistry (protein, glucose) and cell count.

### 6.2.6 Neuroimaging

MRI Scans: In accordance with secondary study objectives, all subjects underwent 3T structural and functional MRI was completed anytime between the Screening and up to 7 days prior to Baseline Visit and Week 24 and/or the Early Discontinuation Visits to assess the effect of treatment on brain volume, cerebral perfusion, and brain connectivity. Each scan session lasted approximately 35 minutes and included the following MR pulse sequences: high resolution T1-weighted multi-echo MPRAGE for volumetric analysis, task-free blood oxygen level dependent (BOLD) sequence to measure resting neural connectivity, diffusion tensor imaging (DTI) to quantify white matter structural connectivity, susceptibility-weighted imaging (SWI), and a T2-weighted fluid attenuated inversion recovery (FLAIR) sequence. Radiologist at each site provided a clinical read. For volumetric analysis, high resolution T1-weighted images underwent reconstruction and volumetric segmentation using the Freesurfer image analysis suite. All imaging and biomarker processing and assessment was done in a blinded fashion.

### Volumetric Analyses Include:

Total gray matter volume
Total, left, and right hippocampal volume
Parahippocampal volume
Ventricular and choroid plexus volume
Average cortical thickness

### White Matter Analyses Include:

Number of white matter lesions
Volume of white matter lesions

### CSF and Plasma Biomarkers

Pathognomic Alzheimer's Disease Markers—Amyloid Beta₁₋₄₂, Amyloid Beta₁₋₄₀, Total Tau, Phospho (p)-Tau 181

Alzheimer's disease is defined by the presence of abundant amyloid plaques and the presence of neurofibrillary tangles in neuronal cortices. These pathologic lesions are composed primarily of particular Aβ species and tau, respectively, and extensive studies have established that CSF measures of these proteins are sensitive and specific diagnostic markers of AD (Blennow K, Mattsson N, Schöll M, Hansson O, Zetterberg H. Amyloid biomarkers in Alzheimer's disease. Trends Pharmacol Sci 2015; 36:297-309. https://doi.org/https://doi.org/10.1016/j.tips.2015.03.002 Further, these markers may serve as prognostic biomarkers of progression of cognitive decline in MCI and AD.

Aβ₁₋₄₂/Aβ₁₋₁₀ ratio

The Aβ₁₋₄₂/Aβ₁₋₄₀ ratio is a key marker in Alzheimer's and decreases in the CSF in patients with Alzheimer's Disease. Studies have shown that the CSF Aβ₁₋₄₂/Aβ₁₋₄₀ is a more reliable indicator of AD than other typical AD biomarkers. It has also been suggested that the ratio can be used to differentiate between types of dementia. See, e.g., Hansson, O., Lehmann, S., Otto, M. et al. Advantages and disadvantages of the use of the CSF Amyloid β (Aβ) 42/40 ratio in the diagnosis of Alzheimer's Disease. Alz Res Therapy 11, 34 (2019). https://doi.org/10.1186/s13195-019-0485-0 and James D. Doecke, Virginia Pérez-Grijalba, Noelia Fandos, Christopher Fowler, Victor L. Villemagne, Colin L. Masters, Pedro Pesini, Manuel Sarasa, for the AIBL Research Group, “Total Aβ42/Aβ40 ratio in plasma predicts amyloid-PET status, independent of clinical AD diagnosis,” Neurology April 2020, 94 (15) e1580-e1591; DOI: 10.1212/WNL.0000000000009240, incorporated herein by reference

### Neuroinflammation

A panel of neuroinflammatory biomarkers was assessed, including but not limited to YKL-40, MCP-1, IL-6, GFAP, etc. This biomarker panels reflect both innate and/or adaptive immune activation and will broadly allow insight into the effects of AMX0035 at targeting all components of neuroimmunity.

### Neurodegeneration-Neurofilament Light Chain (NfL), Neurogranin (Ng)

Neurofilament-light change is a putative marker of subcortical large-caliber axonal degeneration and has been shown to be elevated in subjects with AD and MCI (Mattsson N, Insel P S, Palmqvist S, Portelius E, Zetterberg H, Weiner M, et al. Cerebrospinal fluid tau, neurogranin, and neurofilament light in Alzheimer's disease. EMBO Mol Med 2016; 8:1184-96. https://doi.org/https://doi.org/10.15252/emmm.201606540). Furthermore, levels of CSF NfL were found to be associated with more rapid AD disease progression. Levels of CSF NfL are thought to be biomarkers of non-specific axonal degeneration and have been demonstrated to be elevated in subjects with inflammatory disease (Mattsson N, Insel P S, Palmqvist S, Portelius E, Zetterberg H, Weiner M, et al. Cerebrospinal fluid tau, neurogranin, and neurofilament light in Alzheimer's disease. EMBO Mol Med 2016; 8:1184-96. https://doi.org/https://doi.org/10.15252/emmm.201606540), Creutzfeldt-Jakob disease (Constantinescu R, Krýsl D, Bergquist F, Andrén K, Malmeström C, Asztély F, et al. Cerebrospinal fluid markers of neuronal and glial cell damage to monitor disease activity and predict long-term outcome in patients with autoimmune encephalitis. Eur J Neurol 2016; 23:796-806. https://doi.org/https://doi.org/10.1111/ene.12942), progressive supranuclear palsy (Hall S, Öhrfelt A, Constantinescu R, Andreasson U, Surova Y, Bostrom F, et al. Accuracy of a Panel of 5 Cerebrospinal Fluid Biomarkers in the Differential Diagnosis of Patients With Dementia and/or Parkinsonian Disorders. Arch Neurol 2012; 69:1445-52. https://doi.org/10.1001/archneurol.2012.1654), ALS and vascular dementia

Neurogranin (Ng) is a calmodulin-binding post-synaptic protein thought to be expressed exclusively in the brain and particularly enriched in dendritic spines (Petersen A, Gerges N Z. Neurogranin regulates CaM dynamics at dendritic spines. Sci Rep 2015; 5:11135. https://doi.org/10.1038/srep11135). Ng is hypothesized to play a role in long-term potentiation and memory consolidation (Portelius E, Zetterberg H, Skillbäck T, Törnqvist U, Andreasson U, Trojanowski J Q, et al. Cerebrospinal fluid neurogranin: relation to cognition and neurodegeneration in Alzheimer's disease. Brain 2015, 138:3373-85. https://doi.org/10.1093/brain/awv267). CSF Ng levels are increased in AD and correlated with levels of “core” AD CSF biomarkers (Liu W, Lin H, He X, Chen L, Dai Y, Jia W, et al. Neurogranin as a cognitive biomarker in cerebrospinal fluid and blood exosomes for Alzheimer's disease and mild cognitive impairment. Transl Psychiatry 2020; 10:125. https://doi.org/10.1038/s41398-020-0801-2).

### Oxidative Stress-8-Hydroxy-2 Deoxyguanosine (8-OHdG)

8-OHdG is a biomarker indicating oxidative DNA damage that has been shown to be elevated in AD and potentially positively correlated with duration of illness (Isobe C, Abe T, Terayama Y. Levels of reduced and oxidized coenzyme Q-10 and 8-hydroxy-2′-deoxyguanosine in the CSF of patients with Alzheimer's disease demonstrate that mitochondrial oxidative damage and/or oxidative DNA damage contributes to the neurodegenerative process. J Neurol 2010; 257:399-404. https://doi.org/10.1007/s00415-009-5333-x). This molecule was used to assess DNA damage.

Metabolism—Leptin, Soluble Insulin Receptor (sIR), 24-Hydroxycholesterol (24-OHC)

Soluble insulin receptor is a component of metabolic critical to the detection and uptake of glucose. Levels of sIR are thought to be elevated as a sign of insulin resistance (Gerena Y, Menéndez-Delmestre R, Skolasky R L, Hechavarria R M, Pérez S, Hilera C, et al. Soluble insulin receptor as a source of insulin resistance and cognitive impairment in HIV-seropositive women. J Neurovirol 2015; 21:113-9. https://doi.org/10.1007/s13365-014-0310-2). Elevated levels of sIR have been associated with cognitive dysfunction (Gerena Y, Menéndez-Delmestre R, Delgado-Nieves A, Vélez J, Mendez-Alvarez J, Sierra-Pagan J E, et al. Release of Soluble Insulin Receptor From Neurons by Cerebrospinal Fluid From Patients With Neurocognitive Dysfunction and HIV Infection. Front Neurol 2019; 10:285. https://doi.org/10.3389/fneur.2019.00285).

24-OHC is a metabolite of brain cholesterol measurable in CSF and blood (Hughes T M, Rosano C, Evans R W, Kuller L H. Brain cholesterol metabolism, oxysterols, and dementia. J Alzheimers Dis 2013; 33:891-911. https://doi.org/10.3233/JAD-2012-121585). The majority of brain cholesterol is stored in myelin. Myelin disruption and breakdown is thought to lead to increased production of 24-OHC in the brain, and elevated levels of 24-OHC in fluids. Previous studies have found that levels of 24-OHC were higher in early compared to later stages of AD, suggesting that 24-OHC may be a useful marker in early stages of dementia (Papassotiropoulos A, Lütjohann D, Bagli M, Locatelli S, Jessen F, Rao M L, et al. Plasma 24S-hydroxycholesterol: a peripheral indicator of neuronal degeneration and potential state marker for Alzheimer's disease. Neuroreport 2000; 11:1959-62. https://doi.org/10.1097/00001756-200006260-00030).

### Neurovascular Injury—MMP-10

MMP-10 is a biomarker of neurovascular injury. MMP10 is highly produced in neurons of damaged compared to healthy brain tissue (Cuadrado E, Rosell A, Penalba A, Slevin M, Alvarez-Sabin J, Ortega-Aznar A, et al. Vascular MMP-9/TIMP-2 and neuronal MMP-10 up-regulation in human brain after stroke: a combined laser microdissection and protein array study. J Proteome Res 2009; 8:3191-7. https://doi.org/10.1021/pr801012x). Moreover, elevated levels of CSF MMP10 have been associated with tau and p-tau levels in individuals with Alzheimer's (Duits F H, Hernandez-Guillamon M, Montaner J, Goos J D C, Montañola A, Wattjes M P, et al Matrix Metalloproteinases in Alzheimer's Disease and Concurrent Cerebral Microbleeds. J Alzheimers Dis 2015; 48:711-20. https://doi.org/10.3233/JAD-143186).

### Additional Biomarkers

Additional biomarker panels, focused on the possible involvement of neurovascular injury and histone deacetylation, will likely be performed in CSF and plasma.

Broad proteomic panels was investigated in a hypothesis-generating manner. At present, we plan to work with protein biomarker panels that leverage proximity extension and next-generation sequencing to provide highly multiplexed immunoassays.

### 6.2.7 Pharmacokinetics

Plasma concentrations of both TUDCA and PB were assessed at the Week 12 and Week 24 (approximately at the same time as CSF sample, if taken on the same day). The time of the previous two drug administrations and the time of the sample were noted in the eCRF. A PK sample was not taken at an Early Discontinuation Visit if the subject discontinued study drug more than 48 hours before the visit.

### 6.2.8 Training and Validation

All evaluators were certified by the study PI to perform cognitive and psychiatric outcome assessments. It was strongly preferred that a single evaluator performs all measures with a given instrument throughout the study, if possible.

### 7. Safety and Adverse Events

The adverse event (AE) definitions and reporting procedures provided in this protocol comply with all applicable United States Food and Drug Administration (FDA) regulations and International Conference on Harmonization (ICH) guidelines. The Site Investigator will carefully monitor each subject throughout the study for possible adverse events. All AEs were documented on CRFs designed specifically for this purpose. It is also important to report all AEs, especially those that result in permanent discontinuation of the investigational product being studied, whether serious or non-serious.

### 7.1 Definitions of AEs, Suspected Adverse Drug Reactions & SAEs

### 7.1.1 Adverse Event and Suspected Adverse Drug Reactions

An adverse event (AE) is any unfavorable and unintended sign (including a clinically significant abnormal laboratory finding, for example), symptom, or disease temporally associated with a study, use of a drug product or device whether or not considered related to the drug product or device.

Adverse drug reactions (ADR) are all noxious and unintended responses to a medicinal product related to any dose. The phrase “responses to a medicinal product” means that a causal relationship between a medicinal product and an adverse event is at least a reasonable possibility, i.e., the relationship cannot be ruled out. Therefore, a subset of AEs can be classified as suspected ADRs, if there is a suspected causal relationship to the medicinal product.

Examples of adverse events include: new conditions, worsening of pre-existing conditions, clinically significant abnormal physical examination signs (i.e. skin rash, peripheral edema, etc.), or clinically significant abnormal test results (i.e. lab values or vital signs), with the exception of outcome measure results, which are not being recorded as adverse events in this trial (they are being collected, but analyzed separately). Stable chronic conditions (i.e., diabetes, arthritis) that are present prior to the start of the study and do not worsen during the trial are NOT considered adverse events. Chronic conditions that occur more frequently (for intermittent conditions) or with greater severity, would be considered as worsened and therefore would be recorded as adverse events.

Adverse events are generally detected in two ways:
Clinical—symptoms reported by the subject or signs detected on examination.
Ancillary Tests—abnormalities of vital signs, laboratory tests, and other diagnostic procedures (other than the outcome measures, the results of which are not being captured as AEs).

For the purposes of this study, symptoms of clinically noteworthy progression/worsening of cognitive, behavioral or functional abilities were recorded as AEs.

The following measures of disease progression will not be recorded as AEs even if they worsen (they are being recorded and analyzed separately): ADAS-Cog, DSRS, and FAQ.

If discernible at the time of completing the AE log, a specific disease or syndrome rather than individual associated signs and symptoms should be identified by the Site Investigator and recorded on the AE log. However, if an observed or reported sign, symptom, or clinically significant laboratory anomaly is not considered by the Site Investigator to be a component of a specific disease or syndrome, then it should be recorded as a separate AE on the AE log. Clinically significant laboratory abnormalities, such as those that require intervention, are those that are identified as such by the SI.

Subjects were monitored for AEs from the time they provide and sign consent until completion of their participation in the study (defined as death, consent withdrawal, loss to follow up, early study termination for other reasons or following completion of the entire study). Any treatment AE still present upon completion of treatment (including early discontinuation) should be monitored until resolution or until the AE is declared a chronic condition.

An unexpected adverse event is any AE in which the specificity or severity of which is not consistent with the current Investigator's Brochure. An unexpected, suspected adverse drug reaction is any unexpected adverse event that, in the opinion of the SI or Sponsor, has a reasonable possibility of being related to the investigational product.

### 7.1.2 Serious Adverse Events

A serious adverse event (SAE) is defined as an adverse event that meets any of the following criteria:
1. Results in death.
2. Is life threatening: that is, poses an immediate risk of death as the event occurred.
   a. This serious criterion applies if the study subject, in the view of the Site Investigator or Sponsor, is at immediate risk of death from the AE as it occurs. It does not apply if an AE hypothetically might have caused death if it were more severe.
3. Requires inpatient hospitalization or prolongation of existing hospitalization.
   a. Hospitalization for an elective procedure or a routinely scheduled treatment is not an SAE by this criterion because an elective or scheduled “procedure” or a “treatment” is not an untoward medical occurrence.
4. Results in persistent or significant disability or incapacity.
   a. This serious criterion applies if the “disability” caused by the reported AE results in a substantial disruption of the subject's ability to carry out normal life functions.
5. Necessitates medical or surgical intervention to preclude permanent impairment of a body function or permanent damage to a body structure.
6. Important medical events that may not result in death, are not life-threatening, or do not require hospitalization may also be considered SAEs when, based upon appropriate medical judgment, they may jeopardize the subject and may require medical or surgical intervention to prevent one of the outcomes listed in this definition. Examples of such medical events include blood dyscrasias or convulsions that do not result in inpatient hospitalization, or the development of drug dependency or drug abuse.

An inpatient hospital admission in the absence of a precipitating, treatment-emergent, clinical adverse event may meet criteria for “seriousness” but is not an adverse experience, and will therefore not be considered an SAE. An example of this would include a social admission (subject admitted for other reasons than medical, e.g., lives far from the hospital, has no place to sleep).

A serious, suspected adverse drug reaction is an SAE that, in the opinion of the SI or Sponsor, there is a reasonable possibility is related to the investigational product. The SI is responsible for classifying adverse events as serious or non-serious.

### 7.2 Assessment and Recording of Adverse Events

The Site Investigator will carefully monitor each subject throughout the study for possible AEs. All AEs were documented on source document templates and eCRFs designed specifically for this purpose. All AEs were reported in the EDC system and compiled into reports for periodic reviewing by the Medical Monitor. The Medical Monitor shall promptly review all information relevant to the safety of the investigational product, including all serious adverse events (SAEs). Special attention was paid to those that result in permanent discontinuation of the investigational product being studied, whether serious or non-serious.

### 7.2.1 Assessment of Adverse Events

At each visit (including telephone interviews), the subject was asked if they have had any problems or symptoms since their last visit in order to determine the occurrence of adverse events. If the subject reports an adverse event, the Site Investigator will probe further to determine:
1. Type of event
2. Date of onset and resolution (duration)
3. Severity (mild, moderate, severe)
4. Seriousness (does the event meet the above definition for an SAE)
5. Causality, relation to investigational product and disease
6. Action taken regarding investigational product
7. Outcome

### 7.2.2 Relatedness of Adverse Event to Investigational Product

The relationship of the AE to the investigational product should be specified by the Site Investigator, using the following definitions:
1. Not Related: Concomitant illness, accident, or event with no reasonable association with treatment.
2. Unlikely: The reaction has little or no temporal sequence from administration of the investigational product, and/or a more likely alternative etiology exists.
3. Possibly Related: The reaction follows a reasonably temporal sequence from administration of the investigational product and follows a known response pattern to the suspected investigational product; the reaction could have been produced by the investigational product or could have been produced by the subject's clinical state or by other modes of therapy administered to the subject. (Suspected ADR)
4. Probably Related: The reaction follows a reasonably temporal sequence from administration of investigational product, is confirmed by discontinuation of the investigational product or by re-challenge; and cannot be reasonably explained by the known characteristics of the subject's clinical state. (Suspected ADR)
5. Definitely Related: The reaction follows a reasonable temporal sequence from administration of investigational product; that follows a known or expected response pattern to the investigational product; and that is confirmed by improvement on stopping or reducing the dosage of the investigational product, and reappearance of the reaction on repeated exposure. (Suspected ADR)
   7.2.3 Adverse Events in Prior Human Experience with Each Individual Component

Both TUDCA and PB have been evaluated individually in multiple patient populations. The most commonly reported adverse events are below:

### TUDCA:

A small number of subjects receiving TUDCA have presented with mild diarrhea, abdominal and pain
Rare incidences of skin rash and vomiting have been observed

### PB

Menstrual irregularities, decreased appetite, sweat-like body odor, and bad taste Less common side effects include: nausea, vomiting, stomach upset, stomach pain, gastritis, headache, and skin rash. Rarely, cases of peptic ulcers, rectal bleeding, constipation, pancreatitis and renal tubular acidosis have been reported.
Hypoalbuminemia, metabolic acidosis, alkalosis, hyperchloremia, hyperuricemia, hypokalemia, bypophosphatemia, hyperphosphatemia and hypernatremia have been observed.

### 7.2.4 Recording of Adverse Events

All clinical adverse events are recorded in the Adverse Event (AE) Log in the subject's study binder. The site should fill out the AE Log and enter the AE information into the EDC system within 48 hours of the site learning of a new AE or receiving an update on an existing AE.

Serious Adverse Events (SAEs) must be reported to the MGH Coordination Center within 24 hours of the site learning of the SAE.

Entries on the AE Log (and into the EDC) included the following: name and severity of the event, the date of onset, the date of resolution, relationship to investigational product, action taken, and primary outcome of event.

### 7.3 Adverse Events and Serious Adverse Events-Reportable Events

The following are considered reportable events and must be reported to the MGH Coordination Center within 24 hours of the site being notified of the event.
All events that meet the above criteria for Serious Adverse Events (SAEs)
Dosage Changes (Dose Management)
   Investigational Product Suspension, Reduction or Re-challenge
   Investigational Product Discontinuation

### 8. Data Safety Monitoring and Statistical Analysis Plan

### 8.1 Medical Monitoring

A Medical Monitor independent of the trial conduct was identified by the Study Sponsor. The Medical Monitor's responsibilities included a regular evaluation of the frequency, severity and type of AEs and SAEs reported by all sites in the study. The Medical Monitor was a physician with expertise in Alzheimer's disease and common chronic medical (e.g., renal and cardiac) conditions in this elderly patient population and with prior experience with the conduct of clinical trials (trial design, safety monitoring, and recruitment). All AEs were collected and reported in the electronic data capture (EDC) system and compiled into bi-monthly blinded reports for periodic reviewing by the Medical Monitor. Any possibly, probably or definitely study drug related, serious adverse events (i.e. suspected unexpected serious adverse drug reactions, or SUSARs) and any death are considered events of interest and was reported in real-time (within 1 business day of Coordination Center (CC) awareness) to the Medical Monitor and to members of the PSC. The Medical Monitor will approve the format and content of the periodic safety report. The Medical Monitor shall promptly review all information relevant to the safety of the investigational product, including all serious adverse events (SAEs) and AEs unusual in the context of Alzheimer disease. Special attention was paid to those that result in permanent discontinuation of the investigational product being studied, whether serious or non-serious. The Medical monitor may ask to receive the AE reports more frequently. The Medical Monitor will communicate their recommendations to the PSC.

### 8.2 Protocol Steering Committee

The Protocol Steering Committee (PSC) is composed of the Principal Investigator of the study (serving as SC Chair), the study biostatistician, independent Investigator members with expertise in Alzheimer's disease and study-related medical (e.g., renal and cardiac) conditions and priorities (trial recruitment and drug supply) and the Sponsor Acting Medical Director. The PSC is responsible for the design of the study protocol and analysis plan and oversees the clinical trial from protocol development to study analysis and publication.

### 8.3 Statistical Methods

Complete details of efficacy and safety analyses is provided in the section “Analysis for Efficacy.”

### 8.3.1 Sample-Size Determination

A sample size of approximately 100 randomization subjects was chosen based on feasibility and is not based solely in statistical considerations. Subjects were randomized in a ratio of 3:2 (active versus placebo). In order to maximize study power for evaluation of efficacy end-points, a global test statistic was used. The global test statistic was a combination of 3 change-from-baseline to end-of-study endpoints (univariate components), including the following:
Cognition
Activities of Daily Living (ADL)
Total Hippocampal Brain Volume

The global test statistic was calculated for each subject as a mean percentile score across the above 3 component endpoints for each subject in the study. This mean percentile score will then be analyzed as the primary efficacy outcome variable. For purposes of power calculations, global test statistics can be characterized based on the assumed effect sizes of each of the 3 component endpoints and the correlations between each of the 3 pairs of component endpoints.

Use of a global test statistic can handle the case of correlated univariate endpoints and provide substantially greater power than the use of univariate test statistics, even when the component univariate endpoints have unequal magnitudes of treatment effects in favor of the active treatment. We assume a correlation of 0.4 between the cognitive and ADL component endpoints and a correlation of 0.2 between the total hippocampal volume component endpoint and each of the other two components endpoints (i.e., the cognitive component endpoint and the ADL component endpoint), based upon historical analyses. By using 50% statistical power and the assumed effect sizes as inputs, we can obtain the “expected p-value” for 2 selected combinations of component endpoints of the composite. We do this in 2 stages, first for the combination of ADAS-Cog and ADL component endpoints, which have expected p-values of 0.09409 and 0.20184, respectively, resulting in a 2-component expected p-value of 0.077583. Then we combine the 2-component expected p-value with the expected p-value for Total Hippocampal Brain Volume (0.20184), to get a global test statistic expected p-value of 0.049756.

Each of the 3 component scales may have a different sensitivity with respect to detecting change over time. The sensitivity is quantified using the Mean-to-Standard Deviation Ratio (MSDR), which is calculated by dividing the mean of the change-from-baseline score by the standard deviation of the change-from-baseline score. For change-from-baseline to 6 months, we assume the MSDR values for ADAS-Cog, ADL, Total Hippocampal Volume are 0.8, 0.6, and 0.6, respectively. We assume a 60% value for Percent of Placebo Effect (i.e., the decline for the active treatment group is only 40% of the decline of the Placebo group). This corresponds to effect size (assumed treatment difference divided by the common standard deviation) values of 0.48 for the Cognitive Endpoint, 0.36 for the ADL endpoint, and 0.36 for the Total Hippocampal Brain Volume endpoint. As described previously, using these expected p-values and the assumed correlations between each pair of these 3 endpoints results in an expected p-value of 0.049756 for the global test statistic.

Using 100 randomized subjects (assuming either no subject dropout so that there are 100 completers, or that imputation appropriately accounts for subject dropout), 50% power, a randomization ratio of 1:1, gives an effect size (corresponding to the use of the global test statistic) of 0.566; this indicates that that under these assumptions use of the global test statistic is equivalent to using a single component test statistic with an effect size of 0.566 (rather than the assumed effect sizes of 0.48 for ADAS-Cog, 0.36 for ADL, and 0.36 for Total Hippocampal Brain Volume). Another way to interpret this is that using of the global test statistic, corresponding to an assumed effect size of 0.566 for a single component endpoint, and using α=0.05 results in a power of approximately 50% (50.1%). So, when the individual components of the global test statistic are in the same direction (and in favor of the active treatment group versus placebo), then use of a global test statistic generally provides more power than use of a single component.

Although the power calculations (see table below, Table 2) show 80% power for an assumed Percent of Placebo Effect value of 85%, a Percent of Placebo Effect value as small as 50% or 60% would still be clinically relevant and would be an encouraging indication for moving forward into a new study. As described previously, a Percent of Placebo Effect value of 60% corresponds to global test statistic p-value of approximately 0.05 (0.049756, 2-sided), an effect size of 0.566, and a power of approximately 50%. A global test statistic p-value of 0.10 (2-sided) would be an observed trend that would still be suggestive of a signal indicating that moving forward into a new study would be appropriate. This corresponds to Percent of Placebo Effect value of just under 50%: a Percent of Placebo Effect value of 50% corresponds to global test statistic p-value of approximately 0.095 (0.09477, 2-sided), an effect size of 0.479, and a power of approximately 38% at a 2-sided alpha=0.05 level.

TABLE 2

| | | | | | | | Cog. & ADL | Global Test | | |
|---|---|---|---|---|---|---|---|---|---|---|
| | | | Total Hipp. | Cog. | ADL | Total Hipp. | Combined | Statistic | | Global Test |
| % of | Cog. | ADL | Brain | Expected | Expected | Expected | Expected | Expected | Global Test | Statistic |
| Placebo | Effect | Effect | Volume | p-value | p-value | p-value | p-value | p-value | Statistic | Power |
| Effect | Size | Size | Effect Size | (2-sided) | (2-sided) | (2-sided) | (2-sided) | (2-sided) | Effect Size | (%) |
| 50% | 0.40 | 0.30 | 0.30 | 0.15959 | 0.27395 | 0.27395 | 0.135094 | 0.09477 | 0.479 | 38.3 |
| 60% | 0.48 | 0.36 | 0.36 | 0.09409 | 0.20184 | 0.20184 | 0.077583 | 0.049756 | 0.566 | 50.1 |
| 70% | 0.56 | 0.42 | 0.42 | 0.05206 | 0.14709 | 0.14709 | 0.042619 | 0.024794 | 0.651 | 61.7 |
| 80% | 0.64 | 0.48 | 0.48 | 0.02727 | 0.09409 | 0.09409 | 0.020349 | 0.009933 | 0.754 | 74.4 |
| 85% | 0.68 | 0.51 | 0.51 | 0.01937 | 0.07588 | 0.07588 | 0.013962 | 0.006283 | 0.803 | 79.5 |
| 90% | 0.72 | 0.54 | 0.54 | 0.01358 | 0.06069 | 0.06069 | 0.009432 | 0.003896 | 0.852 | 84.0 |
| 100% | 0.80 | 0.60 | 0.60 | 0.00646 | 0.03793 | 0.03793 | 0.004131 | 0.001417 | 0.951 | 90.9 |

Some additional examples of the global test statistic under various assumed Percent of Placebo Effect values and corresponding effect sizes (and using the pairwise correlations of 0.4, 0.2, and 0.2 described previously) are given below, along with the examples described previously. (Note that a Percent of Placebo effect value of 0% means that active treatment declines as much as Placebo, a value of 50% means that active treatment declines half as much as Placebo, and a value of 100% means that the active treatment doesn't decline at all over the 6-month treatment period.)

### 8.3.2 Study Unblinding

After database lock, the responsible statistician will request the treatment codes, the study was unblinded, and the statistical analysis was conducted.

### 8.3.3 Safety Endpoints

### 8.3.3.1 Safety Endpoints

The primary safety end-point was the incidence of treatment emergent Grade II-IV adverse events.

Other safety endpoints are:
Incidence and severity of treatment emergent adverse events (AEs)
Clinical laboratory tests
Vital signs
Physical examinations
ECGs
Use of concomitant medications for treatment of AEs
C-SSRS

### 8.3.3.2 Primary Efficacy Endpoints

The primary efficacy endpoint was a global test statistic (GST) for change from Baseline to 24 weeks as described in the section “Sample-Size Determination.” Briefly, the GST combines correlated univariate components (MADCOMS, FAQ, and total hippocampal volume), providing greater power to detect treatment effect than any of the univariate components alone. A correlation of 0.4 between the cognitive and FAQ components and 0.2 between the hippocampal volume and each of the other two components was assumed based on historical analyses.

### 8.3.3.3 Secondary Efficacy Endpoints

The change in the following assessments from Screening and/or Baseline to each post-baseline visit as described below based on the Schedule of Activities (see below, Table 3) between the AMX0035 treatment group and the placebo treatment group in the ITT population:
Cognition Endpoint component for the global test statistic (Baseline, Week 12, and Week 24)
ADL Endpoint component for the global test statistic (Baseline, Week 12, and Week 24)
Total Hippocampal Brain Volume (Screening/Baseline and Week 24)
Additional volumetric MRI brain volume parameters, (Screening/Baseline and Week 24)
ADAS-Cog (Baseline, Week 12, and Week 24)
FAQ (Baseline, Week 12, and Week 24)
DSRS (Baseline, Week 12, and Week 24)
NPI-Q (Screening/Baseline, Week 12, and Week 24)
MoCA (Screening, Week 6, Week 18, and Week 24)

TABLE 3
SCHEDULE OF ACTIVITIES

| | | | Week 1 | | | | | Final Follow- |
|---|---|---|---|---|---|---|---|---|
| | | | Phone | | | | Week 24¹⁷/Early | Up Call¹⁴ |
| | | Baseline¹ | Call | Week 6¹⁵ | Week 12¹⁶ | Week 18¹⁵ | Discontinuation¹⁸ | Last Dose of |
| | Screening | Day 0 + | Day 7 ± | Day 42 ± | Day 84 ± | Day 126 ± | Day 168 ± | IP + |
| | −28 Days | 5 Days | 1 Days | 14 Days | 28 Days | 14 Days | 28 Days | 14 ± 5 Days |
| Written Informed consent | X | | | | | | | |
| Inclusion/Exclusion Review | X | X | | | | | | |
| Randomization² | | X | | | | | | |
| Medical History/Demographics | X | | | | | | | |
| AD Diagnosis History | X | | | | | | | |
| Montreal - Cognitive Assessment | X | | | X | | X | X | |
| (MoCA) | | | | | | | | |
| DSRS | | X | | | X | | X | |
| Geriatric Depression Scale | X | | | | | | | |
| Vital Signs³ | X | X | | | X | | X | |
| FAQ | | X | | | X | | X | |
| Physical Exam including Height | X | | | | | | X | |
| and Weight⁴ | | | | | | | | |
| Neurology Exam⁵ | X | | | | | | X | |
| Safety labs⁶ | X | | | | X | | X | |
| 12-Lead ECG (Electrocardiogram) | X | | | | X | | X | |
| Neuropsychiatric Inventory | X⁷ | | | | X | | X | |
| Questionnaire (NPI-Q) | | | | | | | | |
| ADAS-Cog | | X | | | X | | X | |
| MRI Assessment⁸ | X | | | | | | X | |
| Adverse Events | X | X | X | X | X | X | X | X |
| Concomitant Medications | X | X | | X | X | X | X | X |
| Dispense Study Drug⁹ | | X | | X | X | X | | |
| Drug Accountability/Compliance | | | X¹⁰ | X | X | X | X | |
| Suicide Rating Scale (C-SSRS)¹¹ | X | X | | X | X | X | X | |
| Blood draw for biomarker analysis | | X | | | X | | X | |
| Lumbar puncture/CSF draw for | X | | | | | | X | |
| biomarkers¹² | | | | | | | | |
| Blood draw for pharmacokinetics¹³ | | | | | X | | X | |
| Blood draw for genetic analysis | | X | | | | | | |

¹The Baseline Visit can be completed any time after the screening so long as all eligibility criteria are met and occur no more than 28 + 5 days after the Screening Visit.
²Randomization should occur at the Baseline Visit. Randomization will entail entering a subject's kit number into the electronic data capture system.
³Vital signs include systolic and diastolic pressure in mmHg, respiratory rate/minute, heart rate/minute and temperature.
⁴Height is only recorded once at the Screening Visit.
⁵The standard Neurological Exam will be used for all subjects.
⁶Safety labs include Hematology (CBC with differential), Complete Chemistry Panel, Liver Function Tests, B12 and TSH (at Screening Visit only) and Urinalysis.
⁷The NPI-Q Test can be performed either at the screening or baseline visit
⁸The MRI assessment can be completed anytime between the Screening and up to 7 days prior to the Baseline Visit and will have a clinical read done locally.
⁹First dose of study drug will be administered in clinic after ALL Baseline Visit procedures are completed.
¹⁰Notify subjects of increase from one sachet per day to two sachets per day
¹¹C-SSRS Screening Version to be completed at Screening Visit only. C-SSRS Since Last Visit version to be completed at all other visits.
¹²The first LP can be completed anytime between the Screening and up to 7 days prior to the Baseline Visit.
¹³Take a single PK plasma sample on Visits 12 and 24 (same time as lumbar puncture). A PK sample will not be taken on subjects completing the Early Discontinuation Visit if the subject discontinued study drug more than 48 hours before the visit.
¹⁴The Final Follow-Up Call is to occur 14 ± 5 days after the participant's last dose of study drug. For participants who discontinue treatment early, the Final Follow-Up Call is not required if the Early Discontinuation visit occurs 14 ± 5 days after the last dose of study drug.
¹⁵To the extent possible, the Week 6 and Week 18 visits may be done remotely. If the visit is done remotely, Drug Accountability/Compliance will occur at the next in-person clinic visit.
¹⁶It is preferred that the Week 12 visit be conducted at the site with the participant physically present. If it is not possible to complete an in-person Week 12 visit at the site, the safety assessments below must be completed by Week 16/Day 112 for the subject to remain on study drug. Sites may make alternative arrangements to complete these assessments per institutional and IRB policy.
ECG
Safety Labs: Hematology (Complete Blood Count with Differential), Complete Chemistry Panel, Liver Function Tests, and Urinalysis
Vital Signs
C-SSRS
¹⁷The window for the Week 24 visit is Day 168 ± 28 days. However, if COVID-19 related restrictions (e.g., site closure, travel restrictions) make it impossible to conduct an in-person clinic visit during the specified window, then any assessment that can be performed remotely should be completed as an unscheduled visit during the Week 24 window. The actual Week 24 visit, with all of the assessments indicated on the SOA, may be postponed for up to 12 weeks and treatment extended. The maximum duration a subject may be on IP is 40 weeks. Safety checks (i.e., ECG, safety labs, vital signs, and C-SSRS) must be completed, at minimum, every 16 weeks/112 days. If safety assessments are performed so that the Week 24 visit may be postponed, the assessments should be documented as an unscheduled visit.
¹⁸If possible, an Early Discontinuation visit should be done within 14 days of the last dose of IP (i.e., last dose of IP + 14 days). The MRI and lumbar puncture may be done as soon as is practical and, if possible, within 60 days of the last dose of IP (i.e., last dose of IP + 60 days).

Derivation for the Cognition Endpoint component and the ADL Endpoint component for the global test statistic was designed to optimize sensitivity in the MCI and mild-to-moderate AD populations by using scales specific to each of these two populations (with some overlapping tests and some tests distinct to each population. The process of combining scores for each scale (i.e., Cognition Endpoint component and ADL Endpoint component) across the two populations is described in the section “Global Statistical Test”.

A Mixed-effects Model for Repeated Measures was used to calculate differences at 12 and 24 weeks, using baseline assessment as a covariate.

Secondary efficacy endpoints were assessed using MMRM. For each of the 3 component efficacy endpoints (based on Cognition, ADL, and Total Hippocampal Brain Volume) which are used to calculate the global test statistic, a sensitivity analysis using Pattern Mixture Models (PMM) as described in the section “Missing Data” was done to impute missing data at Week 24.

### 8.3.3.4 Volumetric MRI

Observed Case analysis for differences in change of vMRI over 24 weeks. Ventricular volume and hippocampal volume were measured. MRI imaging of the brain was performed in order to measure brain atrophy over time. Imaging was performed using cross-sectional approach for baseline and week 24 samples as well as using the longitudinal approach for baseline and week 24 pairs. Results from vMRI studies suggest that the patterns of atrophy in AD can reliably be detected and tracked across time. Hippocampal volume derived from MRI correlates with histological hippocampal volume and degree of neuronal loss and AD pathology. Longitudinal MRI measures of regional and whole brain volumetric change provide a valuable complement to cognitive measures in that they are not influenced by temporary symptomatic improvements, and they may provide an early index of the study drug's ability to reach the central nervous system and effect AD-related atrophy.

### 8.3.3.5 CSF

Observed Case analysis for differences in change of CSF biomarkers over 24 weeks.

### 8.3.4 Analysis Populations

The safety population included all randomized subjects who received at least 1 dose of study medication. Subjects in the safety population was analyzed based on the treatment they actually received, and not necessarily the one to which they were randomized

An intent-to-treat (ITT) approach was used to define the primary efficacy analysis population. For a given endpoint, all randomized subjects who received any study medication, had a baseline assessment, and had at least 1 post-baseline efficacy assessment for the primary efficacy endpoint was included in the ITT population. In the ITT population, subjects who switched treatment groups over the course of the study were analyzed based on their randomized treatment, and not necessarily the treatment they actually received.

### 8.3.5 Analysis for Safety

The Safety population was used for analyses of each of the safety endpoints. All concomitant medications were tabulated according to drug class and preferred term using the WHO dictionary. The safety data was summarized by treatment group. Treatment AEs were coded and graded using MedDRA grading criteria. The treatment groups were compared with respect to occurrence of each adverse event and incidence of Grade II/IV adverse events. Withdrawal, abnormal laboratory tests, vital signs and use of concomitant medications used for treatment of AEs were assessed to characterize the safety profile of the combination of PB and TUDCA. Compliance data was determined for each visit and by treatment group. The time to subject refusal was compared between treatment groups to better determine tolerability. This was accomplished using a method of survival analysis that allows informative censoring due to death. Descriptive statistics denoting the changes from baseline to the final assessment visit with respect to key laboratory parameters and vital signs will also be provided.

### 8.3.5.1 Adverse Events

Adverse events (AE) occurring after the start of study drug dosing at Baseline were summarized descriptively for the safety population. All AEs were coded according to system organ class (SOC) and preferred term (PT) using a Medical Dictionary for Regulatory Activities (MedDRA) dictionary. Summary tables showing the number of subjects and percent within each category were generated for each of the following types of adverse events and its relationship to study treatment (related to study treatment):
All events
Serious events
Deaths
Events leading to withdrawal
Severe events

Treatment AEs were coded and graded using MedDRA grading criteria. The treatment groups were compared with respect to occurrence of each adverse event and incidence of Grade II/IV adverse events. Total number of adverse events were compared between groups using Fisher's exact test. Any treatment AE still present upon completion of treatment (including early discontinuation) should be monitored until resolution or until the AE is declared a chronic condition. AEs were monitored until they become chronic or have completely resolved.

### 8.3.5.2 Laboratory Parameters

Laboratory parameters were summarized by visit. Descriptive statistics denoting the changes from baseline to the final assessment visit with respect to key laboratory parameters and vital signs will also be provided. Frequencies of high and low values with respect to the normal range were displayed, as will shift tables comparing each treatment visit and Baseline visit by time point and treatment group. Abnormal laboratory tests were compared between groups using Fisher's exact test.

### 8.3.5.3 Other Safety Parameters

Vital signs were summarized across groups by visit using descriptive statistics, and at each outcome visit and at end of study Physical examination findings and number of subjects were summarized as the count and percentage of subjects by eCRF pre-defined categories at last visit. Change from baseline at last visit were summarized in a shift table comparing baseline and last visit results. Concomitant medications were summarized by treatment group, drug class and preferred term. The change in the C-SSRS score from Baseline to each post-baseline visit were summarized between the active treatment group and the placebo treatment group.

### 8.3.6 Analysis for Efficacy

Analysis of primary and secondary efficacy endpoints were performed on the ITT population (LZCF and non-LZCF).

The primary efficacy analysis was based on the use of a global test statistic as described in the section “Sample-Size Determination”. The null hypothesis was that there is no difference between the treatment groups, and the corresponding alternative hypothesis is that treatment with AMX0035 will result in a statistically significant difference (in favor of the active treatment group) in the global test statistic score relative to the placebo group at Week 24 in the ITT population. As described in in the section “Sample-Size Determination”, the global test statistic p-value was calculated using the 3 individual component p-values (corresponding to change from baseline to Week 24 for the Cognition Endpoint, ADL Endpoint, and Total Hippocampal Brain Volume) and the 3 pairwise correlations values between the 3 endpoints.

The primary efficacy analysis using the global test statistic was based on using the Pattern Mixture Model (PMM) approach described in the section “Missing Data” in order to impute any missing data at Week 24 for the 3 component endpoints (i.e., Cognition Endpoint, ADL Endpoint, and Total Hippocampal Brain Volume). A secondary analysis using the global test statistic was based on using observed cases.

The GST was calculated for each subject as a mean zscore across the 3 component endpoints (as mentioned above under section “Sample-Size Determination”) for each subject in the study. This mean score will then be analyzed as the primary efficacy outcome variable.

GST individual items with right censored data was imputed using LZCF. Intermittently missing data was imputed using straight line imputation. Straight line imputation or z-scores were calculated for each data collection timepoint in the study (baseline, week 6, week 12, week 18, week 24). Let Z be the imputed z-score, while x is the last observation at timepoint t₁ and μ and σ are the mean and standard deviation of the next timepoint (t₂) for which the data is missing.

$${Z}_{t 2} = ( {x}_{t 1} - {μ}_{t 2} ) / {σ}_{t 2}$$

Z-scores imputed relative to the group mean and standard deviation at each timepoint, will better preserve the slope of each arm and thus is more robust to differences in dropout rate across treatment. Individuals missing all post-baseline data will use the population baseline zscore to calculate LZCF by treatment group. GST were calculated for each individual after LZCF is calculated for individual level items. LZCF imputations were constrained by the range of the possible score for each measure.

A composite covariate was calculated for the primary efficacy variable (GST) to adjust for baseline covariates as well as those that interact with time and were included in the model. To calculate the composite, the change score (GST) is regressed on time. The residuals are regressed on the covariates listed below. Individual composite coefficients for each covariate are multiplied by individual covariate values and summed. A separate covariate composite is calculated for baseline covariates and baseline covariates interacting with time.

CFB was analyzed by comparing the change between treatment group using a mixed model with repeated measures (MMRM). The MMRM will compare the estimated change from baseline between treatments for each primary endpoint. This analysis will assess whether there is a difference in estimated CFB between active and placebo groups.

The MMRM with primary outcome CFB value as the response variable included the following covariates and fixed effects:
Baseline composite covariate only:
Age (covariate);
baseline ADAS-Cog
baseline MOCA
baseline FAQ
baseline DSRS
baseline Hippocampal volume
AD/dementia status (fixed effect);
Concomittant use of AD medications
Level of Education (fixed effect split into categories of ≤12 years, >12 years);
Sex (fixed effect);
APOEε4 status (fixed effect, positive or negative);
Time composite covariate only:
time
Age*time;
baseline ADAS-Cog*time
baseline MOCA*time
baseline FAQ*time
baseline DSRS*time
baseline Hippocampal volume*time
AD/dementia status*time;
Concomittant use of AD medications*time
Level of Education*time (fixed effect split into categories of ≤12 years, >12 years);
Sex*time;
APOEε4 status*time (positive or negative);
Time (continuous time);
Time by treatment interaction (Time*Treatment);
Baseline Test Score of Efficacy Parameter (covariate);
Site (random effect);

The covariance structure for the repeated measures in this model was unstructured (UN). If UN does not converge for the model, the MMRM model was simplified to allow convergence as described in the following paragraph. Variance components were used as the covariance structure for the random site effect in the model.

Any efficacy outcomes that do not converge using the specified primary model were rerun using a first-order heterogeneous autoregressive (ARH[1]) covariance structure, and then a compound symmetry (CS) followed by variance components (VC) structures if ARH (1) doesn't converge. The covariance structure for the site random effect was VC.

Least-squares means were estimated at each visit for the primary outcome. The LS mean at the endpoint was interpreted as the expected CFB in the primary outcome at the 24 week estimate drawn from the model within each group. Least squares means and standard errors were estimated from the mixed model at all timepoints (week 12 and 24) and were shown for all analyses. In addition, treatment differences, p-values, 95% confidence intervals for the difference, effect size, 95% confidence interval for the effect size, and an effect size based upon Cohen's D were displayed for each comparison. Effect size was calculated by taking the difference of LSMEANS and dividing by the standard deviation (i.e. the standard error of the estimated difference multiplied by the squared degrees of freedom). The equations below show how effect size and Cohen's D effect size were calculated. In the following formulas p stands for placebo group and/stands for treated), SE for standard error and df for degrees of freedom:

$$Effect Size = \frac{{LSMEAN}_{t} - {LSMEAN}_{p}}{{LSMEAN}_{p}}$$

Cohen's d was calculated using the following equation:

$$Cohen ’ s d = \frac{{μ}_{t} - {μ}_{p}}{( pooled SD )} ,$$

where the pooled standard deviation (pooled SD) is defined as follows:

$$Pooled SD = \sqrt{\frac{( ( {n}_{t} - 1 ) {( {ST}_{t} \sqrt{{n}_{t}} )}^{2} + ( ( {n}_{p} - 1 ) {( S {E}_{p} \sqrt{{n}_{p}} )}^{2}}{( {n}_{t} + {n}_{p} - 1 )}} .$$

The number of subjects with an observed efficacy outcome, mean, standard deviation, median, 25^{th} percentile (Q1), 75^{th} percentile (Q3), minimum and maximum will all be reported and accompany the estimates from the MMRM outlined in this section (Tables 14.2.1-14.2.16).

### Covariate and Categorical Interaction Analyses

The following variables were assessed for interactions with the primary efficacy variable and time using the MMRM as described above:
baseline score (for outcome being analyzed)
baseline MoCA score
ApoEε4 (carrier/non-carrier; discrete)
ApoEε4 (number of E4 alleles; discrete)

Estimates for continuous variables were drawn at the first and third quartiles

An MMRM without imputation was performed as a sensitivity analysis. The same MMRM parameters described above were used. Additionally, an MMRM was run for the primary analysis using MRI quantitative data generated with the longitudinal approach.

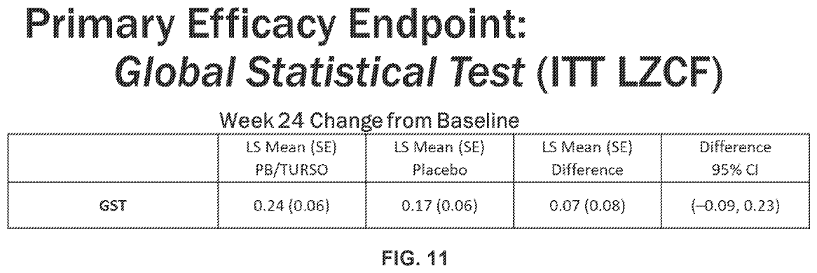

FIG. 11 shows the calculated GST for the Intent-to-Treat LZCF Population.

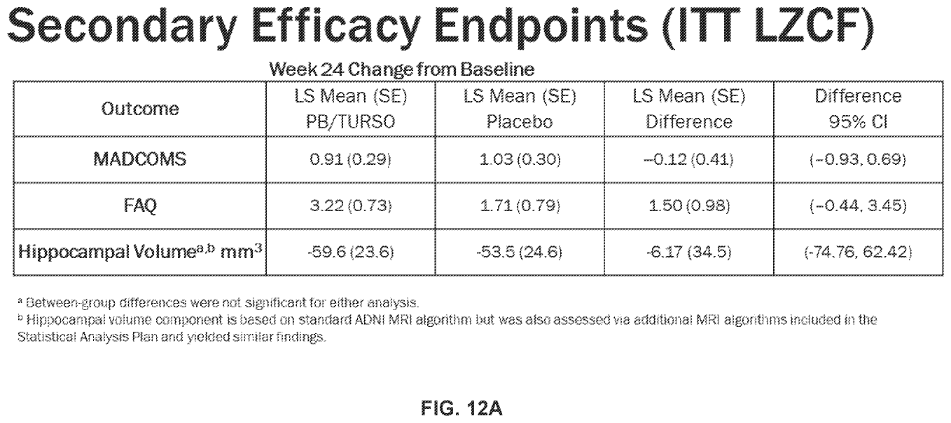

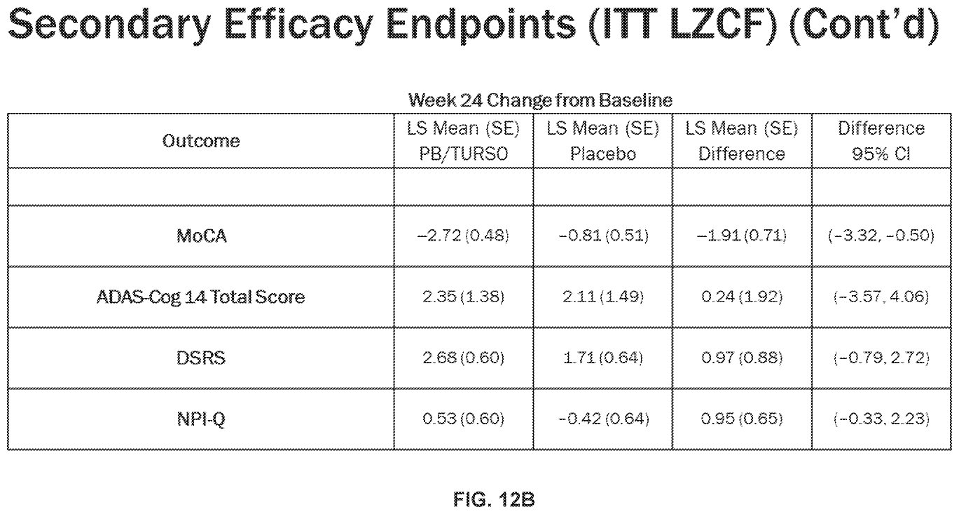

Analyses for secondary efficacy endpoints were conducted using mixed models for repeated measures (MMRM). The mixed model analysis will compare the estimated change from baseline (CFB), or change from screening (as applicable), between active treatment and placebo in all efficacy outcome scores at each scheduled post-baseline visit. Separate repeated measures longitudinal models were used for each efficacy endpoint. This analysis will assess whether there is a difference in estimated CFB values between treatment groups. SAS PROC MIXED were used to fit the MMRM models, with CFB of each of the efficacy outcomes (e.g., ADAS-cog Total Score) as the response variable and certain covariates and fixed effects as specified in the “Unblinding” section. See, e.g. FIGS. 12A and 12B.

### PK Analyses

PK was analyzed using the ITT population. Descriptive statistics were provided for the AMX0035 and placebo treatment groups. Additionally, correlations of exposure values to clinical outcomes may be assessed, if possible, by correlation to concentration data as well as summarization of outcomes data in the upper and lower PK tertiles.

The PK concentration data collected prior to each dose may be used in a concentration response relationship analysis to assess correlation with each outcome which will mirror the primary analysis but replace the treatment variable with PK concentration. Analysis may be performed separately for each PK collection time as well as using the higher of both PK timepoints.

Descriptive statistics for continuous variables included number of subjects (n), mean, standard deviation (SD), median, minimum, maximum, first and third quartiles, unless otherwise noted. Frequencies and percentages were calculated for categorical variables. Percentages were calculated within each treatment group on the number of non-missing observations.

Subgroup analysis (e.g., based on subgroups defined by gender, age, baseline MoCA score) were performed in the ITT population.

### Exploratory Analyses

Exploratory endpoints and biomarkers will also be assessed using the MMRM as described above.

Biomarker data excluded values with a large coefficient of variation. Additionally, biomarker analysis was divided into two distinct subgroups, clinically associated and target engagement, based on expected mechanism of AMX0035, with hierarchical analysis within each subgroup.

Markers of target engagement that were assessed in CSF include:
8-OHdG
Total Tau
Neurogranin
MMP-10
FABP3
NfL

Clinically associated markers that were assessed in CSF include:
Amyloid beta 1-42
Amyloid beta 1-40
P-Tau 181
24-OHC
YKL-40
GFAP
MCP-1.
IL-6, IL-7, IL-8, IL-15, IL-16, MDC, MIP-1B
Leptin
SIR

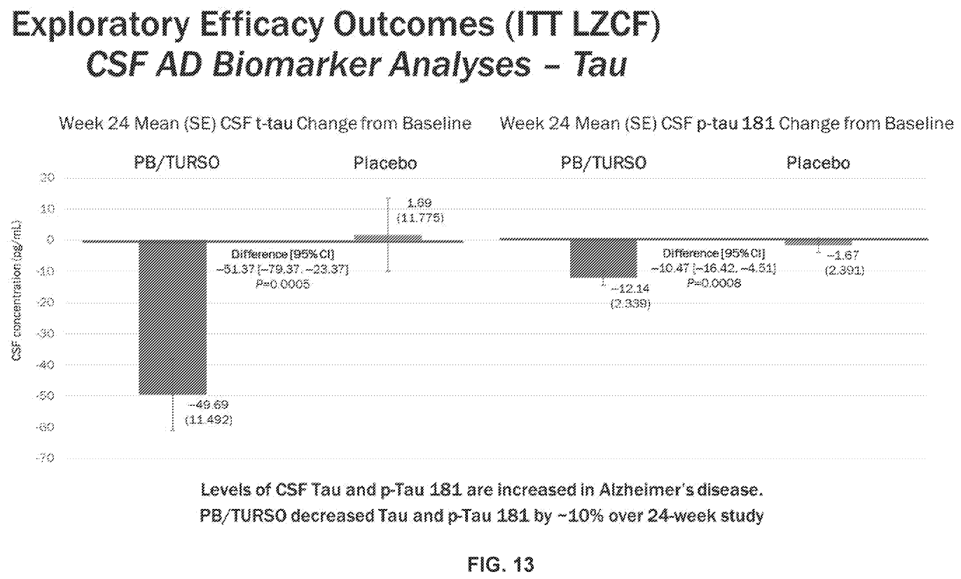

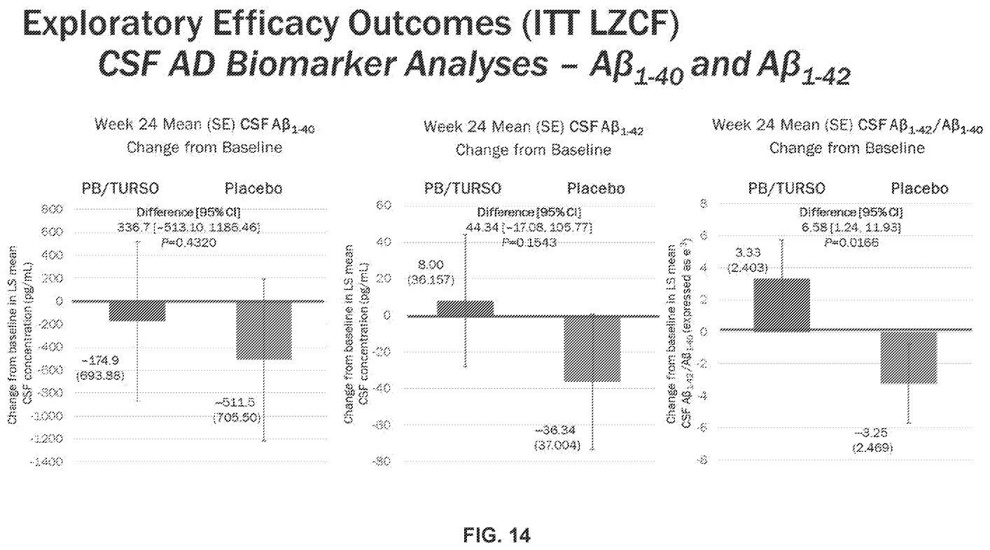

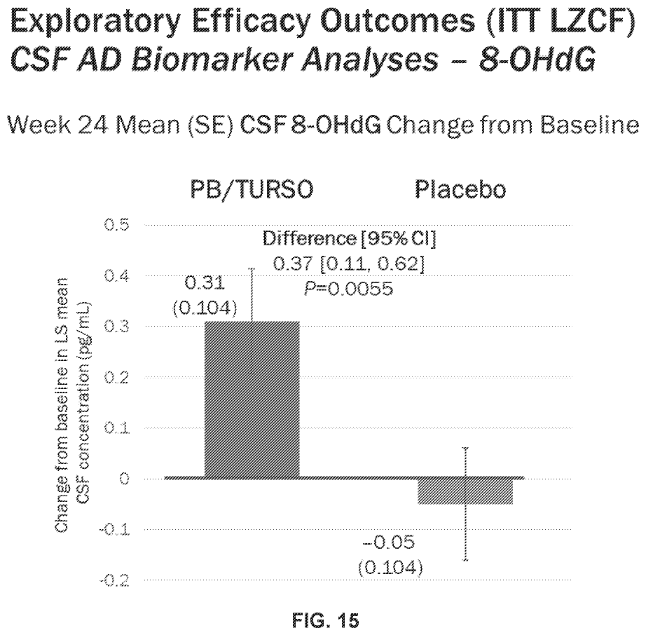

Biomarkers of target engagement were assessed in conjunction with pharmacokinetics in CSF and plasma. See, e.g. FIGS. 13-15 and Table 4. As shown, total tau and phospho-tau, which are both elevated in AD were significantly reduced in patients that were treated with AMX0035 as compared to the placebo group (see, e.g., FIG. 13 and Table 4). The treatment decreased CSF tau and p-Tau 181 by about 10% over the 24-week study. Patients that were treated with AMX0035 showed a significant increase in the ratio of Aβ₁₋₄₂ to Aβ₁₋₄₀ (third graph in FIG. 14). 8-OHdG, increased significantly in patients that were treated with AMX0035 (FIG. 15 and Table 4). Overall, the following was found: changes in core AD biomarkers were found to be significant, with reductions in CSF t-tau and p-tau and an increase in Aβ₁₋₄₂/Aβ₁₋₄₀, over the 24 weeks in the PB/TURSO group versus placebo group. Fatty acid-binding protein 3 (FABP3) was significantly reduced in the PB/TURSO group as compared to the placebo. There was also a significant reduction in Neurogranin in the PB/TURSO group versus the placebo group. Further, there was a decrease in inflammation markers, including for example, YKL-40 and IL-15, in the PB/TURSO group versus the placebo group.

TABLE 4

| | | Change from Baseline at Week 24 | | | |
|---|---|---|---|---|---|
| | | | | AMX0035 and Placebo LSMEAN | |
| | | AMX0035 | Placebo | Difference (95% CI) | p-value |
| Neurodegeneration* | Total Tau | −64 93 | 8.82 | −73.74 (−106.84, −40.65) | <0.0001 |
| (pg/mL) | Phosphorylated Tau | −14.63 | −0.27 | −14.36 (−21.51, −7.21) | 0.0002 |
| | (pTau 181) | | | | |
| | FABP3 | −344.62 | 102.90 | −447.52 (−684.59, −210.45) | 0.0004 |
| | Neurofilament Light | 169.48 | 63.61 | 105.87 (−119.74, 331.47) | 0.35 |
| | Chain (NfL) | | | | |
| Synaptic Function | Neurogranin (pg/mL) | −81.19 | −8.34 | −72.85 (−110.81, −34.89) | 0.0003 |
| Inflammation | YKL-40 | −14635.39 | 1507.88 | −16143.27 (−26995.89, −5290.65) | 0.004 |
| (pg/mL) | IL-15 | −0.02 | 0.25 | −0.28 (−0.49, −0.06) | 0.01 |
| | IL-6 | 644.38 | 565.93 | 78.45 (−1042.50, 1199.40) | 0.89 |
| | IL-8 | 1.54 | 1.17 | 0.37 (−4.37, 6.11) | 0.88 |
| | GFAP | 821.68 | 488.15 | 333.53 (−2080.17, 2747.22) | 0.78 |
| | MCP-1 | −1.97 | −0.79 | −1.18 (−21.15, 18.79) | 0.91 |
| Core | AB₄₂/AB₄₀ ratio | 0.0039 | −0.0051 | 0.0090 (0.0029, 0.0151) | 0.005 |
| AD pathology | AB₄₂ (pg/mL) | −8.09 | −41.46 | 33.37 (−38.37, 105.11) | 0.36 |
| | AB₄₀ (pg/mL) | −752.70 | −754.81 | 2.11 (−1007.67, 1011.88) | 1.0 |
| Metabolism/ | 8-OHdG | 0.31 | −0.13 | 0.44 (0.13, 0.74) | 0.006 |
| Oxidative Stress | 24-OHC | −0.20 | −0.07 | −0.13 (−0.67, 0.41) | 0.63 |
| (pg/mL) | Leptin | 0.45 | 4.53 | −4.09 (−25.71, 17.54) | 0.71 |
| | sIR | −0.04 | −0.19 | 0.15 (−0.25, 0.55) | 0.47 |
| Neurovascular | MMP-10 (pg/mL) | −3.13 | −0.92 | −2.21 (−8.48, 4.05) | 0.48 |

*Tau and Phosphorylated Tau may be considered Core AD Pathology

In addition to CSF and plasma biomarker analysis, exploratory assessments of MRI data were conducted. These analyses included analyses of grey matter white matter, resting state functional MRI (rsfMRI). In particular, the below analyses were conducted:
Whole brain volume
Ventricular volume
Mean cortical thickness
Total white matter volume
White matter lesion volume
Posterior cingulate default mode network (DMN) connectivity
   Biomarkers were further analyzed in an exploratory fashion to assess colinear changes and pathway enrichment.

In short, a total of 95 participants were randomized (PB/TURSO, n=51; placebo, n=44). The average participant age was 70.7 years; approximately 80% of participants were receiving concomitant acetylcholinesterase inhibitors and 42%, concomitant memantine. Mean (SD) cognitive assessment scores indicated a significantly greater baseline level of cognitive impairment among those randomized to PB/TURSO versus placebo (ADAS-Cog14 total score, MOCA, and MADCOMS, all P<0.01). The PB/TURSO group had a numerically higher percentage of apolipoprotein E &4 carriers compared with the placebo group (77.1% vs 61.4%, respectively; P=0.12). Baseline values for all other measures were similar between groups. Treatment-emergent adverse events (TEAEs) were reported in 34 (67%) participants in the PB/TURSO group and 26 (59%) participants in the placebo group, with gastrointestinal events (primarily diarrhea) accounting for the greatest proportion of TEAEs in the PB/TURSO group (39% vs 14% in the placebo group). Ten of 51 (20%) participants in the PB/TURSO group and 2 of 44 (5%) participants in the placebo group discontinued study. No significant between-group differences were observed for the primary or secondary end points. Changes in core AD biomarkers were found to be significant, with reductions in CSF t-tau (P=0.0005) and p-tau (P=0.0008) over the 24 weeks in the PB/TURSO versus placebo group and an increase in Aβ₁₋₄₂/Aβ1-40 (P=0.017).

### 8.4 Missing Data

Subjects who dropped out had all available post-baseline data included in the analysis. In addition, some subjects may have had missing data, because of visits completed remotely during the COVID-19 pandemic. The mixed model for repeated measures is based on an assumption of Missing at Random (MAR) and is designed to handle right-censored data for subjects who drop out of the study. An additional analysis was performed as a sensitivity analysis in this study and was a z-score based pattern mixture model (PMM) approach. The PMM will use an MAR assumption, and this sensitivity analysis will use a subject's last observed value and the z-score of that observation as a carried forward value, assuming a pattern of progression similar to subjects within the same treatment group who completed each visit. At each subsequent visit, a value was imputed such that it has the same z-score relative to that subject's treatment group mean and standard deviation for completers at that visit. The first analysis is intended to estimate the treatment effect expected if all subjects continued on treatment. After imputation for the sensitivity analyses, the estimated change from baseline between active and placebo group was assessed by fitting an analysis of covariance (ANCOVA) model.

### 8.5 Stopping Rules

The Study PI will review safety data throughout the trial and may stop the trial for safety. Any death will lead to prompt review by the Medical Monitor and Global Study PI Two or more of the same SAE deemed probably or definitely related to study drug by Site Investigators, will lead to prompt review by the Medical Monitor and Study PI.

### REFERENCES

1. Zhou, Wenbo, et al. “Phenylbutyrate up-regulates the DJ-1 protein and protects neurons in cell culture and in animal models of Parkinson disease.” Journal of Biological Chemistry 286.17 (2011): 14941-14951.
2. Wright, Jerry M., et al. “Gene expression profile analysis of 4-phenylbutyrate treatment of IB3-1 bronchial epithelial cell line demonstrates a major influence on heat-shock proteins.” Physiological Genomics 16.2 (2004): 204-211.
3. Cuadrado-Tejedor, Mar, et al. “Phenylbutyrate is a multifaceted drug that exerts neuroprotective effects and reverses the Alzheimer s disease-like phenotype of a commonly used mouse model.” Current Pharmaceutical Design 19.28 (2013): 5076-5084.
4. Ricobaraza, Ana, et al. “Phenylbutyrate rescues dendritic spine loss associated with memory deficits in a mouse model of Alzheimer disease.” Hippocampus 22.5 (2012): 1040-1050.
5. Wiley, Jesse C., Christina Pettan-Brewer, and Warren C. Ladiges. “Phenylbutyric acid reduces amyloid plaques and rescues cognitive behavior in AD transgenic mice.” Aging Cell 10.3 (2011): 418-428.
6. Ricobaraza, Ana, et al. “Phenylbutyrate ameliorates cognitive deficit and reduces tau pathology in an Alzheimer's disease mouse model.” Neuropsychopharmacology 34.7 (2009): 1721.
7. Ryu, Hoon, et al. “Sodium phenylbutyrate prolongs survival and regulates expression of anti-apoptotic genes in transgenic amyotrophic lateral sclerosis mice.” Journal of Neurochemistry 93.5 (2005): 1087-1098.
8. Gardian, Gabriella, et al. “Neuroprotective effects of phenylbutyrate in the N171-82Q transgenic mouse model of Huntington's disease.” Journal of Biological Chemistry 280.1 (2005): 556-563.
9. Corbett, Grant T., Avik Roy, and Kalipada Pahan. “Sodium phenylbutyrate enhances Astrocytic neurotrophin synthesis via protein kinase C (PKC)-mediated activation of CAMP-response element-binding protein (CREB).” Journal of Biological Chemistry 288.12 (2013): 8299-8312.
10. Rodrigues, Cecília M P, et al. “Tauroursodeoxycholic acid prevents Bax-induced membrane perturbation and cytochrome C release in isolated mitochondria.” Biochemistry 42.10 (2003): 3070-3080.
11. Lo, Adrian C., et al. “Tauroursodeoxycholic acid (TUDCA) supplementation prevents cognitive impairment and amyloid deposition in APP/PSI mice.” Neurobiology of disease 50 (2013): 21-29.
12. Dionisio, Pedro A., et al. “Amyloid-β pathology is attenuated by tauroursodeoxycholic acid treatment in APP/PSI mice after disease onset.” Neurobiology of aging 36.1 (2015): 228-240.
13. Nunes, Ana F., et al. “TUDCA, a bile acid, attenuates amyloid precursor protein processing and amyloid-β deposition in APP/PSI mice.” Molecular neurobiology 45.3 (2012): 440-454.
14. Rodrigues, Cecilia M P, et al. “Tauroursodeoxycholic acid reduces apoptosis and protects against neurological injury after acute hemorrhagic stroke in rats.” Proceedings of the National Academy of Sciences 100.10 (2003): 6087-6092.
15 Castro-Caldas, M., et al. “Tauroursodeoxycholic acid prevents MPTP-induced dopaminergic cell death in a mouse model of Parkinson's disease.” Molecular neurobiology 46.2 (2012): 475-486.
16. Zhang, Yong-Jie, et al. “Aggregation-prone c9FTD/ALS poly (GA) RAN-translated proteins cause neurotoxicity by inducing ER stress.” Acta Neuropathologica 128.4 (2014): 505-524.
17. Cudkowicz, Merit E., et al. “Phase 2 study of sodium phenylbutyrate in ALS.” Amyotrophic Lateral Sclerosis 10.2 (2009): 99-106.
18. Borovecki, F., et al. “Genome-wide expression profiling of human blood reveals biomarkers for Huntington's disease.” Proceedings of the National Academy of Sciences of the United States of America 102.31 (2005): 11023-11028.
19. Hogarth, Penelope, Luca Lovrecic, and Dimitri Krainc. “Sodium phenylbutyrate in Huntington's disease: A dose-finding study.” Movement Disorders 22.13 (2007): 1962-1964.
20. Elia, A., et al. “Tauroursodeoxycholic acid in the treatment of patients with amyotrophic lateral sclerosis.” European Journal of Neurology 23.1 (2016): 45-52.
21. Min, Ju-Hong, et al. “Oral solubilized ursodeoxycholic acid therapy in amyotrophic lateral sclerosis: a randomized cross-over trial.” Journal of Korean Medical Science 27.2 (2012): 200-206.
22. Buphenyl Package Insert URL: http://www.horizonpharma.com/wp-content/uploads/2016/04/BUPHENYL_PI April_2016.pdf
23. Gilbert, Jill, et al. “A phase I dose escalation and bioavailability study of oral sodium phenylbutyrate in patients with refractory solid tumor malignancies.” Clinical Cancer Research 7.8 (2001): 2292-2300.
24. Gore, Steven D., et al. “Impact of prolonged infusions of the putative differentiating agent sodium phenylbutyrate on myelodysplastic syndromes and acute myeloid leukemia.” Clinical Cancer Research 8.4 (2002): 963-970.
25. Wey, Hsiao-Ying, et al. “Insights into neuroepigenetics through human histone deacetylase PET imaging.” Science Translational Medicine 8.351 (2016): 351ra106-351ra106.
26. Zetterberg, Henrik, et al. “Association of cerebrospinal fluid neurofilament light concentration with Alzheimer disease progression.” JAMA Neurology 73.1 (2016): 60-67.
27. Constantinescu, Radu, et al. “Cerebrospinal fluid markers of neuronal and glial cell damage in patients with autoimmune neurologic syndromes with and without underlying malignancies.” Journal of Neuroimmunology 306 (2017): 25-30.
28. van Eijk, Jeroen J J, et al. “CSF neurofilament proteins levels are elevated in sporadic Creutzfeldt-Jakob disease.” Journal of Alzheimer's Disease 21.2 (2010): 569-576.
29. Steinacker, Petra, et al. “Neurofilaments in blood and CSF for diagnosis and prediction of onset in Creutzfeldt-Jakob disease.” Scientific Reports 6 (2016).
30. Jabbari, Edwin, Henrik Zetterberg, and Huw R. Morris. “Tracking and predicting disease progression in progressive supranuclear palsy: CSF and blood biomarkers.” J Neurol Neurosurg Psychiatry (2017): jnnp-2017.
31. Craig-Schapiro, Rebecaa, et al. “YKL-40: a novel prognostic fluid biomarker for preclinical Alzheimer's disease.” Biological psychiatry 68.10 (2010): 903-912.
32. Comabella, Manuel, et al. “Cerebrospinal fluid chitinase 3-like 1 levels are associated with conversion to multiple sclerosis.” Brain 133.4 (2010): 1082-1093.
33. Bonneh-Barkay, Dafna, et al. “YKL-40 expression in traumatic brain injury: an initial analysis.” Journal of Neurotrauma 27.7 (2010): 1215-1223.
34. Janelidze, Shorena, et al. “Cerebrospinal fluid neurogranin and YKL-40 as biomarkers of Alzheimer's disease.” Annals of Clinical and Translational Neurology 3.1 (2016): 12-20.
35. Melah, Kelsey E., et al. “Cerebrospinal fluid markers of Alzheimer's disease pathology and microglia activation are associated with altered white matter microstructure in asymptomatic adults at risk for Alzheimer's disease.” Journal of Alzheimer's Disease 50.3 (2016): 873-886.
36. Hayashi, Yasunori. “Long-term potentiation: two pathways meet at neurogranin.” The EMBO Journal 28.19 (2009): 2859-2860.
37. Zhong, Ling, et al. “Increased prefrontal cortex neurogranin enhances plasticity and extinction learning.” Journal of Neuroscience 35.19 (2015): 7503-7508.
38. Portelius, Erik, et al. “Cerebrospinal fluid neurogranin: relation to cognition and neurodegeneration in Alzheimer's disease.” Brain 138.11 (2015): 3373-3385.
39. Posner K, (et. al), Columbia Classification Algorithm of Suicide Assessment (C-CASA): Classification of Suicidal Events in the FDA's Pediatric Suicidal Risk Analysis of Antidepressants, Am J Psychiatry, 2007, 164:1035-1043.
40. September 2010 US FDA Draft Guidance for Industry Suicidality: Prospective Assessment of Occurrence in Clinical Trials
41. TUDCA in new onset diabetes. Clinicaltrials.gov identifier NCT02218619.
42. TUDCA and PB: effect on ER stress and metabolism. Clinicaltrial gov identifier. NCT00771901.
43. Castilla-Rilo, J., Lopez-Arrieta, J., Bermejo-Pareja, F., Ruiz, M., Sanchez-Sanchez, F., & Trincado, R. (2007). Instrumental activities of daily living in the screening of dementia in population studies: a systematic review and meta-analysis. Int J Geriatr Psychiatry, 22 (9), 829-836. doi: 10.1002/gps. 1747
44. Clark, C. M., & Ewbank, D. C. (1996). Performance of the dementia severity rating scale: a caregiver questionnaire for rating severity in Alzheimer disease. Alzheimer Dis Assoc Disord, 10 (1), 31-39.
45. Mitchell, J. C., Dick, M. B., Wood, A. E., Tapp, A. M., & Ziegler, R. (2015). The utility of the Dementia Severity Rating Scale in differentiating mild cognitive impairment and Alzheimer disease from controls. Alzheimer Dis Assoc Disord, 29 (3), 222-228. doi: 10.1097/WAD.0000000000000057
46. Moelter, S. T., Glenn, M. A., Xie, S. X., Chittams, J., Clark, C. M., Watson, M., & Arnold, S. E. (2015). The Dementia Severity Rating Scale predicts clinical dementia rating sum of boxes scores. Alzheimer Dis Assoc Disord, 29 (2), 158-160. doi: 10.1097/WAD.0000000000000031
47. Pfeffer, R. I., Kurosaki, T. T., Harrah, C. H., Jr., Chance, J. M., & Filos, S. (1982). Measurement of functional activities in older adults in the community. J Gerontol, 37 (3), 323-329
48. Rikkert, M. G., Tona, K. D., Janssen, L., Burns, A., Lobo, A., Robert, P., . . . . Waldemar, G. (2011). Validity, reliability, and feasibility of clinical staging scales in dementia: a systematic review. Am J Alzheimers Dis Other Demen, 26 (5), 357-365. doi: 10.1177/1533317511418954
49. Teng, E., Becker, B. W., Woo, E., Knopman, D. S., Cummings, J. L., & Lu, P. H. (2010). Utility of the functional activities questionnaire for distinguishing mild cognitive impairment from very mild Alzheimer disease. Alzheimer Dis Assoc Disord, 24 (4), 348-353. doi: 10.1097/WAD.0b013e3181e2fc84
50. Xie, S. X., Ewbank, D. C., Chittams, J., Karlawish, J. H., Arnold, S. E., & Clark, C. M. (2009). Rate of decline in Alzheimer disease measured by a Dementia Severity Rating Scale. Alzheimer Dis Assoc Disord, 23 (3), 268-274. doi: 10.1097/WAD.0b013e318194a324

### OTHER EMBODIMENTS

It is to be understood that while the invention has been described in conjunction with the detailed description thereof, the foregoing description is intended to illustrate and not limit the scope of the invention, which is defined by the scope of the appended claims. The following are numbered embodiments intended to further illustrate, but not limit, the scope of the invention.

1. A method of treating at least one symptom of AD in a human subject, the method comprising administering to the human subject about 10 mg/kg to about 50 mg/kg of body weight of a bile acid or a pharmaceutically acceptable salt thereof, and about 10 mg/kg to about 400 mg/kg of body weight of a phenylbutyrate compound, wherein the human subject:
(a) carries one or more copies of the APOEε4 allele;
(b) has a cerebral spinal fluid (CSF) level of total tau of about 300 μg/mL or higher; or
(c) has a CSF or level of phospho-tau of about 70 μg/mL or higher; to thereby treat at least one symptom of AD in the human subject.

2 The method of embodiment 1, wherein the method comprises, prior to administration, a step of determining whether the human subject has at least one of the characteristics of (a)-(c).

3 The method of embodiment 1, wherein the human subject has a cerebral spinal fluid (CSF) level of total tau of about 300 μg/mL or higher.

4. The method of embodiment 1, wherein the human subject has a CSF level of phospho-tau of about 70 μg/mL or higher.

5. A method of slowing AD disease progression in a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, to thereby slow AD disease progression in the human subject.

6. A method of increasing survival time of a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, to thereby increase survival time of the human subject.

7. A method of decreasing the level of total CSF tau, decreasing the level of CSF phospho-tau, and increasing Aβ₁₋₄₂/Aβ₁₋₄₀ in a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, thereby decreasing the level of total CSF tau, decreasing the level of CSF phospho-tau, and increasing Aβ₁₋₄₂/Aβ₁₋₄₀ in the human subject.

8. A method of decreasing the level of total CSF tau of a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, thereby decreasing the level of total CSF tau of the human subject.

9. A method of decreasing the level of CSF phospho-tau of a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, thereby decreasing the level of CSF phospho-tau of the human subject.

10. The method of embodiment 9, wherein the phospho-tau species is phospho-tau 181.

11. A method of increasing the level of CSF 8-OHDG of a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, thereby increasing the level of CSF 8-OHDG of the human subject.

12. A method of decreasing the level of total CSF tau, decreasing the level of CSF phospho-tau, increasing Aβ₁₋₄₂/Aβ₁₋₄₀, and increasing the level of CSF 8-OHDG in a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, thereby decreasing the level of total CSF tau, decreasing the level of CSF phospho-tau, increasing Aβ₁₋₄₂/Aβ₁₋₄₀, and increasing the level of 8-OHDG in the human subject.

13. A method of treating and/or preventing a tauopathy in a human subject, the method comprising:
administering to the human subject about 10 mg/kg to about 50 mg/kg of body weight of a bile acid or a pharmaceutically acceptable salt thereof, and about 10 mg/kg to about 400 mg/kg of body weight of a phenylbutyrate compound, thereby treating or preventing the tauopathy in the human subject.

14. The method of embodiment 13, wherein the subject has a baseline CSF total tau level of about 300 μg/mL or higher.

15. The method of embodiment 13, wherein the tauopathy is progressive supranuclear palsy (PSP), frontotemporal lobar degeneration (FTLD-TAU), corticobasal degeneration, Pick's disease, argyrophilic grain disease, post-encephalitic parkinsonism, chronic traumatic encephalopathy, primary age-related tauopathy, stroke, traumatic brain injury, or Alzheimer's disease.

16. The method of embodiment 13, wherein the tauopathy is progressive supranuclear palsy

17. A method of treating and/or preventing an amyloidosis related condition in a human subject, the method comprising:
administering to the human subject about 10 mg/kg to about 50 mg/kg of body weight of a bile acid or a pharmaceutically acceptable salt thereof, and about 10 mg/kg to about 400 mg/kg of body weight of a phenylbutyrate compound, thereby treating or preventing the amyloidosis related condition in the human subject.

18. A method comprising:
administering to a human subject at risk for developing AD about 10 mg/kg to about 50 mg/kg of body weight of a bile acid or a pharmaceutically acceptable salt thereof and about 10 mg/kg to about 400 mg/kg of body weight of a phenylbutyrate compound, to thereby prevent or delay the onset of AD.

19. The method of embodiment 18, wherein the subject is determined to be at risk for developing AD by evaluating a level of a biomarker in a biological sample obtained from the subject.

20. The method of embodiment 19, wherein the biomarker is total tau or phospho-tau.

21. The method of embodiment 19, wherein the biological sample is CSF.

22 The method of embodiment 18, wherein the subject carries one or more copies of the APOEε4 allele.

23. The method of embodiment 18, wherein the subject carries one or more mutations in at least one gene selected from the group consisting of: APP, PSEN1, and PSEN2.

24. A method of decreasing the CSF levels of FABP3, neurogranin, YKL-40, or IL-15 in a human subject having one or more symptoms of AD, the method comprising:
administering to the subject about 10 mg/kg to about 50 mg/kg of body weight of a bile acid or a pharmaceutically acceptable salt thereof, and about 10 mg/kg to about 400 mg/kg of body weight of a phenylbutyrate compound, to thereby decrease the CSF levels of FABP3, neurogranin, YKL-40, or IL-15 in a human subject.

25. The method of any one of the proceeding embodiments, wherein the bile acid is taurursodiol (TURSO), ursodeoxycholic acid (UDCA), chenodeoxycholic acid, cholic acid, hyodeoxycholic acid, lithocholic acid, or glycoursodeoxycholic acid.

26. The method of any one of the proceeding embodiments, wherein the phenylbutyrate compound is 4-phenylbutyric acid (4-PBA), Glycerly Tri-(4-phenylbutyrate), phenylacetic acid, 2-(4-Methoxyphenoxy) acetic acid (2-POAA-OMe), 2-(4-Nitrophenoxy) acetic acid (2-POAA-NO2), 2-(2-Naphtbyloxy) acetic acid (2-NOAA), or pharmaceutically acceptable salts thereof.

27. The method of any one of the proceeding embodiments, wherein the method comprises administering to the human subject about 10 mg/kg to about 30 mg/kg of body weight of the bile acid.

28. The method of any one of the proceeding embodiments, wherein the method comprises administering to the human subject about 10 mg/kg to about 100 mg/kg of body weight of the phenylbutyrate compound.

29. The method of embodiment 28, wherein the method comprises administering to the human subject about 30 mg/kg to about 100 mg/kg of body weight of the phenylbutyrate compound.

30. The method of any one of the proceeding embodiments, wherein the bile acid and the phenylbutyrate compound are administered separately.

31. The method of any one of the proceeding embodiments, wherein the bile acid and the phenylbutyrate compound are administered concurrently.

32. The method of any one of the proceeding embodiments, wherein the bile acid and the phenylbutyrate compound are administered daily

33. The method of embodiment 32, wherein the bile acid and the phenylbutyrate compound are administered once a day, twice a day, or three times a day

34. The method of any one of the preceding embodiments, wherein the bile acid and the phenylbutyrate compound are administered once a day for 60 days or less.

35. The method of any one of the preceding embodiments, wherein the bile acid and the phenylbutyrate compound are administered once a day for 30 days or less.

36. The method of any one of the preceding embodiments, wherein the bile acid and the phenylbutyrate compound are administered twice a day for 60 days or less.

37. The method of any one of the preceding embodiments, wherein the bile acid and the phenylbutyrate compound are administered twice a day for 30 days or less.

38. The method of any one of the preceding embodiments, wherein the bile acid and the phenylbutyrate compound are administered twice a day for 60 days or more.

39. The method of any one of the preceding embodiments, wherein the bile acid and the phenylbutyrate compound are administered twice a day for 120 days or more.

40. The method of any one of the preceding embodiments, wherein the bile acid and the phenylbutyrate compound are administered once a day for at least 14 days followed by twice a day for at least 30 days.

41. The method of any one of the preceding embodiments, wherein the bile acid and the phenylbutyrate compound are administered once a day for about 21 days followed by twice a day for at least 30 days.

42. The method of any one of the proceeding embodiments, wherein the bile acid and the phenylbutyrate compound are administered orally.

43. The method of any one of the proceeding embodiments, wherein the bile acid and the phenylbutyrate compound are administered through a feeding tube

44. The method of any one of embodiments 1-42, wherein the bile acid and the phenylbutyrate compound are administered by bolus injection

45. The method of any one of the preceding embodiments, wherein each of the bile acid and the phenylbutyrate compound is formulated as a solution.

46. The method of any one of embodiments 1-44, wherein the bile acid and the phenylbutyrate compound are formulated in a single solution.

47. The method of any one of embodiments 1-44, wherein each of the bile acid and the phenylbutyrate compound is formulated as a powder.

48. The method of any one of embodiments 1-44, wherein the bile acid and the phenylbutyrate compound are formulated as a single powder formulation.

49. The method of any one of the preceding embodiments, wherein the bile acid is TURSO.

50. The method of embodiment 49, wherein the TURSO is administered at an amount of about 0.5 to about 5 grams per day.

51. The method of embodiment 49, wherein the TURSO is administered at an amount of about 1.5 to about 2.5 grams per day.

52. The method of embodiment 49, wherein the TURSO is administered at an amount of about 1 gram twice a day.

53. The method of any one of the proceeding embodiments, wherein the phenylbutyrate compound is a pharmaceutically acceptable salt of 4-PBA.

54. The method of embodiment 52, wherein the pharmaceutically acceptable salt of 4-PBA is sodium phenylbutyrate.

55. The method of embodiment 54, wherein the sodium phenylbutyrate is administered at an amount of about 0.5 to about 10 grams per day.

56. The method of embodiment 54, wherein the sodium phenylbutyrate is administered at an amount of about 4.5 to about 8.5 grams per day.

57. The method of embodiment 54, wherein the sodium phenylbutyrate is administered at an amount of about 3 grams twice a day.

58. The method of any one of the proceeding embodiments, further comprising administering to the human subject one or more additional therapeutic agent.

59. The method of any one of the proceeding embodiments, wherein the human subject has previously been treated with one or more additional therapeutic agent.

60. The method of embodiment 58, wherein the therapeutic agent is tacrine, rivastigmine, galantamine, donepezil, or memantine.

61. The method of any one of the proceeding embodiments, further comprising administering to the human subject a plurality of food items comprising solid foods or liquid foods.

62. The method of any one of the proceeding embodiments, wherein the human subject is about 18 years or older.

What is claimed is:

1. A method of treating at least one symptom of progressive supranuclear palsy (PSP), the method comprising administering to the subject a pharmaceutically effective amount of a combination of TURSO and sodium phenylbutyrate.

2. A method of treating at least one symptom of Alzheimer's disease (AD) in a human subject, the method comprising administering to the human subject a pharmaceutically effective amount of a combination of TURSO and sodium phenylbutyrate, wherein the human subject:
(a) carries one or more copies of the APOEε4 allele;
(b) has a cerebral spinal fluid (CSF) level of total tau of about 300 μg/mL or higher; or
(c) has a CSF level of phospho-tau of about 70 μg/mL or higher.

3. The method of claim 2, wherein the method comprises, prior to administration, a step of determining whether the human subject has at least one of the characteristics of (a)-(c).

4. The method of claim 2, wherein the human subject has a CSF level of total tau of about 300 μg/mL or higher.

5. The method of claim 2, wherein the human subject has a CSF level of phospho-tau of about 70 μg/mL or higher.

6. A method of slowing Alzheimer's disease (AD) progression in a human subject having one or more symptoms of AD, the method comprising administering to the subject a pharmaceutically effective amount of a combination of TURSO and sodium phenylbutyrate.

7. A method of increasing survival time of a human subject having one or more symptoms of Alzheimer's disease, the method comprising administering to the subject a pharmaceutically effective amount of a combination of TURSO and sodium phenylbutyrate.

8. A method of decreasing the level of total CSF tau, decreasing the level of CSF phospho-tau, increasing CSF Aβ₁₋₄₂/Aβ₁₋₄₀, or increasing the level of CSF 8-OHDG in a human subject having one or more symptoms of Alzheimer's disease, the method comprising administering to the subject a pharmaceutically effective amount of a combination of TURSO and sodium phenylbutyrate.

9. The method of claim 8, wherein the phospho-tau species is phospho-tau 181.

10. A method of treating and/or preventing a tauopathy in a human subject, the method comprising administering to the human subject a pharmaceutically effective amount of a combination of TURSO and sodium phenylbutyrate.

11. The method of claim 10, wherein the subject has a baseline CSF total tau level of about 300 μg/mL or higher.

12. The method of claim 10, wherein the tauopathy is progressive supranuclear palsy (PSP), frontotemporal lobar degeneration (FTLD-TAU), corticobasal degeneration, Pick's disease, argyrophilic grain disease, post-encephalitic parkinsonism, chronic traumatic encephalopathy, primary age-related tauopathy, stroke, traumatic brain injury, or Alzheimer's disease.

13. The method of claim 10, wherein the tauopathy is progressive supranuclear palsy.

14. A method of treating and/or preventing an amyloidosis related condition in a human subject, the method comprising administering to the human subject a pharmaceutically effective amount of a combination of TURSO and sodium phenylbutyrate.

15. A method comprising administering to a human subject at risk for developing Alzheimer's disease a pharmaceutically effective amount of a combination of TURSO and sodium phenylbutyrate.

16. The method of claim 15, wherein the subject is determined to be at risk for developing Alzheimer's disease by evaluating a level of a biomarker in a biological sample obtained from the subject.

17. The method of claim 16, wherein the biomarker is total tau or phospho-tau.

18. The method of claim 16, wherein the biological sample is CSF.

19. The method of claim 15, wherein the subject carries one or more copies of the APOEε4 allele.

20. The method of claim 15, wherein the subject carries one or more mutations in at least one gene selected from the group consisting of: APP, PSEN1, and PSEN2.

21. A method of decreasing the CSF levels of FABP3, neurogranin, YKL-40, or IL-15 in a human subject having one or more symptoms of Alzheimer's disease, the method comprising administering to the subject a pharmaceutically effective amount of a combination of TURSO and sodium phenylbutyrate.

22. A method of treating at least one symptom of a neurodegenerative disease characterized by elevated total tau levels or phospho-tau levels, the method comprising administering to the subject a pharmaceutically effective amount of a combination of TURSO and sodium phenylbutyrate.

23. A method of treating at least one symptom of a neurodegenerative disease characterized by elevated YKL-40 levels, the method comprising administering to the subject a pharmaceutically effective amount of a combination of TURSO and sodium phenylbutyrate.

24. The method of any one of claims 22-23, wherein the neurodegenerative disease is Alzheimer's disease.

25. The method of any one of claims 22-23, wherein the neurodegenerative disease is PSP.

26. The method of any one of claims 22-23, wherein the neurodegenerative disease is cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration (FTLD-TAU), corticobasal degeneration, Pick's disease, argyrophilic grain disease, post-encephalitic parkinsonism, chronic traumatic encephalopathy, primary age-related tauopathy, stroke, traumatic brain injury, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, subacute sclerosing panencephalitis, Tangle only dementia, multi-infarct dementia, or ischemic stroke.

27. A method of decreasing the level of CSF YKL-40, decreasing the level of Ptpn1, or increasing the CSF ratio of 33 kDa tau to 55 kDa tau in a human subject having one or more symptoms of PSP, the method comprising administering to the subject a pharmaceutically effective amount of a combination of TURSO and sodium phenylbutyrate.

28. The method of any one of the above claims, wherein the TURSO and the sodium phenylbutyrate are administered once a day or twice a day.

29. The method of any one of the above claims, wherein TURSO is administered to the subject at a dose of about 5 mg/kg to about 100 mg/kg.

30. The method of any one of the above claims, wherein sodium phenylbutyrate is administered to the subject at a dose of about 10 mg/kg to about 400 mg/kg.

31. The method of any one of the above claims, wherein the TURSO is administered at an amount of about 0.5 to about 5 grams per day.

32. The method of any one of the above claims, wherein the sodium phenylbutyrate is administered at an amount of about 0.5 grams to about 10 grams per day.

33. The method of any one of the above claims, comprising administering to the subject 1 gram of TURSO and 3 grams of sodium phenylbutyrate once a day or twice a day.

34. The method of any one of the above claims, comprising administering to the subject 1 gram of TURSO once a day and 3 grams of sodium phenylbutyrate once a day for about 14 days or more, followed by administering to the subject about 1 gram of TURSO twice a day and 3 grams of sodium phenylbutyrate twice a day.

35. The method of any one of the above claims, wherein the TURSO and the sodium phenylbutyrate are administered orally.

36. The method of any one of the above claims, wherein the TURSO and the sodium phenylbutyrate are formulated as a single powder formulation.

37. The method of any one of the above claims, further comprising administering one or more additional therapeutic agents to the subject.

38. The method of claim 37, wherein the therapeutic agent is tacrine, rivastigmine, galantamine, donepezil, or memantine.

39. The method of any one of the proceeding claims, further comprising administering to the human subject a plurality of food items comprising solid foods or liquid foods.

40. The method of any one of the proceeding claims, wherein the human subject is about 18 years or older.
